SEQUENCE LISTING (27 sequences)

SEQ ID NO: 1 - protein, 11 aa, Artificial Sequence
  Synthetic Polypeptide
AANDENYALAA

SEQ ID NO: 2 - protein, 15 aa, Artificial Sequence
  Synthetic Polypeptide
AANDENYSENYADAS

SEQ ID NO: 3 - protein, 14 aa, Artificial Sequence
  Synthetic Polypeptide
AADENYSENYADAS

SEQ ID NO: 4 - DNA, 27 bp, Artificial Sequence
  Synthetic Polynucleotide
caattcattaaagaggagaaaggatcc

SEQ ID NO: 5 - DNA, 27 bp, Artificial Sequence
  Synthetic Polynucleotide
caattcattaaagagctgaaaggatca

SEQ ID NO: 6 - DNA, 27 bp, Artificial Sequence
  Synthetic Polynucleotide
caattcattaaagagtcgaaaggatca

SEQ ID NO: 7 - DNA, 27 bp, Artificial Sequence
  Synthetic Polynucleotide
caattcattaaagaggcgaaaggatcc

SEQ ID NO: 8 - DNA, 27 bp, Artificial Sequence
  Synthetic Polynucleotide
caattcattaaagagttgaaaggatcc

SEQ ID NO: 9 - DNA, 27 bp, Artificial Sequence
  Synthetic Polynucleotide
caattcattaaagagtggaaaggatcc

SEQ ID NO: 10 - DNA, 54 bp, Artificial Sequence
  Synthetic Polynucleotide
tccctatcagtgatagagattgacatccctatcagtgatagagatactgagcac

SEQ ID NO: 11 - DNA, 40 bp, Artificial Sequence
  Synthetic Polynucleotide
ctgtcataaagttgtcacggccgagacttatagtcgcttt

SEQ ID NO: 12 - DNA, 60 bp, Artificial Sequence
  Synthetic Polynucleotide
ccataagattagcggatcctacctgacgctttttatcgcaactctctactgtttctccat

SEQ ID NO: 13 - DNA, 274 bp, Artificial Sequence
  Synthetic Polynucleotide
gcagattgagtaaccgtgaatgtttgtacaaatgtttcaaagatgttactatgagtgtcccggccagcatcactttatattttgtgacgtctggccggacgttttccctagtgttggctgttttagcgacctggccgtacaggtcaggtttttttttaccgctaaacaactgaagccattgtaacctctgaatgattcattgtaagttactcttaagtatcatcttgcctgtactatagtgcaggttaagtccacgttaagtaaaagaagcagc

SEQ ID NO: 14 - DNA, 37 bp, Artificial Sequence
  Synthetic Polynucleotide
tcgacatcaggaaaatttttctgatacttacagccat

SEQ ID NO: 15 - DNA, 38 bp, Artificial Sequence
  Synthetic Polynucleotide
tcgacataaagtctaacctataggatacttacagccat

SEQ ID NO: 16 - DNA, 184 bp, Artificial Sequence
  Synthetic Polynucleotide
gctcacaacagtgtaagcgtatccgttattgtttgattttcaaggaaaaaagaaaacattcaggctccatgctgcttcttttacttaacgtggacttaacctgcactatagtacaggcaagatgatacttaagagtaacttacaatgaatcattcagaggttacaatggcttcagttgtttagc

SEQ ID NO: 17 - DNA, 36 bp, Artificial Sequence
  Synthetic Polynucleotide
ttcacttttaatcatccggctcgtataatgtgtgga

SEQ ID NO: 18 - DNA, 35 bp, Artificial Sequence
  Synthetic Polynucleotide
ttgcctcttaatcatcggctcgtataatgtgtgga

SEQ ID NO: 19 - DNA, 36 bp, Artificial Sequence
  Synthetic Polynucleotide
ttgacatcaggaaaatttttctgtataatgtgtgga

SEQ ID NO: 20 - DNA, 58 bp, Artificial Sequence
  Synthetic Polynucleotide
ctaggtttctccatacccgtttttttgggctagcgaattcaaagaggagaaatactag

SEQ ID NO: 21 - DNA, 26 bp, Artificial Sequence
  Synthetic Polynucleotide
caattcattaaagaggagaaggatcc

SEQ ID NO: 22 - DNA, 44 bp, Artificial Sequence
  Synthetic Polynucleotide
acccgtttttttgggctagcgaattcaaagaggagaaatactag

SEQ ID NO: 23 - DNA, 85 bp, Artificial Sequence
  Synthetic Polynucleotide
gggcccaagttcacttaaaaaggagatcaacaatgaaagcaattttcgtactgaaacatcttaatcatgcgagggatggtttcta

SEQ ID NO: 24 - DNA, 85 bp, Artificial Sequence
  Synthetic Polynucleotide
gggcccaagttcacttaaaaaggagatcaacaatgaaagcaattttcgtactgaaacatcttaatcatgcctaggaagttttcta

SEQ ID NO: 25 - DNA, 45 bp, Artificial Sequence
  Synthetic Polynucleotide
gcggcgaacgatgaaaactatagcgaaaactatgcggatgcgagc

SEQ ID NO: 26 - DNA, 33 bp, Artificial Sequence
  Synthetic Polynucleotide
gctgctaacgacgaaaactacgctctggctgct

SEQ ID NO: 27 - DNA, 162 bp, Artificial Sequence
  Synthetic Polynucleotide
tccctatcagtgatagagattgacatccctatcagtgatagagatactgagcacctaggtttctccatacccgtttttttgggctagcgaattcaaagaggagaaatactagatggatttgtcacagctaacaccacgtcgtccctatctgctgcgtgcatt

(19) United States
(12) Patent Application Publication
(10) Pub. No.: US 2017/0130210 A1
(43) Pub. Date: May 11, 2017

(54) DYNAMIC KNOCKDOWN OF CENTRAL METABOLISM FOR REDIRECTING GLUCOSE-6-PHOSPHATE FLUXES

(71) Applicant: Massachusetts Institute of Technology, Cambridge, MA (US)

(72) Inventors: Irene Marie BROCKMAN, Cambridge, MA (US); Kristala Lanett Jones PRATHER, Milton, MA (US); Apoorv GUPTA, Cambridge, MA (US)

(73) Assignee: MASSACHUSETTS INSTITUTE OF TECHNOLOGY, Cambridge, MA (US)

(21) Appl. No.: 15/317,704

(22) PCT Filed: Jun. 9, 2015
(86) PCT No.: PCT/US2015/034973

§ 371 Date: Dec. 9, 2016

Related U.S. Application Data
(60) Provisional application No. 62/009,672, filed on Jun. 9, 2014.

(51) Int. Cl. C12N 9/12; C12P 7/22; C12P 19/02

(52) U.S. Cl. CPC C12N 9/1205; C12P 19/02; C12Y 207/01011; C12P 7/22

(57) ABSTRACT

Described herein are methods for dynamic redirection of metabolic flux in a cell from central metabolism towards production of heterologous products.

### RELATED APPLICATIONS

This application claims the benefit under 35 U.S.C §119(e) of U.S. Provisional Application Ser. No. 62/009,672, entitled “DYNAMIC KNOCKDOWN OF CENTRAL METABOLISM FOR REDIRECTING GLUCOSE-6-PHOSPHATE FLUXES,” filed on Jun. 9, 2014, which is herein incorporated by reference in its entirety.

### FEDERALLY SPONSORED RESEARCH

This invention was made with Government support under Grant No. GM008334, awarded by the National Institutes of Health and Grant No. CBET0954986, awarded by the National Science Foundation. The Government has certain rights in this invention.

### BACKGROUND OF INVENTION

Control of native metabolic enzyme levels is an important part of engineering strains for overproduction of heterologous compounds, such as biofuels, biopolymers, and molecules with therapeutic properties. However, for many enzymes involved in central metabolism, static knockdown may lead to undesired consequences, such as poor growth of the engineered strain and/or poor expression of recombinant proteins, all of which can result in low product yield.

### SUMMARY OF INVENTION

Described herein are methods for metabolic engineering in which glucose-6-phosphate metabolite flux is redirected to increase production of heterologous compounds, such as myo-inositol, glucuronic acid, and/or glucaric acid. Aspects of the disclosure relate to methods and cells for redirecting the flux of glycolytic intermediates (e.g., glucose-6-phosphate) in a cell and methods of producing recombinant cells for the production of heterologous compounds (e.g., myo-inositol, glucuronic acid, and/or glucaric acid).

In some aspects, methods of redirecting flux of glucose-6-phosphate in a recombinant cell are provided. In some embodiments, the method comprises regulating activity of a phosphofructokinase-1 (pfk-1). In some embodiments, the method further comprises expressing in the cell a heterologous pathway that can utilize a glycolytic intermediate. In some embodiments, the glycolytic intermediate is glucose-6-phosphate. In some embodiments, the heterologous pathway comprises expressing a myo-inositol-1-phosphate synthase.

In some embodiments, the method further comprises reducing expression of a glucose-6-phosphate dehydrogenase (zwf). In some embodiments, the cell does not express glucose-6-phosphate dehydrogenase.

In some embodiments, regulating activity of the phosphofructokinase-1 protein comprises reducing the amount of phosphofructokinase-1 protein in the cell. In some embodiments, the amount of phosphofructokinase-1 protein is reduced by at least 50%. In some embodiments, the amount of phosphofructokinase-1 protein is reduced by at least 75%. In some embodiments, the amount of phosphofructokinase-1 protein is reduced by at least 90%.

In some embodiments, reducing the amount of phosphofructokinase-1 protein comprises degrading the phosphofructokinase-1 protein. In some embodiments, reducing the amount of phosphofructokinase-1 protein comprises targeting the phosphofructokinase-1 protein for degradation by a protease. In some embodiments, the phosphofructokinase-1 protein is fused to a peptide tag. In some embodiments, the peptide tag is an SsrA tag. In some embodiments, the method further comprises expressing in the cell an adaptor protein. In some embodiments, the adaptor protein is SspB, which targets the phosphofructokinase-1 protein for degradation.

In some embodiments, SspB is expressed under the control of a first inducible promoter. In some embodiments, the method further comprises contacting the cell with a first inducer. In some embodiments, the myo-inositol-1-phosphate synthase (INO1) is expressed under the control of an inducible promoter. In some embodiments, the method further comprises contacting the cell with a second inducer. In some embodiments, the first inducer is anhydrotetracycline (aTc). In some embodiments, the second inducer is isopropyl-β-D-1-thiogalactopyranoside.

In some embodiments, the gene encoding the myo-inositol-1-phosphate synthase is a Saccharomyces gene. In some embodiments, the cell is a microbial cell. In some embodiments, the microbial cell is a bacterial cell. In some embodiments, the bacterial cell is an Escherichia coli cell. In some embodiments, the cell is a eukaryotic cell. In some embodiments, the eukaryotic cell is a fungal cell, a yeast cell, an insect cell, a plant cell, or a mammalian cell.

In some embodiments, the method provided is a method of producing myo-inositol, wherein the method further comprises culturing the cell and optionally recovering myo-inositol from the cell and/or cell culture.

In some embodiments, the method further comprises expressing in the cell a gene encoding a myo-inositol oxygenase. In some embodiments, the method further comprises expressing in the cell a gene encoding a uronate dehydrogenase. In some embodiments, the method provided is a method of producing glucuronic acid, and the method further comprises culturing the cell and optionally recovering glucuronic acid from the cell and/or cell culture. In some embodiments, the method provided is a method of producing glucaric acid, and the method further comprises culturing the cell and optionally recovering glucaric acid from the cell and/or cell culture.

In some embodiments, the method further comprises reducing expression of a glucarate dehydratase protein. In some embodiments, the method further comprises mutating a gudD gene in the recombinant cell. In some embodiments, the method further comprises reducing the expression of a uronate isomerase protein. In some embodiments, the method further comprises mutating a uxaC gene in the recombinant.

In some embodiments, the method further comprises reducing expression of the phosphofructokinase-II protein. In some embodiments, reducing expression of the phosphofructokinase-II protein comprises eliminating expression the phosphofructokinase-II protein. In some embodiments, a gene encoding the adaptor protein is integrated into the genome of the cell. In some embodiments, the gene encoding the adaptor protein is integrated at a phage attachment site. In some embodiments, the phage attachment site is HK022.

In other aspects, a method for producing a recombinant cell is provided, comprising expressing in the cell a regulatable phosphofructokinase protein (Pfk-1) and a means of regulating the phosphofructokinase protein. In some embodiments, the method further comprises expressing in the cell a heterologous pathway that can utilize a glycolytic intermediate. In some embodiments, the glycolytic intermediate is glucose-6-phosphate. In some embodiments, the recombinant cell has increased production of myo-inositol.

In some embodiments, the method further comprises expressing in the cell a myo-inositol-1-phosphate synthase (INO1). In some embodiments, the method further comprises reducing expression of a glucose-6-phosphate dehydrogenase gene (zwf).

In some embodiments, the method further comprises reducing expression of a glucarate dehydratase protein. In some embodiments, the method further comprises mutating a gudD gene in the recombinant cell. In some embodiments, the method further comprises reducing the expression of a uronate isomerase protein. In some embodiments, the method further comprises mutating a uxaC gene in the recombinant.

In some embodiments, the cell is cultured in the presence of glucose. In some embodiments, the cell is cultured in the presence of arabinose. In some embodiments, the cell is cultured in the presence of xylose.

In some embodiments, the cell is a microbial cell. In some embodiments, the microbial cell is a bacterial cell. In some embodiments, the bacterial cell is an Escherichia coli cell. In some embodiments, the cell is a eukaryotic cell. In some embodiments, the eukaryotic cell is a fungal cell, a yeast cell, an insect cell, a plant cell, or a mammalian cell.

In other aspects, recombinant cells are provided that express a regulatable phosphofructokinase protein (Pfk-1) and a means of regulating the phosphofructokinase protein. In some embodiments, the cell further expresses a heterologous pathway that can utilize a glycolytic intermediate. In some embodiments, the glycolytic intermediate is glucose-6-phosphate.

In some embodiments, the cell further expresses a myo-inositol-1-phosphate synthase (INO1). In some embodiments, the cell has reduced expression of a glucose-6-phosphate dehydrogenase gene (zwf). In some embodiments, the cell does not express glucose-6-phosphate dehydrogenase.

In some embodiments, the amount of the phosphofructokinase-1 protein is reduced in the cell. In some embodiments, the amount of phosphofructokinase-1 protein is reduced by at least 50%. In some embodiments, the amount of phosphofructokinase-1 protein is reduced by at least 75%. In some embodiments, the amount of phosphofructokinase-1 protein is reduced by at least 90%.

In some embodiments, the amount of phosphofructokinase-1 protein is reduced by degrading the phosphofructokinase-1 protein. In some embodiments, the amount of phosphofructokinase-1 protein is reduced by targeting the phosphofructokinase-1 protein for degradation by a protease. In some embodiments, the phosphofructokinase-1 protein is fused to a peptide tag. In some embodiments, the peptide tag is an SsrA tag. In some embodiments, the cell expresses an adaptor protein. In some embodiments, the adaptor protein is SspB, which targets the phosphofructokinase-1 protein for degradation.

In some embodiments, SspB is expressed under the control of a first inducible promoter. In some embodiments, the cell is contacted with a first inducer. In some embodiments, the first inducer is anhydrotetracycline (aTc). In some embodiments, the myo-inositol-1-phosphate synthase (INO1) is expressed under the control of an inducible promoter. In some embodiments, the cell is contacted with a second inducer. In some embodiments, the second inducer is isopropyl-β-D-1-thiogalactopyranoside (IPTG).

In some embodiments, the gene encoding the myo-inositol-1-phosphate synthase is a Saccharomyces gene. In some embodiments, the cell is a microbial cell. In some embodiments, the microbial cell is a bacterial cell. In some embodiments, the bacterial cell is an Escherichia coli cell. In some embodiments, the cell is a eukaryotic cell. In some embodiments, the eukaryotic cell is a fungal cell, a yeast cell, an insect cell, a plant cell, or a mammalian cell.

In some embodiments, the cell expresses a gene encoding a myo-inositol oxygenase. In some embodiments, the cell expresses a gene encoding a uronate dehydrogenase. In some embodiments, the cell has reduced expression of the phosphofructokinase-II protein. In some embodiments, the cell does not express phosphofructokinase-II.

In some embodiments, the recombinant cell has reduced expression of glucarate dehydratase. In some embodiments, the recombinant cell does not express glucarate dehydratase. In some embodiments, an endogenous gudD gene of the recombinant cell is mutated. In some embodiments, the recombinant cell has reduced expression of uronate isomerase. In some embodiments, the recombinant cell does not express uronate isomerase. In some embodiments, an endogenous uxaC gene of the cell is mutated.

In some embodiments, a gene encoding the adaptor protein is integrated into the genome of the cell. In some embodiments, the gene encoding the adaptor protein is integrated at a phage attachment site. In some embodiments, the phage attachment site is HK022.

In other aspects, a method of producing myo-inositol is provided. In some embodiments, the method comprises culturing any of the cells described herein produce myo-inositol. In some embodiments, the method comprises recovering the myo-inositol from the cell culture. In some embodiments, the cell is cultured in the presence of glucose. In some embodiments, the cell is cultured in the presence of arabinose. In some embodiments, the cell is cultured in the presence of xylose.

In some embodiments, the method further comprises recovering the glucuronic acid or glucaric acid from the cell culture. In some embodiments, the cell is cultured in the presence of glucose. In some embodiments, the cell is cultured in the presence of arabinose. In some embodiments, the cell is cultured in the presence of xylose.

In other aspects, a cell culture is produced by culturing any of the cells provided herein. In some embodiments, the cell culture contains at least 100 mg L⁻¹ myo-inositol. In some embodiments, the cell culture contains at least 500 mg L⁻¹ myo-inositol. In some embodiments, the cell culture contains at least 100 mg L⁻¹ glucuronic acid. In some embodiments, the cell culture contains at least 500 mg L⁻¹ glucuronic acid. In some embodiments, the cell culture contains at least 100 mg L⁻¹ glucaric acid. In some embodiments, the cell culture contains at least 500 mg L⁻¹ glucaric acid.

In other aspects, a supernatant of a cell culture is provided. In some embodiments, the supernatant is produced by culturing any of the cells provided herein. In some embodiments, the supernatant contains at least 100 mg L⁻¹ myo-inositol. In some embodiments, the supernatant contains at least 500 mg L⁻¹ myo-inositol. In some embodiments, the supernatant contains at least 100 mg L⁻¹ glucuronic acid. In some embodiments, the supernatant contains at least 500 mg L⁻¹ glucuronic acid. In some embodiments, the supernatant contains at least 100 mg L⁻¹ glucaric acid. In some embodiments, the supernatant contains at least 500 mg L⁻¹ glucaric acid.

Aspects of the invention relate to methods of autonomously redirecting flux of glucose-6-phosphate in a recombinant cell, the method comprising regulating a phosphofructokinase-1 (pfk-1) in the recombinant cell, wherein the pfk-1 is regulated based on quorum sensing or nutrient sensing. In some embodiments, regulating activity of the phosphofructokinase-1 protein comprises reducing the amount of phosphofructokinase-1 protein in the cell. In some embodiments, the amount of phosphofructokinase-1 protein is reduced by at least 50%, at least 75% or at least 90%.

In some embodiments, reducing the amount of phosphofructokinase-1 protein comprises degrading the phosphofructokinase-1 protein. In some embodiments, reducing the amount of phosphofructokinase-1 protein comprises targeting the phosphofructokinase-1 protein for degradation by a protease.

In some embodiments, the phosphofructokinase-1 protein is fused to a peptide tag. In some embodiments, the peptide tag is an SsrA tag. In some embodiments, methods further comprising expressing in the cell an adaptor protein. In some embodiments, the adaptor protein is SspB and targets the phosphofructokinase-1 protein for degradation.

In some embodiments, SspB is expressed under the control of a first inducible promoter. In some embodiments, the first inducible promoter is responsive to a molecule produced by the recombinant cell. In some embodiments, the first inducible promoter is responsive to a quorum sensing molecule. In some embodiments, the first inducible promoter is a PesaS promoter or a PeasR promoter. In some embodiments, the first inducible promoter is from Pantoea stewartii. In some embodiments, the quorum sensing molecule is 3-oxohexanoyl-homoserine-lactone (30C6HSL).

In some embodiments, methods further comprise expressing in the cell a gene encoding a quorum sensing transcription factor. In some embodiments, the quorum sensing transcription factor is EsaR. In some embodiments, the quorum sensing transcription factor is from Pantoea stewartii. In some embodiments, methods further comprise expressing in the cell a gene encoding a quorum sensing molecule synthase. In some embodiments, the quorum sensing molecule synthase is a 3OC6HSL synthase. In some embodiments, the quorum sensing molecule synthase is EsaI. In some embodiments, the quorum sensing molecule synthase is from Pantoea stewartii.

In some embodiments, the activity of pfk-1 is regulated based on the level of a nutrient. In some embodiments, the nutrient is phosphate, arabanose, glucose or tryptophan. In some embodiments, the first inducible promoter is responsive to a level of phosphate. In some embodiments, the first inducible promoter is a phoA promoter. In some embodiments, the first inducible promoter is a phoA promoter variant. In some embodiments, the phoA promoter variant is apFAB114 or apFAB104.

In some embodiments, methods further comprise contacting the cell with phosphate. In some embodiments, the nutrient is arabanose. In some embodiments, the first inducible promoter is responsive to a level of arabanose. In some embodiments, the first inducible promoter is a PBAD promoter. In some embodiments, methods further comprise contacting the cell with glucose. In some embodiments, methods further comprising contacting the cell with arabinose. In some embodiments, methods further comprise contacting the cell with xylose.

In some embodiments, methods further comprise expressing in the cell a heterologous pathway that can utilize a glycolytic intermediate. In some embodiments, the glycolytic intermediate is glucose-6-phosphate. In some embodiments, wherein the heterologous pathway comprises expressing a myo-inositol-1-phosphate synthase. In some embodiments, methods further comprise reducing expression of a glucose-6-phosphate dehydrogenase (zwf).

In some embodiments, the cell does not express glucose-6-phosphate dehydrogenase. In some embodiments, the myo-inositol-1-phosphate synthase (INO1) is expressed under the control of an inducible promoter. In some embodiments, methods further comprise contacting the cell with a second inducer. In some embodiments, the second inducer is isopropyl-β-D-1-thiogalactopyranoside.

In some embodiments, the gene encoding the myo-inositol-1-phosphate synthase is a Saccharomyces gene. In some embodiments, the cell is a microbial cell. In some embodiments, the microbial cell is a bacterial cell. In some embodiments, the bacterial cell is an Escherichia coli cell. In some embodiments, the cell is a eukaryotic cell. In some embodiments, the eukaryotic cell is a fungal cell, a yeast cell, an insect cell, a plant cell, or a mammalian cell.

In some embodiments, the method is a method of producing myo-inositol, wherein the method further comprises culturing the cell and optionally recovering myo-inositol from the cell and/or cell culture. In some embodiments, methods further comprise expressing in the cell a gene encoding a myo-inositol oxygenase. In some embodiments, methods further comprise expressing in the cell a gene encoding a uronate dehydrogenase.

In some embodiments, the method is a method of producing glucuronic acid, and the method further comprises culturing the cell and optionally recovering glucuronic acid from the cell and/or cell culture. In some embodiments, the method is a method of producing glucaric acid, and the method further comprises culturing the cell and optionally recovering glucaric acid from the cell and/or cell culture.

In some embodiments, the method further comprises reducing expression of a glucarate dehydratase protein. In some embodiments, the method further comprises mutating a gudD gene in the recombinant cell. In some embodiments, the method further comprises reducing the expression of a uronate isomerase protein. In some embodiments, the method further comprises mutating a uxaC gene in the recombinant cell.

In some embodiments, methods further comprise reducing expression of the phosphofructokinase-II protein. In some embodiments, reducing expression of the phosphofructokinase-II protein comprises eliminating expression the phosphofructokinase-II protein. In some embodiments, a gene encoding the adaptor protein is integrated into the genome of the cell. In some embodiments, the gene encoding the adaptor protein is integrated at a phage attachment site. In some embodiments, the phage attachment site is HK022.

Further aspects of the invention relate to methods for producing a recombinant cell, comprising expressing in the cell an autonomously regulatable phosphofructokinase protein (Pfk-1) and a means of autonomously regulating the Pfk-1 protein, wherein the Pfk-1 protein is regulated based on quorum sensing or nutrient sensing.

Further aspects of the invention relate to a recombinant cell, that expresses a regulatable phosphofructokinase protein (Pfk-1) and a means of autonomously regulating the Pfk-1 protein, wherein the Pfk-1 protein is regulated based on quorum sensing or nutrient sensing.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. The figures are illustrative only and are not required for enablement of the disclosure. For purposes of clarity, not every component may be labeled in every drawing. In the drawings:

FIG. 1 shows kinetic analysis of flux through a myo-inositol-1-phosphate synthase, INO1. FIG. 1A shows predicted changes in flux through INO1 as the levels of the native metabolic enzymes Pgi, Pfk, and Zwf are varied. FIG. 1B shows sensitivity analysis indicating how predicted flux improvements through Pfk knockdown vary with INO1 K_{M}.

FIG. 2 shows the construction of a switchable strain for myo-inositol and glucaric acid production. FIG. 2A presents a schematic of glucose-6-phosphate in the cell. Gene knockouts, indicated with an “X,” were made so that Pfk-1 was the sole control point for glucose-6-phosphate utilization. FIG. 2B shows modifications made to the pfkA and HK022 loci to generate aTc-inducible control of Pfk-I degradation.

FIG. 3 shows performance of E. coli strain IB1863 in growth (Pfk-I ON) and production modes (Pfk-I OFF). FIG. 3A shows the baseline growth of E. coli strain IB1863 in comparison to the parent E. coli strain, IB531. FIG. 3B shows a decline in Pfk activity in response to induction of SspB with aTc to allow rapid degradation of Pfk-I protein. FIG. 3C shows a Western blot confirming disappearance of Pfk-I protein from crude lysates. Lane 1: Western C ladder, lane 2: Initial culture of IB1863 before split for aTc treatment (t=0), lanes 3-5: Untreated IB1863 (t=1, 4, 16 hours), lane 6-8: IB1863 treated with aTc (t=1, 4, 16, hours after treatment). FIG. 3D depicts the growth rate of E. coli strain IB1863 as a function of aTc concentration added to the medium at inoculation. The dotted line represents fit to Hill function with n=5. All points represent triplicate mean±standard deviation.

FIG. 4 shows the effect of Pfk-I knockdown on glucose consumption and acetate production. FIG. 4A shows glucose consumption and acetate production rates in IB1863 and IB 531. Glucose consumption was measured at 4 hours and 16 hours after addition of aTc to the indicated cultures, and uptake rate was averaged across the given time period. Plot depicts the triplicate mean±standard deviation. FIG. 4B shows intracellular G6P and F6P pools in IB1863 and IB531. Cells were collected in exponential phase and intracellular metabolites were extracted into boiling 75% ethanol. For cultures treated with aTc, cells were collected one hour after aTc addition to the culture. Plot depicts the triplicate mean±SD.

FIG. 5 shows the effect of Pfk-I knockdown on myo-inositol production from glucose. FIG. 5A shows growth of IB531-I and IB1863-I with aTc added at times varying from 0 to 47 hours. Points represent duplicate mean±SD. FIG. 5B shows Pfk activity in all cultures at 48 hours as a function of SspB induction time. “No SspB” refers to IB1863-I without induction of SspB by aTc addition and IB531-I shows the MG1655 ΔendA control. FIG. 5C shows yield and titer of MI at 78 hours as a function of SspB induction time in IB1863-I. FIG. 5D depicts a SDS-PAGE gel showing representative INO1 expression (indicated at arrow) in IB531-I and IB1863-I. Lane 1: ladder, lanes 2-5: expression at 18 hours (IB531-I, IB1863-I with aTc at t=11.5 hours, IB1863-I with aTc at t=18 hours, IB18634 without aTc), lanes 6-10: expression at 48 hours (IB531-I, IB1863-I with aTc at t=11.5 hours, IB1863-I with aTc at t=18 hours, IB1863-I with aTc at t=32 hours, IB1863-I without aTc). Plots depict duplicate mean±SD for cultures with timed SspB induction and triplicate mean±SD for uninduced IB18634 and IB531-I controls.

FIG. 6 shows the characterization of IB1014 in modified MOPS minimal medium+10 g/L glucose at 30° C. FIG. 6A shows growth of IB1014 without aTc addition and with aTc addition at OD=0.06. FIG. 6 B shows intracellular G6P and F6P pools in IB1014 with and without induction of SspB by aTc addition. Cells were collected in exponential phase and intracellular metabolites were extracted into boiling 75% ethanol. For cultures treated with aTc, cells were collected one hour after aTc addition to the culture. Plots depict the triplicate mean±SD.

FIG. 7 shows yields (white bars) and titers (gray bars) of glucaric acid produced by IB1486-GA as a function of aTc addition time. Glucaric acid production was measured at 48 hours in T12 medium supplemented with 15 g/L glucose. Error bars represent triplicate mean±SD.

FIG. 8 shows growth profiles and Pfk activity at 48 hours for IB1486-GA in T12+15 g/L glucose. FIG. 8A shows growth of IB1486-GA with aTc addition at the times indicated. Error bars represent triplicate mean±SD. FIG. 8B shows Pfk activity measured in selected wells from screening plate at 48 hours.

FIG. 9 shows the locations of IS2 insertions in the sspB expression cassette (SEQ ID NO: 27). Three instances of the IS2 insertion were found during sequencing. One insertion was immediately in front of the sspB coding sequence (insertion site 1), while the remaining two were at the same site, 36 bp into the sspB coding sequence (insertion sites 2 and 3).

FIG. 10 shows glucose release via starch hydrolysis in T12 medium. Amyloglucosidase additions are indicated by arrows: 0.006 U/ml at 12 and 36 hours, 0.012 U/ml at 48 hours.

FIG. 11 shows growth and glucaric acid production in IB1486-GA in T12+3 g/L glucose+12 g/L starch. FIG. 11A shows titers and yields of glucaric acid at 72 hours for aTc addition times from 12-48 hours. FIG. 11B shows growth of IB1486-GA in T12+3 g/L glucose+12 g/L starch. Starch addition resulted in higher opacity of medium at start of fermentation, and changes in OD600 after amylase addition represent both cell growth and changes in opacity as starch was broken down. Error bars represent triplicate mean±SD.

FIG. 12 shows titers and yields of glucaric acid at 72 hours for IB1486-GA in T12+5 g/L glucose+10 g/L starch. Amyloglucosidase additions were carried out at 24 and 48 hours. Error bars represent triplicate mean±SD.

FIG. 13 shows yields and titers of glucaric acid in IB1486-GA and LB1458-GA in shake flasks with T12+5 g/L glucose+10 g/L starch. Amyloglucosidase additions were carried out at 18, 40, and 48 hours. Error bars represent triplicate mean±SD.

FIG. 14 shows growth and Pfk activity in IB1486-GA and LG1458-GA in T12+5 g/L glucose+10 g/L starch with amyloglucosidase addition at 18, 40, and 48 hours.

FIG. 14A shows growth of LG1458-GA and IB1486-GA with and without aTc addition at 24 hours after inoculation. FIG. 14B shows Pfk activity in these strains at 48 hours after inoculation. Error bars represent triplicate mean±SD.

FIG. 15 shows acetate production and Pfk activity in IB1486-GA and LG1458-GA in T12+15 g/L glucose. FIG. 15A shows acetate production at 24 and 48 hours in IB1486-GA and LG1458-GA in T12+15 g/L glucose. Cultures were carried out in 250 ml baffled shake flasks with the fill volume noted and 250 rpm shaking at 30° C. and 80% relative humidity. FIG. 15B shows Pfk activity at 24 hours after inoculation in T12+15 g/L glucose with 30 ml fill volume in 250 ml flasks. Error bars represent triplicate mean±SD.

FIG. 16 shows a characterization of strains IB643 and IB1509 with phosphate-starvation controlled SspB expression. FIG. 16A shows growth of IB643 and IB1509 on modified MOPS minimal medium with 10 g/L glucose and excess phosphate at 30° C., compared to the original parent IB531 (MG1655 ΔendA) and IB1863, with aTc inducible control of SspB expression. FIG. 16B shows Pfk activity in these strains. All cultures were initially growth in modified MOPS minimal medium with excess phosphate. The “no phosphate” samples were taken after cultures were spun down and re-suspended in phosphate-free modified MOPS for 1 hour. Error bars represent triplicate mean±SD.

FIG. 17 shows growth and Pfk activity in IB531-I (open) and IB15094 (filled). FIG. 17A shows growth curves for strains in modified MOPS minimal medium with 0.1 (square), 0.2 (circle), or 1 (diamond) g/L K₂HPO₄ and 10 g/L glucose in the medium. FIG. 17B shows Pfk activity of both strains in each of the culture conditions, measured at 24 and 48 hours. Error bars represent triplicate mean±SD.

FIG. 18 shows titers and yields of MI in IB531-I and IB1509-I. FIG. 18A shows titers of MI in IB5314 and IB15094 at 72 hours in modified MOPS minimal medium containing the specified amount of phosphate. FIG. 18B shows yields of MI in IB5314 and IB15094 at 72 hours in modified MOPS minimal medium containing the specified amount of phosphate. Error bars represent triplicate mean±SD.

FIG. 19 shows titers and yields of MI in IB1863-I and IB1509-I. FIG. 19A shows titers of MI in IB1863-I and IB1509-I at 72 hours in modified MOPS minimal medium containing the specified amount of phosphate. FIG. 19B shows yields of MI in IB1863-I and IB15094 at 72 hours in modified MOPS minimal medium containing the specified amount of phosphate. Error bars represent triplicate mean±SD.

FIG. 20 shows activity and growth of strains IB1448 and IB1449 with arabinose-inducible SspB. FIG. 20A shows Pfk activity in IB531, IB1448, and IB1449 in modified MOPS minimal medium with 10 g/L glucose at 30° C. Measurements with 0.2% arabinose addition were taken one hour after addition of arabinose to the medium, in the presence of excess glucose. FIG. 20B shows growth of IB1448 and IB1449 at 30° C. in modified MOPS minimal medium with 10 g/L glucose and addition of 0.2% arabinose at OD=0.4 for the cultures indicated. Error bars represent triplicate mean±SD.

FIG. 21 shows growth and activity of strain AG2350, with AHL-inducible SspB expression. FIG. 21A shows growth of AG2350 and the original parent strain IB531 in modified MOPS minimal medium with 10 g/L glucose at 30° C., and AHL addition at OD=0.6 for indicated cultures. FIG. 21B shows Pfk activity in AG2350 in the same medium. For the “with AHL” case, activity was sampled one hour after AHL addition. Error bars represent triplicate mean±SD.

FIG. 22 shows tests of growth and Pfk activity in strain IB646. FIG. 22A shows growth of IB646 in modified MOPS+10 g/L glucose with and without AHL addition at the time indicated by the arrow. Final OD₆₀₀ for IB646 remains at approximately 6, while the wild-type strain IB531 reaches final OD₆₀₀ values of 9-10.

FIG. 22B shows Pfk activity of IB646 with and without AHL addition in LB. IB1897, the parent strain without integrated EsaR is also shown, indicating that EsaR expression is required for activation of Pfk expression. Expected Pfk activity with wild-type pfkA promoter in this medium is approximately 0.8 U/mg total protein.

FIG. 23 shows growth and activity of strains derived from IB646 RBS library. FIG. 23A shows Pfk activity in IB646, IB531, and RBS library strains IB2351-2353 in M9+0.4% glycerol. Strains IB2351 and IB2352 with activity lower than that of IB531 did not show growth on glucose minimal medium. Error bars represent duplicate mean±SD. FIG. 23B shows representative growth curves of IB646 and IB2353 in modified MOPS minimal medium+1% glucose at 30 C. Final OD₆₀₀ values measured by spectrophotometer were 6.58±0.09 (IB646) and 7.98±0.11 (IB2353).

FIG. 24 shows Pfk activity in IB646 and IB2275 with plasmid-based EsaI expression. FIG. 24A shows IB646 with EsaI expression induced from pMMB-B0034-EsaI and pKVS-B0034-EsaI (filled symbols) or leaky expression (open symbols). FIG. 24B shows IB2275 with EsaI expression induced from pMMB-B0034-EsaI and pKVS-B0034-EsaI (filled symbols) or leaky expression (open symbols).

FIG. 25 shows growth and activity in IB646 and IB2275 with integrated EsaI expression cassettes. FIG. 25A shows growth of IB646 and IB1379 (native pfkA promoter) in comparison with IB646 variants containing various integrated EsaI expression cassettes. FIG. 25B shows biomass density at 36 hours as a function of predicted strength of promoter/RBS combination driving expression in IB646 background. FIG. 25C shows a time course of Pfk activity measured in IB646+L18 and IB646+L19 cultures in LB. Activity at t=0 is activity in stationary phase starter cultures. FIG. 25D shows a comparison of growth in IB646 and IB2275 with L18 and L19 EsaI expression cassettes.

FIG. 26 shows growth and MI production in strains with autonomous pfkA switching. FIG. 26A shows MI titers at 72 hours in modified MOPS minimal medium+10 g/L glucose for strains based on IB646 and IB2275. IB1379 contains the native pfkA promoter. FIG. 26B shows MI titers at glucose exhaustion (72-114 hours) in modified MOPS minimal medium+10 g/L glucose+0.2% casamino acids. Strains based on AG2349 were tested, along with the top performing strain from the previous test, IB2275+L19, and the control strain IB1379. FIG. 26C shows growth profiles in modified MOPS minimal medium+10 g/L glucose for the strains with titers given in FIG. 26A. FIG. 26D shows growth profiles in modified MOPS minimal medium+10 g/L glucose+0.2% casamino acids for the strains with titers given in FIG. 26B. Error bars represent duplicate mean±SD.

FIG. 27: FIG. 27A shows PfkA activities at 14 hrs and 24 hrs for strains containing different EsaI expression levels compared with the “No EsaI” control and wildtype control in MOPS medium. For each strain, 14 hrs is the left bar and 24 hrs is the right bar on the graph. FIG. 27B shows endpoint MI titer in MOPS medium for various strains and their corresponding PfkA activity at 14 hrs shows an optimum EsaI expression. FIG. 27C shows PfkA activities for a subset of strains tested in MOPS+0.2% casamino acids. For each strain, 14 hrs is the left bar and 24 hrs is the right bar on the graph. FIG. 27D shows MI titer improvements in QS-based knockdown of PfkA activity compared to wildtype control.

FIG. 28 shows MI and acetate levels at the end of fermentation in T12 media show advantages of downregulating Pfk expression fast enough in reducing unwanted acetate production due to excessive glycolytic flux.

FIG. 29 depicts degradation of Pfk-I (untagged). The identities of the lanes are as follows: (1) ladder (2) purified Pfk-I variant (3)-(10) reaction samples at 0, 2, 5, 10, 20, 30, 45, and 60 minutes, respectively. The Pfk-I band is noted with an arrow. The other bands are ClpX, ClpP, and creatine kinase.

FIG. 30 depicts degradation of Pfk-I (LAA). The identities of the lanes are as follows: (1) ladder (2) purified Pfk-I variant (3)-(10) reaction samples at 0, 2, 5, 10, 20, 30, 45, and 60 minutes, respectively. The Pfk-I band is noted with an arrow. The other bands are ClpX, ClpP, and creatine kinase.

FIG. 31 depicts degradation of Pfk-I (DAS+4). The identities of the lanes are as follows: (1) ladder (2) purified Pfk-I variant (3)-(10) reaction samples at 0, 2, 5, 10, 20, 30, 45, and 60 minutes, respectively. The Pfk-I band is noted with an arrow. The other bands are ClpX, ClpP, and creatine kinase.

### DETAILED DESCRIPTION OF INVENTION

The introduction of heterologous enzymes into a cell to generate novel synthetic pathways can result in a number of challenges, especially when the enzymes of those pathways compete with native enzymes for substrate. A number of recent studies have focused on experimental and theoretical advantages associated with directing metabolite flux by controlling enzyme levels using rational strain design for over-production of natural metabolites, such as gene knock-outs or promoter replacements (Anesiadis et al., ACS Synthetic Biology (2013); Callura et al., PNAS:109 (2012); Farmer and Liao, Nat. Biotechnol.: 18 (2000); Solomon et al., Metab. Eng.: 14 (2012); Soma et al., Metab. Eng.:23 (2014); Torella et al., PNAS:110 (2013)). While use of inducible promoters to turn on heterologous gene expression in E. coli through small molecule inducers or temperature change has been well developed, methods for dynamically knocking down expression of native genes are more limited. However, many of these systems have been optimized on plasmids or at relatively high expression levels, making them difficult to integrate in context with heterologous biosynthetic pathways that are already taxing to the host cells (Cardinale and Arkin, Biotechnology Journal:7 (2012)). Similarly, knock-outs of genes encoding enzymes involved in the metabolism of the cell can be detrimental to cell growth and viability.

Traditional approaches of metabolic engineering have produced strains with only a few available “control knobs,” especially with respect to changing the metabolism of the cell during the course of a fermentation. At the beginning of a fermentation, the growth phase, biomass production and expression of recombinant proteins are important, whereas in the production phase, the cell metabolite flux must be directed towards synthesis of desired compounds. Described herein is the development of methods to overcome these limitations. The methods provided herein allow for the rapid and controlled redirection of metabolites from glycolysis and production of biomass towards the production of desired compounds (e.g., heterologous compounds), by modulating the abundance of enzymes within the cell. The methods described herein are, at least in part, based on the surprising discovery that metabolites, particularly glycolytic intermediates such as glucose-6-phosphate, can be rapidly redirected in a controlled manner from glycolysis toward synthesis of desired compounds, such as myo-inositol, glucuronic acid, and/or glucaric acid, without substantially affecting the replication and growth of the cell.

Aspects of the disclosure relate to redirecting metabolites into a pathway for the production of heterologous compounds, such as myo-inositol, glucuronic acid and/or glucaric acid. The branch point between these biosynthetic pathways and central metabolism occurs in upper glycolysis at the glucose-6-phosphate node. To increase production of heterologous compounds, glycolytic intermediates, such as glucose-6-phosphate, must be directed into the biosynthetic pathways rather than into central metabolism, which may be detrimental to the viability and growth of the cell. The methods described herein provide dynamic control of the flux of glucose-6-phosphate to allow cellular growth and function of the glycolytic pathway during the “growth state,” and rapid redirection of glucose-6-phosphate in the “production state” to enhance synthesis of heterologous compounds (e.g., myo-inositol, glucuronic acid, and/or glucaric acid), thereby restricting cellular growth. Additional methods for use in the production of glucuronic acid and/or glucaric acid are disclosed, for example, in PCT Publication No. WO 2009/145838, and in U.S. Pat. No. 8,835,147, which are incorporated by reference herein in their entireties.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Aspects of the disclosure involve reducing glucose-6-phosphate entry into the pentose phosphate pathway during specific stages of fermentation. Without wishing to be bound by any particular theory, it is thought that glucose and other sugar molecules enter the cell through a sugar-specific phosphotransferase system, which imports and phosphorylates the sugar molecule (e.g., glucose-6-phosphate). Once in the cell, glucose-6-phosphate, for example, can enter the glycolytic pathway by conversion of glucose-6-phosphate to fructose-6-phosphate by the isomerase Pgi, or glucose-6-phosphate can enter the pentose phosphate pathway by conversion of glucose-6-phosphate to 6-phospho-glucono-1,5-lactone by the glucose-6-phosphate dehydrogenase, Zwf. Glucose-6-phosphate entry into the pentose phosphate pathway can be inhibited by reducing expression of the glucose-6-phosphate dehydrogenase. In some embodiments, reducing expression of the glucose-6-phosphate dehydrogenase comprises eliminating expression of the glucose-6-phosphate dehydrogenase. The expression of glucose-6-phosphate dehydrogenase can be reduced or eliminated by any method known in the art, including knocking out the gene encoding the glucose-6-phosphate dehydrogenase, mutating the coding sequence such that an inactive protein is produced, or replacing or inactivating the promoter of the gene. In some examples, the gene encoding the glucose-6-phosphate dehydrogenase is knocked-out in the cell. As used herein, a “knock-out” or “knocking out” a gene refers to removing or mutating at least a portion of the coding and/or non-coding sequence of the gene such that no functional protein is produced.

Aspects of the disclosure relate to redirecting the glucose-6-phosphate flux in a cell by regulating activity of a phosphofructokinase (Pfk). In some cells, for example, Escherichia coli, phosphofructokinase exists in two isozymes, Pfk-I and Pfk-II. In cells like E. coli, Pfk-I is attributed with the majority of observed phosphofructokinase activity in the cell. In some embodiments, Pfk-II expression is reduced in the cell. In some embodiments, Pfk-II expression is eliminated in the cell. In some embodiments, the gene encoding Pfk-II (e.g., pfkB) is knocked out in the cell. Activity of Pfk-I can be regulated to direct glucose-6-phosphate utilization. As used herein, a “Pfk-I ON state” is one in which Pfk-I protein is present in the cell and functions to catalyze the conversion of fructose-6-phosphate to fructose-1,6-biphosphate. As also used herein, a “Pfk-I OFF state” is one in which the amount of Pfk-I protein is reduced or absent in the cell. In the Pfk-I OFF state, glucose-6-phosphate does not proceed through the glycolytic pathway but rather may be redirected into other pathways, for example a pathway for the production of heterologous compounds such as myo-inositol, glucuronic acid, and/or glucaric acid. In some embodiments, a cell is in the Pfk-I ON state to allow cell growth and biomass production. In some embodiments, a cell is in the Pfk-I OFF state to restrict cell growth and biomass production and to enhance utilization of glucose-6-phosphate for the production of heterologous compounds such as myo-inositol, glucuronic acid, and/or glucaric acid.

The transition between the Pfk-I ON state and the Pfk-I OFF state can be regulated by any method known in the art or described herein. In preferred embodiments, the transition between the Pfk-I ON state and the Pfk-I OFF state is a rapid change in enzyme activity. In some embodiments, the regulation of Pfk-I activity is controlled by regulating the amount of Pfk-I in the cell. In some embodiments, the amount of Pfk-I is regulated by transcriptional control. In some embodiments, the amount of Pfk-I is regulated by translational control. In some embodiments, Pfk-I activity is regulated by degrading Pfk-I under conditions in which the Pfk-I OFF state is desired (e.g., to enhance production of heterologous compounds, such as myo-inositol). In some embodiments, Pfk-I is targeted for degradation by a protease (e.g., the ClpXP protease, ClpAP protease, proteosome). Methods of targeting proteins for degradation are well known in the art. In one example, the Pfk-I is targeted for degradation using a peptide tag and adaptor protein system. In some embodiments, Pfk-I comprises a peptide tag. In some embodiments, Pfk-I is fused to an SsrA peptide tag. In some embodiments, the SsrA tag is fused to the N-terminus of Pfk-I. In some embodiments, the SsrA tag is fused to the C-terminus of Pfk-I. In some embodiments, the sequence of the SsrA peptide tag is AANDENYALAA (SEQ ID NO: 1). The sequence of the SsrA peptide tag can be optimized to enhance the rate of degradation of the tagged protein in the presence of SspB and reduce the rate of degradation of the tagged protein in the absence of SspB (McGinness et al., Mol. Cell. 22(5): 2006). In some embodiments, the sequence of the SsrA peptide tag is AANDENYSENYADAS (SEQ ID NO: 2). Additional sequences of peptide tags that target a protein for degradation are known in the art (see, for example, Flynn et al., PNAS 98: 19, 2001; Landry et al., Appl. Environ. Microbiol. 79(8): 2013; Griffith et al., Mol. Micro. 70(4): 2008). It should be appreciated that any peptide tag that targets a protein for degradation may be compatible with aspects of the invention.

In some embodiments, the cell also expresses an adaptor protein for targeting a tagged protein for degradation. In some embodiments, the adaptor protein targets the tagged protein to the ClpXP protease for degradation. In some embodiments, the adaptor protein is SspB. Without wishing to be bound by any particular theory, it is generally appreciated that the adaptor SspB binds to a sequence within the SsrA peptide tag and promotes interaction with a cellular protease resulting in enhanced degradation of the SsrA-tagged protein. As shown in the Examples section, in some embodiments, inducing targeted degradation of Pfk-I by induction of SspB resulted in a reduction of Pfk-I activity of at least 10%, at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, including all intermediate values. In some embodiments, inducing targeted degradation of Pfk-I by induction of SspB resulted in a reduction of Pfk-I levels of at least 20%, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, including all intermediate values.

In some embodiments, expression of the adaptor protein (e.g., SspB) is under control of an inducible promoter. In the presence of an inducer that acts on the inducible promoter, the adaptor protein (e.g., SspB) is expressed and targets the degradation of Pfk-I comprising an SsrA tag. This can result in a rapid decline in Pfk-I in the cell and Pfk-I activity, thereby redirecting glucose-6-phosphate toward production of heterologous compounds such as myo-inositol, glucuronic acid, and/or glucaric acid. In some embodiments, expression of SspB is under control of an anhydrotetracycline-inducible promoter. In some embodiments, expression of SspB is under control of the Pₜₑₜ promoter. In some embodiments, the inducer is anhydrotetracycline (aTc). In some embodiments, expression of SspB is under control of an arabinose-inducible promoter and the inducer is arabinose. In some embodiments, expression of SspB is under control of a promoter that is induced in conditions of phosphate starvation, and the inducer is a condition in which the cell is starved of phosphate. It should be appreciated that any inducible promoter and corresponding inducer can be compatible with aspects of the invention.

In some embodiments, expression of the adaptor protein (e.g., SspB) is under control of an inducible promoter that is responsive to a molecule produced by the recombinant cell. In some embodiments an inducible promoter is responsive to an autoinducer (i.e., a quorum sensing molecule). As used herein, “autoinducer” or “quorum sensing molecule” refers to a signaling molecules that is produced in response to changes in cell-population density. For example, as the density of quorum sensing bacterial cells increases, the concentration of the quorum sensing molecule also increases. Without wishing to be bound by any particular theory, detection of an autoinducer or quorum sensing molecule by bacteria can act as a stimulation which can lead to altered gene expression once a threshold level has been reached. Quorum sensing molecules include, without limitation, classes of acylated homoserine lactones (e.g., 3OC6HSL), peptides and furanosyl borate disters. In some embodiments, the quorum sensing molecule is 3OC6HSL.

The quorum sensing molecule may be any quorum sensing molecule produced by any prokaryotic or eukaryotic cells. In some embodiments, the quorum sensing molecule is from a bacterial cell (e.g., Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp. and Pantoea spp.). In some embodiments the quorum sensing molecule is from a Pantoea species. In some embodiments the quorum sensing molecule is from Pantoea stewartii. It should be appreciated that any of the receptors and/or transcription factors that bind to the quorum sensing molecules, described herein, as well as any of the promoters that are responsive to the level of any of the quorum sensing molecules, described herein, are also within the scope of this disclosure.

In some embodiments, an inducible promoter is responsive to a level of a quorum sensing molecule. As used herein, an inducible promoter is “responsive to” a quorum sensing molecule if the quorum sensing molecule modulates expression of at least one nucleic acid or gene (e.g., SspB) to which the inducible promoter is operatively linked. In some embodiments, the inducible promoter increases the expression of a nucleic acid or gene when a threshold level of a quorum sensing molecule is reached. In some embodiments, the inducible promoter decreases or ceases expression of a nucleic acid or gene when a threshold level of a quorum sensing molecule is reached. In some embodiments, the inducible promoter is responsive to a level of 3-oxohexanoyl-homoserine-lactone (3OC6HSL). In some embodiments, in the presence of a quorum sensing molecule that acts on the inducible promoter, the adaptor protein (e.g., SspB) is expressed and targets the degradation of Pfk-I comprising an SsrA tag. This can result in a rapid decline in Pfk-I in the cell and decline in Pfk-I activity, thereby redirecting glucose-6-phosphate toward production of heterologous compounds such as myo-inositol, glucuronic acid, and/or glucaric acid. In some embodiments, expression of SspB is under control of a quorum sensing molecule inducible promoter. In some embodiments, expression of SspB is under control of a 3-oxohexanoyl-homoserine-lactone (3OC6HSL) inducible promoter. In some embodiments, expression of SspB is under control of the PeasS or PeasR promoter. It should be appreciated that any inducible promoter and corresponding inducer can be compatible with aspects of the invention.

Further aspects of the disclosure relate to expressing a myo-inositol-1-phosphate synthase in a cell. As would be appreciated in the art, any protein with myo-inositol-1-phosphate synthase activity can be compatible with aspects of the present disclosure. In some embodiments, the gene encoding the myo-inositol-1-phosphate synthase is a Saccharomyces gene. Expression of the myo-inositol-1-phosphate synthase can be under control of a constitutively active promoter or an inducible promoter. In some embodiments, the myo-inositol-1-phosphate synthase is under control of an inducible promoter. In some embodiments, the inducer for induction of expression of the myo-inositol-1-phosphate synthase is the same inducer as used for induction of expression of SspB. In some embodiments, the inducer for induction of expression of the myo-inositol-1-phosphate synthase is a different inducer than that as used for induction of expression of SspB. In some embodiments, the inducer is isopropyl-β-D-1-thiogalactopyranoside (IPTG).

In some embodiments, myo-inositol-1-phosphate is converted into myo-inositol through the activity of an endogenous phosphatase in the cell. In some embodiments, the cell may be engineered to express a phosphatase for the conversion of myo-inositol-1-phosphate is converted into myo-inositol.

As would be understood by one of ordinary skill in the art, any inducer molecule compatible for inducing expression from a particular promoter may be used. The effective concentration of the inducer, such as aTc or IPTG, can vary depending on the method and level of induction desired. Optimal concentration of the inducer, for a given application, can be determined without undue experimentation. For example, in some embodiments, the inducer is added at approximately 0.1, 0.5, 1, 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or more than 990 ng/ml including all intermediate values.

The timing of induction is also a parameter that can be optimized as would be understood by one of ordinary skill in the art. In some embodiments, a single inducer concentration is added to the cell culture at a single time point. In other embodiments, the inducer can be added multiple times, either at the same concentration each time or at different concentrations. In some embodiments, a first inducer added to induce expression of SspB and a second inducer is used to induce expression of the myo-inositol-1-phosphate synthase. In some embodiments, the first inducer and second inducer is the same inducer. In some embodiments, the first inducer and the second inducer are difference inducers. In some embodiments, the first inducer is added to the culture before the second inducer. In some embodiments, the second inducer is added to the culture before the first inducer. In some embodiments, the first inducer and the second inducer are added to the culture at substantially the same time.

According to aspects of the invention, recombinant cells that express one or more enzymes associated with the invention and the use of such cells in producing heterologous compounds (e.g., myo-inositol, glucuronic acid, glucaric acid), are provided. It should be appreciated that genes encoding each of the enzymes associated with the invention can be obtained from a variety of sources, including, without limitation, a microbial cell or a eukaryotic cell. In some embodiments, the gene encoding the Pfk-1 and/or the gene encoding SspB is obtained from a strain of Escherichia coli, such as E. coli M1655. In some embodiments, the gene encoding the myo-inositol-1-phosphate synthase, is obtained from a yeast strain such as a strain of Saccharomyces. In some embodiments, the strain of Saccharomyces is S. cerevisiae. In some embodiments, the gene encoding the myo-inositol oxygenase is a plant gene, a mammalian gene, a fungal gene, such as a yeast gene, an arthropod gene, or a bacterial gene. In some embodiments, the gene encoding the myo-inositol oxygenase is a obtained from a mammal, such as a mouse. In some embodiments, the gene encoding the myo-inositol oxygenase is a plant gene, a mammalian gene, a fungal gene, such as a yeast gene, an arthropod gene, or a bacterial gene. In some embodiments, the gene encoding the uronate dehydrogenase is obtained from a bacterium, such as Pseudomonas syringae, Pseudomonas putida, or Agrobacterium tumefaciens. In some embodiments, the gene encoding the uronate dehydrogenase is a plant gene, a mammalian gene, a fungal gene, such as a yeast gene, an arthropod gene, or a bacterial gene. It should be appreciated that any of the nucleic acids and/or polypeptides described herein can be codon-optimized and expressed recombinantly in a codon-optimized form.

As would be appreciated by one of ordinary skill in the art, genes that are homologous to the genes expressed according to aspects of the invention could be obtained from other species and could be identified by homology searches, for example through a protein BLAST search, available at the National Center for Biotechnology Information (NCBI) internet site (ncbi.nlm.nih.gov). Genes associated with the invention can be amplified from DNA from any source of DNA which contains the given gene, such as using polymerase chain reaction (PCR) amplification. In some embodiments, genes associated with the invention are synthetic. Any means of obtaining a gene encoding enzymes associated with the invention are compatible with the instant invention.

The invention involves recombinant expression of genes encoding enzymes discussed above, functional modifications and variants of the foregoing, as well as uses relating thereto. Homologs and alleles of the nucleic acids associated with the invention can be identified by conventional techniques. The skilled artisan also is familiar with the methodology for screening cells and libraries for expression of such molecules which then are routinely isolated, followed by isolation of the pertinent nucleic acid molecule and sequencing.

In general, homologs and alleles typically will share at least 75% nucleotide identity and/or at least 90% amino acid identity to the sequences of nucleic acids and polypeptides, respectively, in some instances will share at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% nucleotide identity and/or at least 90%, 91%, 92%, 93%, 94%, 95%, 96%, 97%, 98% or 99% amino acid identity. The homology can be calculated using various, publicly available software tools developed by NCBI (Bethesda, Md.) that can be obtained through the NCBI internet site. Exemplary tools include the BLAST software, also available at the NCBI internet site (www.ncbi.nlm.nih.gov). Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention.

The invention also includes degenerate nucleic acids which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code. The invention also embraces codon optimization to suit optimal codon usage of a host cell.

The invention also provides modified nucleic acid molecules which include additions, substitutions and deletions of one or more nucleotides. In preferred embodiments, these modified nucleic acid molecules and/or the polypeptides they encode retain at least one activity or function of the unmodified nucleic acid molecule and/or the polypeptides, such as enzymatic activity. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art. The term “stringent conditions” as used herein refers to parameters with which the art is familiar. Nucleic acid hybridization parameters may be found in references which compile such methods, e.g. Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York. More specifically, stringent conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH2PO4(pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, the membrane upon which the DNA is transferred is washed, for example, in 2×SSC at room temperature and then at 0.1-0.5×SSC/0.1×SDS at temperatures up to 68° C. There are other conditions, reagents, and so forth which can be used, which result in a similar degree of stringency. The skilled artisan will be familiar with such conditions, and thus they are not given here.

For example, modified nucleic acid molecules which encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules which encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

The invention embraces variants of polypeptides. As used herein, a “variant” of a polypeptide is a polypeptide which contains one or more modifications to the primary amino acid sequence of the polypeptide. Modifications which create a variant can be made to a polypeptide, for example, 1) to reduce or eliminate an activity of a polypeptide; 2) to enhance a property of a polypeptide; 3) to provide a novel activity or property to a polypeptide, such as addition of an antigenic epitope or addition of a detectable moiety; or 4) to provide equivalent or better binding between molecules (e.g., an enzymatic substrate). Modifications to a polypeptide are typically made to the nucleic acid which encodes the polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin, addition of a fatty acid, and the like. Modifications also embrace fusion proteins comprising all or part of the amino acid sequence.

One of ordinary skill in the art will be familiar with methods for predicting the effect on protein conformation of a change in protein sequence, and can thus “design” a variant of a polypeptide according to known methods. One example of such a method is described by Dahiyat and Mayo in Science 278:82 87, 1997, whereby proteins can be designed de novo. The method can be applied to a known protein to vary a only a portion of the polypeptide sequence. By applying the computational methods of Dahiyat and Mayo, specific variants of a polypeptide can be proposed and tested to determine whether the variant retains a desired conformation

In general, variants include polypeptides which are modified specifically to alter a feature of the polypeptide unrelated to its desired physiological activity. For example, cysteine residues can be substituted or deleted to prevent unwanted disulfide linkages. Similarly, certain amino acids can be changed to enhance expression of a polypeptide by eliminating proteolysis by proteases in an expression system (e.g., dibasic amino acid residues in yeast expression systems in which KEX2 protease activity is present).

Mutations of a nucleic acid which encode a polypeptide preferably preserve the amino acid reading frame of the coding sequence, and preferably do not create regions in the nucleic acid which are likely to hybridize to form secondary structures, such a hairpins or loops, which can be deleterious to expression of the variant polypeptide.

Mutations can be made by selecting an amino acid substitution, or by random mutagenesis of a selected site in a nucleic acid which encodes the polypeptide. Variant polypeptides are then expressed and tested for one or more activities to determine which mutation provides a variant polypeptide with the desired properties. Further mutations can be made to variants (or to non-variant polypeptides) which are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of a gene or cDNA clone to enhance expression of the polypeptide. The activity of variant polypeptides can be tested by cloning the gene encoding the variant polypeptide into a bacterial or eukaryotic expression vector, introducing the vector into an appropriate host cell, expressing the variant polypeptide, and testing for a functional capability of the polypeptides as disclosed herein.

The skilled artisan will also realize that conservative amino acid substitutions may be made in polypeptides to provide functionally equivalent variants of the foregoing polypeptides, i.e., the variants retain the functional capabilities of the polypeptides. As used herein, a “conservative amino acid substitution” refers to an amino acid substitution which does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York. Exemplary functionally equivalent variants of polypeptides include conservative amino acid substitutions in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In general, it is preferred that fewer than all of the amino acids are changed when preparing variant polypeptides. Where particular amino acid residues are known to confer function, such amino acids will not be replaced, or alternatively, will be replaced by conservative amino acid substitutions. Preferably, 1, 2, 3, 4, 5, 6, 7, 8, 9, 10, 11, 12, 13, 14, 15, 16, 17, 18, 19, 20 residues can be changed when preparing variant polypeptides. It is generally preferred that the fewest number of substitutions is made. Thus, one method for generating variant polypeptides is to substitute all other amino acids for a particular single amino acid, then assay activity of the variant, then repeat the process with one or more of the polypeptides having the best activity.

Conservative amino-acid substitutions in the amino acid sequence of a polypeptide to produce functionally equivalent variants of the polypeptide typically are made by alteration of a nucleic acid encoding the polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a polypeptide.

The invention encompasses any type of cell including prokaryotic and eukaryotic cells. In some embodiments the cell is a bacterial cell, such as Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp. and Pantoea spp. The bacterial cell can be a Gram-negative cell such as an Escherichia coli (E. coli) cell, or a Gram-positive cell such as a species of Bacillus. In some embodiments, the cell is an E. coli MG1655 cell.

In other embodiments the cell is a fungal cell such as yeast cells, e.g., Saccharomyces spp., Schizosaccharomyces spp., Pichia spp., Paffia spp., Kluyveromyces spp., Candida spp., Talaromyces spp., Brettanomyces spp., Pachysolen spp., Debaryomyces spp., Yarrowia spp. and industrial polyploid yeast strains. In some embodiments, the yeast strain is a S. cerevisiae strain. Other examples of fungi include Aspergillus spp., Pennicilium spp., Fusarium spp., Rhizopus spp., Acremonium spp., Neurospora spp., Sordaria spp., Magnaporthe spp., Allomyces spp., Ustilago spp., Botrytis spp., and Trichoderma spp. In other embodiments the cell is an algal cell, a plant cell, or a mammalian cell.

It should be appreciated that some cells compatible with the invention may express an endogenous copy of one or more of the genes associated with the invention as well as a recombinant copy. In some embodiments if a cell has an endogenous copy of one or more of the genes associated with the invention then the methods will not necessarily require adding a recombinant copy of the gene(s) that are endogenously expressed. In some embodiments the cell may endogenously express one or more enzymes from the pathways described herein and may recombinantly express one or more other enzymes from the pathways described herein, including pathways for the production of myo-inositol or for the production of glucuronic acid and/or glucaric acid. It should be appreciated that the principles of redirecting metabolite flux in order to produce molecules such as myo-inositol, glucuronic acid and/or glucaric acid are compatible with multiple cell types. The specific aspects of the metabolite redirecting can be optimized as appropriate for different cell types.

In some embodiments, one or more of the genes associated with the invention is integrated into the genome of the cell. In some embodiments, one or more genes may be integrated into a phage attachment site on the genome of the cell (e.g., the HK022 phage attachment site in E. coli M1655). Genes can be integrated into their native locus or can be integrated into a locus that is not their native locus. For example, in some embodiments described herein, pfkA is integrated into its native locus with the native promoter replaced by a synthetic one. In some embodiments, an inducible cassette, such as an aTc-inducible SspB cassette is integrated into a phage attachment site, such as the HK022 phage attachment site.

In some embodiments, one or more of the genes associated with the invention is expressed in a recombinant expression vector. As used herein, a “vector” may be any of a number of nucleic acids into which a desired sequence or sequences may be inserted by restriction and ligation for transport between different genetic environments or for expression in a host cell. Vectors are typically composed of DNA although RNA vectors are also available. Vectors include, but are not limited to: plasmids, fosmids, phagemids, virus genomes and artificial chromosomes.

A cloning vector is one which is able to replicate autonomously or integrated in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host cell such as a host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the enzymes associated with the invention is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Fourth Edition, Cold Spring Harbor Laboratory Press, 2012 or Current Protocols in Molecular Biology, F. M. Ausubel, et al., eds., John Wiley & Sons, Inc., New York. Cells are genetically engineered by the introduction into the cells of heterologous DNA (RNA). That heterologous DNA (RNA) is placed under operable control of transcriptional elements to permit the expression of the heterologous DNA in the host cell. Heterologous expression of genes associated with the invention, for example for production of myo-inositol, glucuronic acid, and/or glucaric acid, is demonstrated in the Examples section using E. coli. The methods described herein for directing metabolite flux in a cell are also compatible with other bacterial cells and the concept can also be extended to non-bacterial cells.

A nucleic acid molecule that encodes an enzyme associated with the invention can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome. In some embodiments, one or more of the genes associated with the invention is integrated into the genome of the cell. In some embodiments, one or more genes may be integrated into a phage attachment site on the genome of the cell (e.g., the HK022 phage attachment site in E. coli).

In some embodiments, one or more genes associated with the invention is expressed recombinantly in a bacterial cell. Bacterial cells according to the invention can be cultured in media of any type (rich or minimal) and any composition. As would be understood by one of ordinary skill in the art, routine optimization would allow for use of a variety of types of media, including sugar sources. In some embodiments, the cells are cultured in the presence of glucose in the culture media. In some embodiments, the cells are cultured in the presence of arabinose in the culture media. In some embodiments, the cells are cultured in the presence of xylose in the culture media. In some embodiments, the cells are cultured in the presence of any combination of two or more sugars (e.g., glucose, xylose, arabinose). The selected medium can be supplemented with various additional components. Some non-limiting examples of supplemental components include glucose, amino acids, antibiotics, aTc for gene induction, IPTG for gene induction, ATCC Trace Mineral Supplement, and inducers such as aTc, according to aspects of the invention. Similarly, other aspects of the medium, and growth conditions of the cells of the invention can be optimized through routine experimentation. For example, pH, temperature, and concentration and timing of induction are non-limiting examples of factors which can be optimized.

In some embodiments, factors such as choice of media, media supplements, and temperature can influence production levels of metabolites such as myo-inositol, glucuronic acid and/or glucaric acid. In some embodiments the concentration and amount of a supplemental component such as an inducer can be optimized. For example, how often the media is supplemented with one or more supplemental components such as one or more inducers, and the amount of time that the media is cultured before harvesting the desired compound can be optimized.

Aspects of the invention relate to redirecting glucose-6-phosphate from glycolysis and pathways for the production biomass towards to heterologous pathways in a cell. As would be understood by one or ordinary skill in the art, the methods could be used to produce or increase production of any metabolite for which glucose-6-phosphate is a substrate. In some embodiments, the metabolite is myo-inositol. In some embodiments, the metabolite is glucuronic acid and/or glucaric acid. As presented in the Examples section, in some embodiments, practice of the methods described herein produced at least 2-fold more myo-inositol in a sample in which degradation of Pfk-I was induced than in an uninduced sample.

In some embodiments, the methods provided herein result in production of myo-inositol. In some embodiments, the yield of myo-inositol is at least 50, 100, 200, 300, 400, 500, 600, 700, 800, 900, at least 1000 mg L⁻¹, including all intermediate values. The titer produced for a given product will be influenced by multiple factors including choice of media, choice of promoters and inducers, and level of induction.

In some embodiments, the methods provided herein result in production of glucuronic acid. In such an embodiment, the cell can be engineered to express a gene encoding a myo-inositol oxygenase (MIOX) to convert myo-inositol to glucuronic acid. In some embodiments, the yield of glucuronic acid is at least 50, 100, 200, 300, 400, 500, 600, 700, 800, 900, 1000, 1100, at least 1200 mg L⁻¹, including all intermediate values. The titer produced for a given product will be influenced by multiple factors including choice of media, choice of promoters and inducers, and level of induction. Methods for production of glucuronic acid are disclosed, for example, in PCT Publication No. WO 2009/145838, and in U.S. Pat. No. 8,835,147, which are incorporated by reference herein in their entireties.

In some embodiments, the methods provided herein result in production of glucaric acid. In such an embodiment, the cell can be engineered to express a gene encoding a myo-inositol oxygenase (MIOX) to convert myo-inositol to glucuronic acid, and a gene encoding a uronate dehydrogenase (udh) to cover glucuronic acid to glucaric acid. In some embodiments, the yield of glucaric acid is at least 50, 100, 200, 300, 400, 500, 600, 700, 800, 900, at least 1000 mg L⁻¹, including all intermediate values. The titer produced for a given product will be influenced by multiple factors including choice of media, choice of promoters and inducers, and level of induction. Methods for production of glucaric acid are disclosed in, for example, in PCT Publication No. WO 2009/145838, and in U.S. Pat. No. 8,835,147, which are incorporated by reference herein in their entireties.

The liquid cultures used to grow cells associated with the invention can be housed in any of the culture vessels known and used in the art. In some embodiments large scale production in an aerated reaction vessel such as a stirred tank reactor can be used to produce large quantities of a desired product such as myo-inositol, glucuronic acid and/or glucaric acid.

In any of the methods described herein, the method may further involve recovering the desired compound (e.g., myo-inositol, glucuronic acid, glucaric acid) from the cell culture or the supernatant from the cell culture. Methods of recovering, isolating, and/or purifying the desired compound (e.g., myo-inositol, glucuronic acid, glucaric acid) are well known in the art.

Aspects of the invention include strategies to optimize production of desired compounds such as myo-inositol, glucuronic acid and/or glucaric acid from a cell. Optimized production of a compound refers to producing a higher amount of a compound following pursuit of an optimization strategy than would be achieved in the absence of such a strategy. One strategy for optimization is to increase expression levels of one or more genes associated with the invention through selection of appropriate promoters and ribosome binding sites. In some embodiments this may include the selection of high-copy number plasmids, or low or medium-copy number plasmids. The step of transcription termination can in some instances also be targeted for regulation of gene expression, through the introduction or elimination of structures such as stem-loops.

In some embodiments it may be advantageous to use a cell that has been optimized for production of the desired compounds such as myo-inositol, glucuronic acid and/or glucaric acid. In some embodiments, screening for mutations that lead to enhanced production of compounds such as myo-inositol, glucuronic acid and/or glucaric acid may be conducted through a random mutagenesis screen, or through screening of known mutations. In some embodiments, shotgun cloning of genomic fragments can be used to identify genomic regions that lead to an increase in production of compounds such as myo-inositol, glucuronic acid and/or glucaric acid, through screening cells or organisms that have these fragments for increased production compounds such as myo-inositol, glucuronic acid and/or glucaric acid. In some embodiments, one or more mutations can be combined in the same cell or organism.

Optimization of production of compounds such as myo-inositol, glucuronic acid and/or glucaric acid can involve optimizing selection of bacterial strains for expression of recombinant pathways described herein. In some embodiments, use of a bacterial strain that is close to wild-type, meaning a strain that has not been substantially genetically modified, may lead to increased titers of compounds such myo-inositol, glucuronic acid and/or glucaric acid.

Optimization of protein expression may also require in some embodiments that a gene encoding an enzyme be modified before being introduced into a cell such as through codon optimization for expression in a bacterial cell. Codon usages for a variety of organisms can be accessed in the Codon Usage Database (kazusa.or.jp/codon/).

In some embodiments, protein engineering can be used to optimize expression or activity of one or more enzymes associated with the invention. In certain embodiments a protein engineering approach could include determining the three dimensional (3D) structure of an enzyme or constructing a 3D homology model for the enzyme based on the structure of a related protein. Based on 3D models, mutations in an enzyme can be constructed and incorporated into a cell or organism, which could then be screened for an increased production compounds such as myo-inositol, glucuronic acid and/or glucaric acid. In some embodiments production of compounds such as myo-inositol, glucuronic acid and/or glucaric acid in a cell could be increased through manipulation of enzymes that act in the same pathway as the enzymes associated with the invention. For example in some embodiments it may be advantageous to increase expression of an enzyme or other factor that acts upstream of a target enzyme such as an enzyme associated with the invention. This could be achieved by over-expressing the upstream factor using any standard method.

Methods and compositions described herein redirecting metabolite flux in a cell have widespread applications. For pathways where glucose-6-phosphate is a substrate for production, the methods provided herein allow for rapid redirection of the substrate away from glycolysis and pathways for the production of biomass and towards pathways for production of desired compounds, such as myo-inositol, glucuronic acid and/or glucaric acid. The system could be generalized to other applications, such as balancing flux between glycolysis and the pentose phosphate pathway in response to cellular demand for NADPH, when expressing pathways with high cofactor requirements, such as fatty acid biosynthesis.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### Examples

### Example 1: Dynamic Control of Metabolism for Redirecting Fluxes of Primary Metabolites

### Abstract

Control of native enzyme levels can be a factor when optimizing strains for overproduction of heterologous compounds. However, for many central metabolic enzymes, static knockdown results in poor growth and protein expression. Provided herein is a strategy for dynamically modulating the abundance of native enzymes within a host cell using a model system for myo-inositol production from glucose. This system relies on controlled degradation of a key glycolytic enzyme, phosphofructokinase-I (Pfk-I). Through tuning Pfk-I levels, an E. coli strain with a growth mode close to wild type and a production mode with an increased glucose-6-phosphate pool available for conversion into myo-inositol was developed. The switch to production mode can be trigged by inducer addition, allowing yield, titer, and productivity to be managed through induction time. By varying the time of Pfk-I degradation, a two-fold improvement in yield and titers of myo-inositol was achieved.

### Introduction

The introduction of heterologous enzymes into a microbial host to generate novel synthetic pathways poses a number of challenges, especially when the enzymes in those pathways compete with native enzymes for substrate. To counter this problem, common strategies utilized in rational strain design for overproduction of natural metabolites, such as gene knock-outs or promoter replacements, have typically been used (Lee et al., 2012; Tyo et al., 2010; Woolston et al., 2013). However, these approaches produce strains with only a few available control points, especially with respect to changing the cell's own metabolism during the course of a fermentation. The ideal flux balance for the production phase of a fermentation differs from the flux balance required at the beginning of a fermentation, when biomass production and expression of recombinant proteins are most important.

To overcome these limitations, a number of recent works have focused on experimental and theoretical advantages associated with redirecting flux in central metabolism through dynamic control of enzyme levels (Anesiadis et al., 2013; Callura et al., 2012; Farmer and Liao, 2000; Solomon et al., 2012b; Soma et al., 2014; Torella et al., 2013). While the use of inducible promoters to turn on heterologous gene expression in E. coli through small molecule inducers or temperature change has been well developed, methods for dynamically knocking down expression of native genes are more limited. The genetic devices developed in the context of synthetic biology offer a number of possible ways to achieve gene regulation in response to extracellular and intracellular conditions (Holtz and Keasling, 2010). However, many of these systems have been optimized on plasmids or at relatively high expression levels, making them difficult to integrate in context with heterologous biosynthetic pathways that are already taxing to the host cells (Cardinale and Arkin, 2012). Dynamic control systems which could be integrated into production strains with minimal change in baseline performance would provide a valuable advantage in microbial production of chemicals.

A node for controlling fluxes in primary metabolism could be the metabolic branch point at glucose-6-phosphate (G6P). G6P can be routed into native metabolism through both glycolysis and the oxidative pentose phosphate pathway, as well as into heterologous production of myo-inositol via INO1 from Saccharomyces cerevisiae (Hansen et al., 1999). Myo-inositol can be further converted into other useful products, such as glucaric acid, a biopolymer precursor (Werpy and Petersen, 2004) and scyllo-inositol, which has been studied as a therapeutic for Alzheimers (Yamaoka et al., 2011). The pathway for glucaric acid has already been demonstrated in E. coli (Moon et al., 2009) and theoretical yields of near 100% are possible; however, G6P must be directed into this pathway at the expense of central metabolism. Previous studies have focused on controlling the G6P utilization in glycolysis versus the pentose phosphate pathway (Callura et al., 2012), but dynamic redirection of G6P into a heterologous pathway has not been demonstrated. Accordingly, described herein is a method to direct G6P into myo-inositol production and restrict biomass formation by controlling phosphofructokinase (Pfk-I) levels. Redirecting primary metabolism poses unique challenges, as heterologous pathway enzymes are often selected from secondary metabolism or may be acting on non-native substrates, while the central metabolic enzymes utilize primary metabolites very efficiently. Global studies have indicated central metabolic enzymes typically have a higher catalytic efficiency than enzymes in secondary metabolism and are likely to be operating on a substrate pool near the KM value of the enzyme (Bar-Even et al., 2011; Bennett et al., 2009).

To minimize lag time associated with dilution of stable proteins and generate a quick response as well as large dynamic range, post-translational control of Pfk through use of modified SsrA tags was implemented. A number of SsrA tag variants have been reported which alter the half-life of the tagged protein or have varying degradation rates dependent on the presence and absence of SspB, an adaptor protein that tethers target proteins to ClpXP (Andersen et al., 1998; Davis et al., 2011; McGinness et al., 2006). By appending such a tag to the coding sequencing of Pfk-I and knocking out the native copy of sspB, the half-life of Pfk-I could be controlled through expression of SspB from an inducible promoter. This strategy allowed rapid changes in the steady-state level of Pfk-I to be achieved. Using this system in E. coli, increases in both titer and yield of myo-inositol (MI), a precursor in glucaric acid production were achieved.

### Selection of Phosphofiuctokinase-1 as a Control Point for Glucose-6-Phosphate Flux

A dynamic control strategy is applied herein to the redirection of flux into a pathway for the production of D-glucaric acid from glucose. The branch point between this pathway and central metabolism occurs in upper glycolysis at the glucose-6-phosphate (G6P) node. To maximize glucaric acid production, G6P can be directed into this pathway rather than into central metabolism. However, redirection of G6P into a heterologous pathway has not previously been demonstrated. Control of phosphofructokinase (Pfk-I) levels was evaluated here as a method to direct G6P into glucaric acid production and restrict biomass formation.

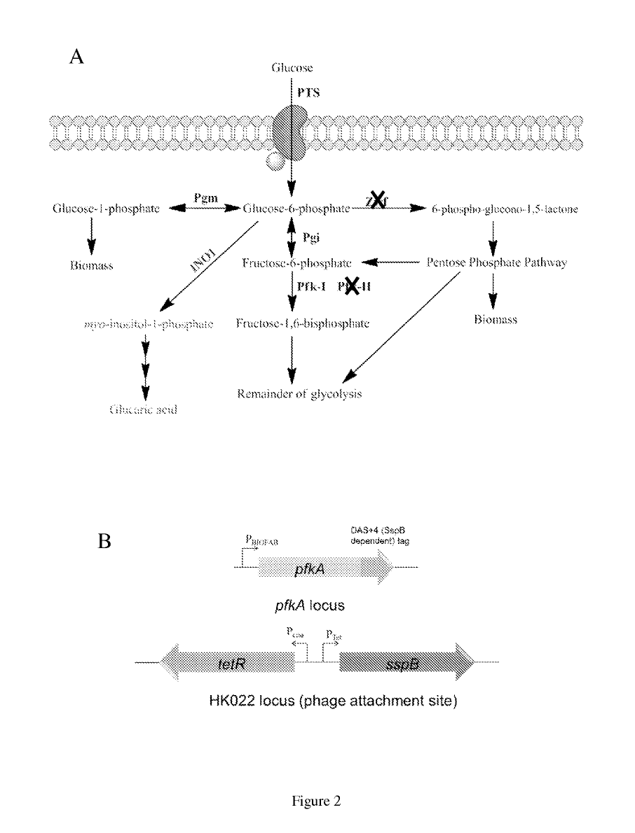

The primary native enzymes acting on the branch point metabolite G6P are phosphoglucose isomerase (Pgi) and glucose-6-phosphate dehydrogenase (Zwf) (FIG. 2A). Although knockout of these enzymes will result in a strain that cannot consume G6P (Shiue et al.) they are not necessarily appropriate targets for dynamic control. The interconversion between G6P and fructose-6-phosphate (F6P) catalyzed by Pgi is near equilibrium within the cell, indicating that the enzyme may not exert significant control over flux (Stephanopoulos et al, 1998). Previous reports from in vitro simulation of glycolysis indicate that Pfk levels controlled the utilization of G6P (Delgado et al., 1993).

To analyze this in E. coli, a model of G6P consumption was constructed, using available in vitro kinetic data and published information on steady-state flux distribution, intracellular metabolite levels, and cofactor levels during growth on glucose. This initial model was used to explore inherent kinetic limitations, but did not account for changes in downstream regulation, glucose uptake rate, and cofactor pools, which occur as the levels of glycolytic enzymes are varied.

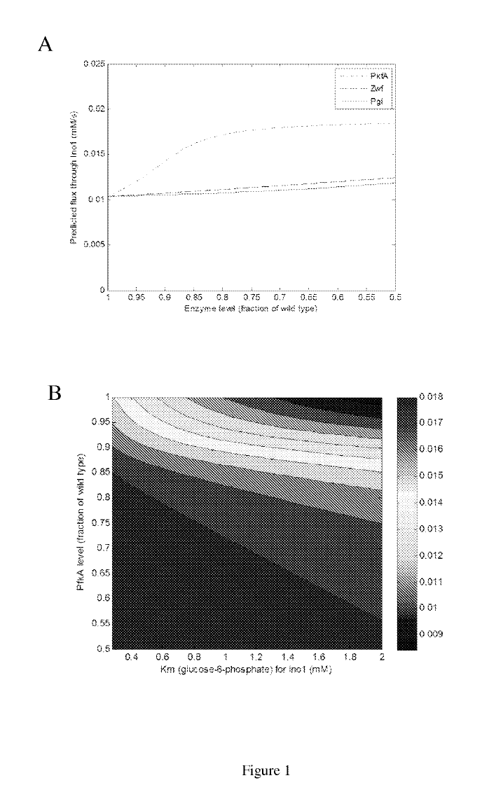

The reaction catalyzed by IN01, myo-inositol-1-phosphate synthase from S. cerevisiae, was included as the first step in the pathway for the production of glucaric acid (Moon et al., 2009). The model was used to predict steady-state rate of G6P consumption by IN01. FIG. 1A illustrates the predicted flux through INO1 as a function of enzyme level knockdown. 25% knockdown of Pfk is predicted to increase flux through INO1 nearly two-fold, while almost complete knockdown of Pgi would be required to achieve this increase in flux (FIG. 1A). The increase in flux can depend on the Kₘ value of IN1, which has been estimated for G6P in vitro to be 1.18 mM, indicating that increases in substrate concentration could still be expected to result in increased conversion rate (Majumder et al., 1997). Sensitivity analysis (FIG. 1B) illustrates that flux improvements would be possible over a range of Kₘ values for IN01. Based on the predicted increases in flux entering the glucaric acid pathway, Pfk was selected for further development as a control point.

### Control of Glucose Flux and Growth Rate Via Controlled Degradation of Pfk-I

With Pfk as the target for control, a strain background was developed that would allow the effect of Pfk knockdown to be observed (FIG. 2A). Pfk exists as two isozymes in E. coli, and the major form, Pfk-I, accounts for more than 90% of the observed activity (Keseler et al., 2011). Pfk-I was selected as the target enzyme for control, while the isozyme Pfk-11 was eliminated by knockout of pfkB. Additionally, zwf was knocked out to eliminate G6P flux into the pentose phosphate pathway, generating E. coli strain IB1379. These knockouts resulted in a 5% reduction in growth rate on M9 glucose minimal medium relative to the parent E. coli strain IB531.

For achieving dynamic knockdown of Pfk-I, a system based on controlled protein degradation was utilized. The coding sequence of pfkA was altered by appending a modified SsrA tag to the 3′ end of the gene. SsrA tagging of a protein results in rapid degradation of the target protein in the presence of the native E. coli adaptor protein SspB, but slow degradation in the absence of SspB (McGinness et al., 2006). Additionally, native regulation of Pfk-I expression was disrupted by replacement of the native promoter sequence, which contains a binding site for the transcription factor Cra, with a constitutive promoter selected from the BIOFAB modular library (Mutalik et al., 2013). Six promoters from this library were tested, and the one resulting in Pfk activity closest to the wild-type level was selected. To make the default state “ON” (slow degradation of Pfk-1), SspB was knocked out at its native locus. Control was initially tested by expression through aTc-inducible expression of SspB from the plasmid pKVS-SspB. This did not result in complete growth arrest in the “OFF” state, possibly due to plasmid loss or instability. To improve system performance, the anhydrotetracycline expression cassette for SspB was integrated into the genome at the phage attachment site, HK022, resulting in much better dynamic range of the system (FIG. 2B).

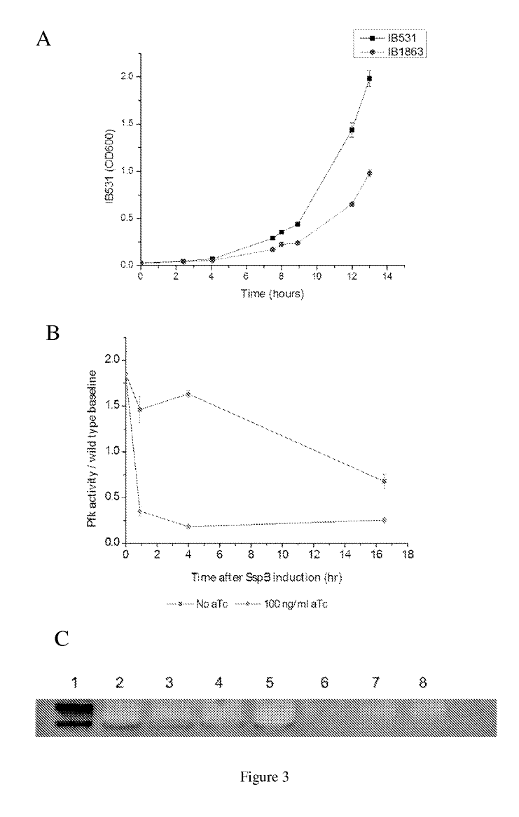

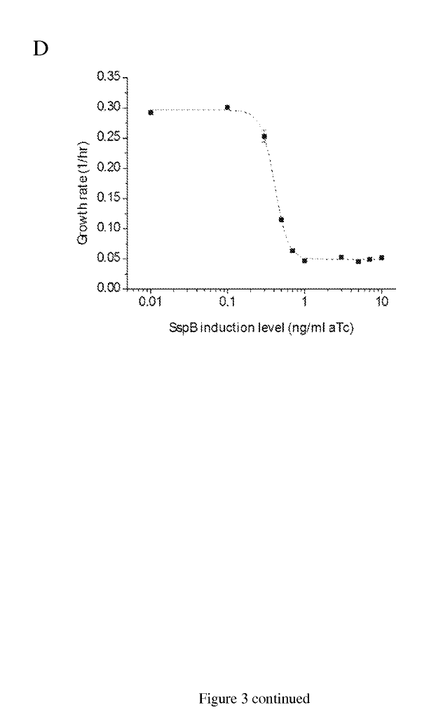

The resultant E. coli strain, IB1863, showed a 15% reduction in growth rate relative to 1B531 on minimal medium (FIG. 3A). Both strains maintained a similar growth profile and no effect was seen on the final OD reached by the culture. The baseline Pfk activity in E. coli strain 1B1863 in the absence of SspB was 1.8× higher than that observed in E. coli strain IB531. However, upon addition of anhydrotetracycline (aTc) to induce expression of SspB from the P_{L-tetO-1} promoter, Pfk-I activity in E. coli strain 1B1863 declined very rapidly (FIG. 3B), decreasing to only 35% of wild-type within one hour of SspB induction and 18% of wild-type after 4 hours. The decline in activity corresponded with observed growth arrest in the aTc induced flasks, which showed over 90% reduction in growth rate. The growth arrest and activity reduction were stable over the 16 hour time period tested. Western blotting with anti-Pfk-I showed consistent levels of the protein over 16 hours in untreated IB1863, while Pfk-I levels were below the limit of detection for cultures treated with aTc (FIG. 3C). Overexpression of SspB from pKVS-SspB was also tested in a sspB deficient background in the absence of tagged Pfk-I and no change in activity was seen, confirming inducible SspB overexpression alone had no effect. These results indicated that Pfk-1 degradation can be controlled through SspB induction and this can be used to alter cellular phenotype.

The degree of growth arrest was modulated through titration of inducer (FIG. 3D), allowing the entire dynamic range to be utilized, resulting in growth rate between 0.05 hr⁻¹ and 0.30 hr⁻¹ at 30° C. The system shows full induction at an aTc concentration of 1 ng/ml. Previous characterization of the Pᵤₑₜ₋ₒ promoter in a plasmid context indicated that 10 ng/ml of aTc was required for full induction (Lutz and Bujard, 1997). The relative change is likely due to differences in the amount of TetR produced through genomic versus plasmid-based expression. At a concentration of 1 ng/ml, there are 1.4×10¹⁵ molecules/L of aTc available in the medium. Given a biomass concentration of 10⁹ cells/L (approximately OD 1), this only provides enough aTc molecules to bind to 500 TetR molecules per cell, likely below the level produced from plasmid-based expression. The affinity of TetR for aTc is also well below the concentrations added to the medium, with the K_{D} for [aTc-Mg]₂⁺ binding reported as 8×10⁻¹³M (Kamionka et al., 2004).

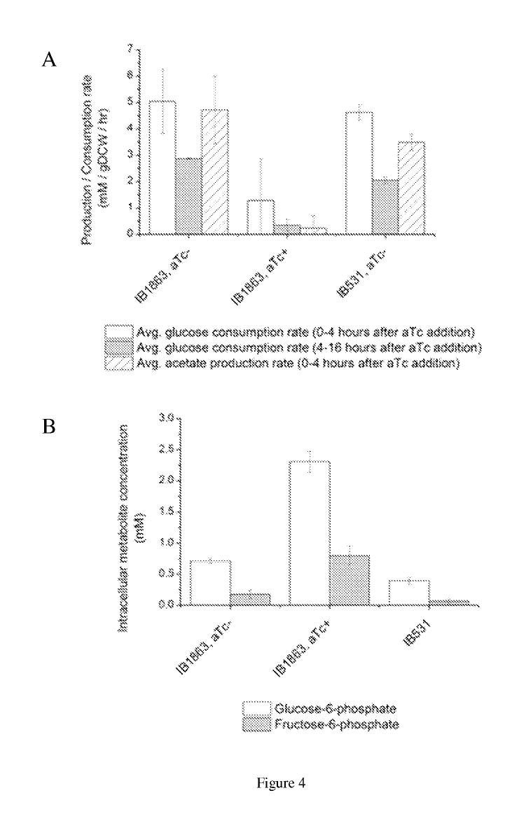

Glucose uptake and acetate production rates were also measured in IB1863. In the ON state, IB1863 showed a profile almost identical to IB531, but as expected in the OFF state, glucose uptake was greatly reduced (FIG. 4A). Acetate production was also lower, indicative of reduced flux into lower glycolysis. Additionally, intracellular levels of glucose-6-phosphate and fructose-6-phosphate were elevated in the OFF state due to limited flux into lower glycolysis (FIG. 4B). Based on the thermodynamics of the G6P to F6P interconversion, it is expected that the sugar phosphate pool will be approximately 67% G6P and 33% F6P at equilibrium (Stephanopoulos et al., 1998). The intracellular metabolite measurements in IB1863 showed a pool of 75% G6P and 25% F6P for Pfk-I knockdown and 80% G6P and 20% F6P for the control condition, consistent with the expectation that the reaction catalyzed by Pgi is near equilibrium.

### Application of the Pfk-I Valve to Myo-Inositol Production

Strains IB1863 and IB531 were transformed with pTrc-INO1, enabling IPTG-inducible expression of INO1, which catalyzes the conversion of G6P to myo-inositol-1-phosphate (MI1P). MI1P was then converted to MI by an endogenous phosphatase in E. coli (Hansen et al., 1999; Moon et al., 2009). In all cultures, INO1 expression was induced at inoculation through addition of 50 μM IPTG. Fermentations were carried out for 78 hours in shake flasks containing modified MOPS minimal medium and 10 g/L glucose. MI titers were assayed at the conclusion of the experiment. Switching of IB1863 between growth (Pfk-I ON) and production (Pfk-I OFF) modes was controlled through addition of 100 ng/ml aTc to induce SspB at t=0, 11.5, 18, 32, and 47 hours.

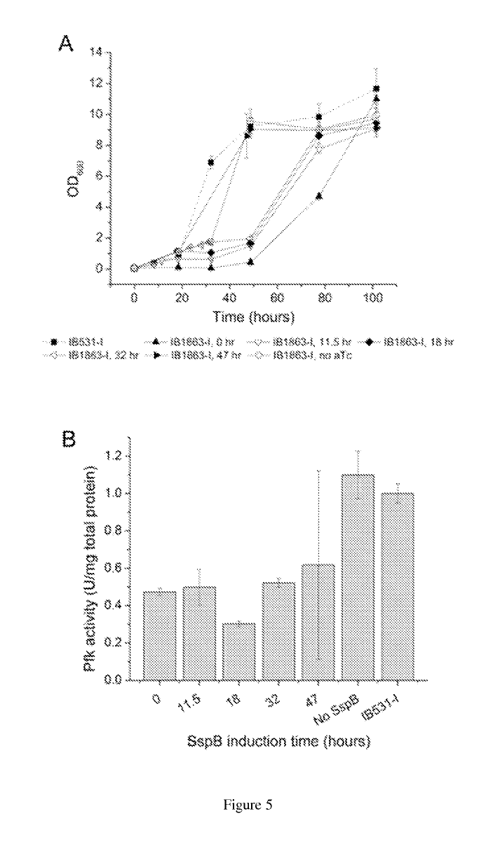

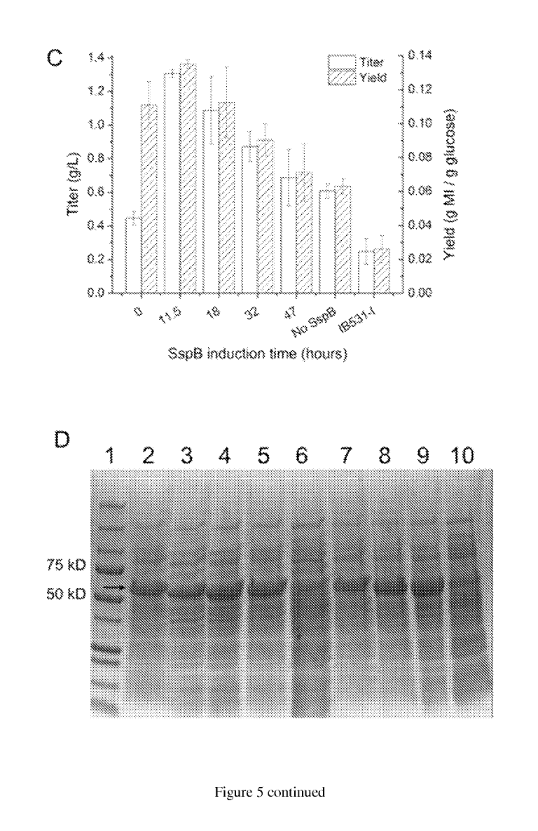

Strong growth arrest was observed after induction of SspB in IB1863-I, except in the case of aTc addition at 47 hours, where the strain was in stationary phase (FIG. 5A). Activity measurements at 48 hours indicated an average 56% reduction in Pfk activity across all samples with SspB induced relative to those without SspB induction for IB1863-I and a 52% reduction in Pfk activity relative to the control strain IB531-I (FIG. 5B). The Pfk-I knockdown was consistent across SspB induction times, although for cultures with SspB induced at 47 hours, Pfk-I knockdown did differ between replicates, which may have been due to the short time available for Pfk-I degradation and the difference in relative growth phase of the replicates at that point.

Both theoretical (Anesiadis et al., 2008; Gadkar et al., 2005) and experimental studies (Solomon et al., 2012b; Soma et al., 2014) have indicated that there should be an optimal time for knockdown of a growth-coupled or essential gene within a fixed batch time to maximize product titer and yield. The theoretical work uses dynamic flux balance (dFBA) simulations to show that very early knockdown will result in the highest yields, but potentially incomplete carbon utilization within the batch, while later knockdown will result in lower yields, as more carbon is used for biomass, but faster consumption of the available substrate given the higher concentration of biocatalyst. The earliest knockdown time that allows complete consumption of glucose should then give the highest titer in the batch. The experimental work to date shows the tradeoff can be more nuanced, as very early induction of gene knockdown, while in theory delivering highest yields, can result in unexpectedly poor growth (Soma et al., 2014). As expected, MI yield and titer varied as a function of induction time, with the highest yield and titers when SspB was induced at 11.5 hours (OD=0.5). This resulted in a more than two-fold improvement in titers due to controlled Pfk-I degradation, with 1.31 g/L MI produced with SspB induction at OD=0.5 compared to 0.61 g/L MI without SspB induction, and a five-fold improvement over the 0.25 g/L MI observed for the parent strain IB531-I (FIG. 5C). Delaying induction of SspB beyond 11.5 hours in this 78 hour batch resulted in the expected lower yields and lower titers, as more carbon was directed into biomass formation, with yields converging to those shown in the control without SspB induction. At the other extreme, induction of SspB at inoculation resulted in lower titers due to incomplete consumption of glucose after 78 hours. However, the yield was also lower, falling outside the trend of expected higher yields at earlier SspB induction times. SDS-PAGE analysis indicated that INO1 was expressed at levels comparable to the other conditions (FIG. 5D). Therefore, the reduced yield may have been due to broader limitations in metabolic capacity when early glucose uptake was restricted, rather than being due to poor recombinant protein expression. These results demonstrate that it is possible to improve myo-inositol yields and titers by delaying knockdown of Pfk-I rather than using a static knockdown strategy, and that the optimal induction time will depend on the desired batch length. Further optimization of the timing of both INO1 expression and Pfk-I knockdown could be used to minimize the time required for biomass formation and allow for the highest flux toward myo-inositol within the biomass formed, maximizing productivity rates.

By 78 hours, formation of biomass had resumed even in cultures in which growth had been arrested. This corresponded with some recovery in Pfk activity, likely indicating selection for cells that either exclude inducer or have accumulated mutations preventing the degradation of Pfk-I. While it would be most desirable to maintain full knockdown throughout the course of the fermentation, there is still a significant period of reduced activity, which provides sufficient time for flux redirection. Having a larger proportion of cells in the culture with increased Pfk activity may result in decreased myo-inositol yield due to increased glucose utilization for biomass, but the trend shows that earlier induction of Pfk degradation, leaving the most glucose available for any “escaped” cells to consume, still resulted in higher yields and titers. As a result, escape is likely not driving the differences in yield and titer.

Analysis of lysates by SDS-PAGE showed similar expression of INO1 during exponential growth at 18 hours for IB5314 and for IB18634 with and without SspB induction (FIG. 5D), indicating that differences in initial expression of INO1 were not a primary factor in influencing yield. At 48 hours, cultures containing IB5314 and untreated IB1863-I had reached stationary phase, and showed reduced INO1 expression, while those cultures in growth arrest after SspB induction showed high levels of INO1 expression. Growth arrest due to dynamic Pfk-I knockdown yields a metabolic state in which recombinant protein levels can still be maintained.

### Application of the Pfk-I Valve in a PTS-Strain Background

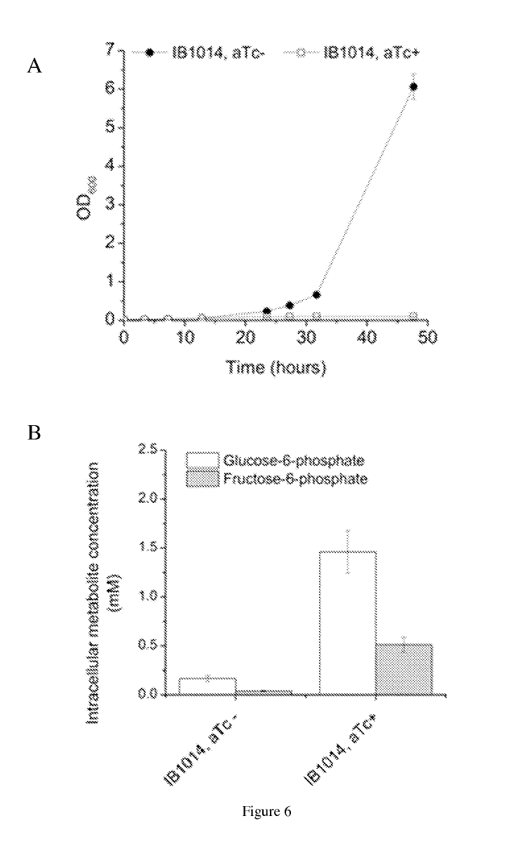

Maintaining a suitable glucose uptake rate can be a factor for maximizing MI productivity. However, uptake of glucose via the phosphotransferase system (PTS) may be limited by downstream availability of phosphoenolpyruvate (PEP) when flux through Pfk-I is restricted. A PTS-glucose+ strain was generated via deletion of ptsHIcrr and constitutive expression of galP (manipulations which were previously shown to impart a PTS-glucose+ phenotype; De Anda et al., 2006; Solomon et al., 2012a) to test the effect of Pfk-I knockdown when glucose uptake is independent of the PTS. This strain, IB1014, showed a significant reduction in growth on glucose compared to IB1863, with a baseline growth rate of 0.13 hr-1 in glucose minimal medium at 30° C., less than half of that observed for IB1863. However, the behavior of the strain upon induction of SspB was still consistent and addition of aTc resulted in growth arrest, with a reduction in growth rate of over 90% (FIG. 6A). G6P levels were also measured in the strain without and without induction of SspB. Similar to IB1863, induction of SspB resulted in an increase in the G6P pool in IB1014 (FIG. 6B). Pfk-I knockdown may be applicable for G6P accumulation in a PTS-background and the growth arrest observed in IB1863 was not dependent upon PEP limitation driving reduced glucose uptake.

Further, IB1014 was transformed with pTrc-INO1 to determine myo-inositol production. In the modified MOPS minimal medium with 10 g/L glucose used for testing of IB1863-I, IB1014-I showed no growth after 36 hours when INO1 was expressed at inoculation. IB1014-I was then tested in the same base medium and with the same INO1 induction conditions, but with additional supplementation of 0.2% casamino acids. Casamino acid supplementation restored growth, although IB1014-I was impaired in growth relative to the wild type (PTS+) control IB531-I. At various time points during the fermentation in casamino acid supplemented medium, 100 ng/ml aTc was added to cultures of IB1014-I knock down Pfk-I activity. The growth patterns were qualitatively similar to those seen for IB1863-I, with growth arrest occurring at initial aTc addition and eventual accumulation of biomass at long times. Culture supernatant was analyzed by HPLC, but MI titers were below 0.1 g/L in IB1014-I and could not be accurately quantified. However, the strain may be improved through adaptation on glucose or upregulation of Glk.

The flexibility afforded through dynamic control allowed E. coli strain IB1863-1 to be repurposed for use with different feedstocks. In addition to using glucose as a feedstock, myo-inositol can be produced from at least xylose and arabinose, which are metabolized to F6P and glyceraldehyde-3 phosphate. By blocking F6P consumption through Pfk-I knockdown, this system can also be used to flexibly produce MI from those feedstocks. Induction time can be used to control the feedstock used for biomass accumulation versus for MI production. In a mixture of 10 g/L glucose and 6 g/L xylose, inducing SspB expression at OD=7.9 (glucose depletion) resulted in a 26% improvement in yields and titers compared to the uninduced culture at 96 hours, indicating redirection of xylose fluxes into MI production (FIG. 6).

### Discussion

In the production of MI, there is a direct competition between cellular growth and carbon flux toward product. Given this tradeoff, it is very difficult to engineer a static knockdown strategy which will not result in detrimental effects to the host strain, including poor growth and poor expression of recombinant proteins. To overcome this, development was focused on a host strain that retained the positive characteristics of the parent strain with regard to growth and glucose uptake, but could be switched to a production mode with reduced carbon utilization for biomass.

E. coli strain IB1863 was engineered to make Pfk-I the control point for utilizing G6P and F6P, thereby linking cellular growth to steady-state levels of the Pfk-I enzyme. Through addition of an SspB-dependent degradation tag to the coding sequence of Pfk-I, the steady-state level could be controlled by expression of SspB from an inducible promoter. After these changes, IB1863 showed a modest reduction in growth rate compared to the parent strain in untreated cultures, but addition of aTc could be used to control glucose uptake, reducing growth by more than 80%. Further, coupling Pfk-I control with expression of INO1 permitted a two-fold increase in MI yield and titer when utilizing glucose as a sole carbon source.

Notably, the greatest improvement in yield and titer came with delayed induction of SspB, indicating that the dynamic control of Pfk-I, rather than static downregulation, was useful for achieving increased yields. The full extent of the system can be further optimized, for example, by including combinations of different induction times and induction levels for both INO1 and SspB, thereby allowing a variety of biomass and production tradeoffs to be tested. One possibility for use of this system may include a fed-batch fermentation, where Pfk knockdown is induced after a suitable period of biomass formation and glucose is then fed at a rate matching the uptake needed for myo-inositol production and for minor glycolytic flux supporting cell maintenance.

Given that the deletion of pfkA in the Δzwf ΔpfkB background used is lethal, the productivity of a static knockout strain is zero, and dynamic control may be useful for this type of system. While the direct trade-off between yield and titer predicted in the dFBA work at the batch length tested in this study was not observed, as both titer and yield increased with dynamic control in this case, productivity trends may still be considered (e.g., for a fed-batch case relying on formation of a fixed initial amount of biomass).

In addition to testing of the system in a strain relying on glucose uptake via the PTS, the applicability of Pfk-I control in a strain where glucose uptake is independent of PEP utilization was also tested. Increases in the G6P pool could still be achieved in a PTS-background. While the particular PTS-strain, described herein, may require optimization to support MI production, ultimately, a strain where glucose uptake is decoupled from PEP utilization could offer higher yields for products derived from G6P. Reducing equivalents or ATP produced within a heterologous pathway could also be used to drive glucose uptake, maintaining glucose utilization even as metabolites in lower glycolysis are depleted.

Controlled degradation of Pfk-I as a novel method for redirecting flux of G6P into a heterologous pathway has been described. Upon addition of aTc to induce expression of SspB, Pfk-I was degraded and carbon flux into biomass was halted. This system was used to achieve improvements in both yield and titer of MI on glucose as a sole carbon source. The rapid, dynamic nature of the switching allowed desirable cellular phenotypes (rapid growth, high expression of recombinant proteins) to be preserved during the growth phase of a fermentation, while still achieving reduced flux into central metabolism during the production phase. While this system was designed to be coupled with the first step in a pathway for glucaric acid production, it could be more broadly applied to any pathway with G6P or F6P as a branch point. This could include production of other myo-inositol derivatives, including scyllo-inositol, which has been studied for therapeutic uses (Yamaoka et al., 2011) or redirection of flux into pathways into the pentose phosphate pathway in response to cellular demand for NADPH, which is important when expressing pathways with high cofactor requirements, such as fatty acid biosynthesis.

### Materials and Methods of Example 1

### Strains and Plasmids

E. coli strains and plasmids used in this study are listed in Table 1. E. coli strain DH10B was used for molecular cloning and plasmid preparation. Production strains were constructed utilizing MG1655 A endA (IB531) as a parent strain. Knockouts of zwf, pfkB, and sspB were accomplished via sequential P1 transduction from Keio collection donor strains (Baba et al., 2006). The kanamycin resistance cassette was removed after each transduction via expression of FLP recombinase from pCP20 (Datsenko and Wanner, 2000). The native pfkA locus was replaced with a version containing a constitutive promoter (apFAB114) and 5′ UTR from the BIOFAB library (Mutalik et al., 2013) and the degradation tag AADENYSENYADAS (SEQ ID NO: 3) (McGinness et al., 2006). The replacement at the pfkA locus was carried out via a “landing pad” method (Kuhlman and Cox, 2010). The pfkA coding sequence was amplified from the E. coli genome with primers which appended the promoter and UTR at the 5′ end and the degradation tag at the 3′ end of the gene. This product was cloned into the vector pTKIP-neo by restriction digest with HindIII and KpnI, yielding pTKIP-114pfkA(DAS+4). Lambda-red mediated recombination was used to introduce the tetracycline resistance marker and “landing pad” sequences amplified from pTKS/CS into the genome at the pfkA locus. The resultant strain was then transformed with pTKRED and pTKIP-114pfkA(DAS+4), and integration of the construct from the pTKIP plasmid into the genome was achieved as described previously (Kuhlman and Cox, 2010). The kanamycin resistance cassette remaining after integration was cured by expression of FLP recombinase from pCP20 to yield strain IB1643.

Integration of the tetR-P_{LteOt}-sspB cassette into the genome was carried out via “clonetegration” (St-Pierre et al., 2013). The coding sequence of sspB was amplified from the E. coli genome and cloned into pKVS45 via restriction digest to yield pKVS-SspB. The vector pKVS45 includes a TetR expression cassette originally amplified from pWW308 (Solomon et al., 2012b). The entire tetR-P_{LtetO}-sspB cassette was amplified from pKVS-SspB. The pOSIP-CH backbone was also PCR amplified and cycled 10× with the tetR-P_{LteOt}-sspB fragment according to the protocol for circular polymerase extension cloning (CPEC) (Quan and Tian, 2009). The CPEC product was used to transform strain IB1643 for integration at the HK022 locus. The phage integration genes and antibiotic resistance cassette were cured with pE-FLP as described in the previously published protocol (St-Pierre et al., 2013) to yield strain IB1863.

For construction of IB1014, integration cassettes for deletion of ptsHIcrr and replacement of the native galP promoter with a strong constitutive promoter were PCR amplified from the genome of previously developed phosphotransferase system deficient (PTS-), glucose utilizing (glucose+) strains (Solomon et al., 2012a). These cassettes contained the desired genomic deletion or promoter replacement, a kanamycin resistance cassette, and the upstream and downstream genomic homology. The PCR cassettes were sequentially integrated into IB1863 via lambda-red mediated recombination using the helper plasmid pKD46, and each kanamycin resistance cassette was cured by expression of FLP recombinase from pCP20 (Datsenko and Wanner, 2000).

During all integration steps, colonies were screened via colony PCR with OneTaq master mix. PCR amplifications for cloning or genomic integration were carried out with Q5 polymerase. Enzymes utilized for PCR amplification, restriction digests, and ligation were obtained from New England Biolabs (Ipswich, Mass.). Oligonucleotides were obtained from Sigma-Genosys (St. Louis, Mo.).

### Culture Medium and Conditions

For plasmid preparation and genetic manipulations, strains were cultured in Luria-Bertani (LB) medium at either 30° or 37° C. Temperature sensitive plasmids were cured at 42° C.

M9 minimal medium supplemented with either 0.4% glucose or 0.4% glycerol was utilized for initial screening of promoters for pfkA. All additional cultures for measurement of growth and production were carried out at 30° C. in a modified MOPS medium containing 10 g/L D-glucose, 3 g/L NH₄Cl, 1 g/L K₂HPO₄, 2 mM MgSO₄, 0.1 mM CaCl₂, 40 mM MOPS, 4 mM tricine, 50 mM NaCl, 100 mM Bis-Tris, 134 μM EDTA, 31 μM FeCl₃, 6.2 μM ZnCl₃, 0.76 μM CuCl₂, 0.42 μM CoCl₂, 1.62 μM H₃BO₃, and 0.081 μM MnCl₂. When noted, the medium was supplemented with an additional 0.2% casamino acids. For strains containing pTrc-INO1, carbenicillin (100 μg/mL) was added for plasmid maintenance. Seed cultures were initiated using a 1:100-1:500 dilution from LB cultures and were grown at 30° C. for 18-24 hours in modified MOPS, until mid-exponential phase was reached. Working cultures of 50 ml in 250 ml baffled shake flasks were inoculated to OD=0.05 from seed cultures, and for myo-inositol production experiments, 50 μM β-D-1-thiogalactopyranoside (IPTG) was added at inoculation. For induction of SspB in strain IB1863, anhydrotetracycline (aTc) was added at the times and concentrations indicated in the results section. Samples were taken periodically for measurement of enzyme activity, protein levels, and extracellular metabolites.

### Phosphofructokinase Activity Assays and Western Blotting

All enzymatic activity assays were carried out on crude lysates. For preparation of lysates, samples of 5-10 ml of cell culture were collected, frozen at −80° C., and then resuspended in 50 mM Tris-HCl, pH 7.4 (0.25-1 ml, depending on cell density). Cells were lysed via bead beating for 5 minutes and lysates clarified by centrifugation at 15,000×g for 15 minutes. Phosphofructokinase activity was assayed using a protocol adapted from Kotlarz and Buc (Kotlarz and Buc, 1982; Kotlarz et al., 1975). The assay mixture consisted of 0.1 M Tris-HCl (pH 8.2), 10 mM MgCl2, 1 mM ATP, 0.2 mM β-NADH, 1 mM fructose-6-phosphate (F6P), 1 mM NH4Cl, 0.01% Triton X-100, 0.83 U aldolase, 0.42 U triosephosphate isomerase, and 0.42 U glycerophosphate dehydrogenase. Reaction progress was followed by measurement of absorbance at 340 nm. One unit of Pfk activity was defined as the amount required to convert 1.0 μmole of ATP and D-fructose 6-phosphate to ADP and fructose 1,6-bisphosphate per minute at pH 8.2 and room temperature.

For Western blots to confirm disappearance of Pfk-I, lysis was carried out as for enzymatic assays. A 4-20% SDS-PAGE gel was run with 10 μg total protein per lane. Proteins were transferred from the PAGE gel to a nitrocellulose membrane and excess binding sites were blocked using 5% dry milk in TBS. The membrane was incubated at 4° C. overnight with anti-Pfk-I rabbit polyclonal antibody (6.7 μg/ml) custom prepared by Genscript (Piscataway Township, N.J.), rinsed, and incubated with HRP-conjugated goat anti-rabbit IgG polyclonal antibody (Santa Cruz Biotechnology, Dallas, Tex.) and StrepTactin-HRP (Bio-rad, Hercules, Calif.) at room temperature for one hour. Bands were visualized by treatment with Western blotting Luminol Reagent (Santa Cruz Biotechnology).

Total protein levels for normalization of enzymatic activities and Western blot loadings were measured using a modified Bradford assay (Zor and Selinger, 1996).

### Measurement of Intracellular Glucose-6-Phosphate and Fructose-6-Phosphate

Cultures of IB531, IB1863, and IB1014 were grown in triplicate in modified MOPS medium. Working cultures were inoculated to OD=0.02 and incubated at 30° C. For measurement of G6P buildup after Pfk-I degradation, aTc was added to the culture one hour before collection of the cell mass.

For intracellular metabolite measurements, 20-30 ml of cells in exponential phase (OD=0.3-0.9) were collected via vacuum filtration through 0.45 μM Metricel GN-6 filters (Pall, Port Washington, N.Y.). After filtration of the culture, the filters with cell mass were immediately immersed in 10 ml of 75% ethanol solution at 80° C. The solution was vortexed for 30 seconds, incubated at 80° C. for 3 minutes, and vortexed again. The filter paper was then removed and the solution centrifuged for 10 minutes at 5000×g to remove cell debris. Incubation in boiling 75% ethanol for 3 minutes has been previously shown to result in good extraction of sugar phosphates (Gonzalez et al., 1997). The solutions were stored at −80° C. until evaporation for analysis.

The 75% ethanol solution was completely evaporated and the remaining solids resuspended in 200 μL water. Any solids which could not be dissolved in water were pelleted by centrifugation at 15,000×g for 5 minutes. Enzymatic assays for G6P and F6P were carried out in 96-well plates in a Tecan infinite F200Pro plate reader (Männedorf, Switzerland). The assay solution consisted of 0.2 M triethanolamine, 0.2 mM NADP, and 5 mM MgCl₂ (Bergmeyer et al., 1983). After stabilization of the baseline, glucose-6-phosphate dehydrogenase was added to the assay solution and G6P levels were analyzed by following NADPH generation via fluorescence (excitation 340 nm, emission 450 nm). After complete turnover of G6P, F6P levels were analyzed by the addition phosphoglucose isomerase, which converted the remaining F6P to G6P. Intracellular metabolite levels were estimated assuming 0.4 gDCW/OD unit (Tseng et al., 2009) and an intracellular volume of 2 ml/gDCW, which would be expected for E. coli at a similar growth rate on glucose (Hiller et al., 2007).

### Measurement of Extracellular Metabolites

Glucose, acetate, and myo-inositol levels were quantified by high performance liquid chromatography (HPLC) on an Agilent 1100 or 1200 series instrument (Santa Clara, Calif.) with an Aminex HPX-87H anion exchange column (300 mm by 7.8 mm; Bio-Rad Laboratories, Hercules, Calif.). Sulfuric acid (5 mM) at a flow rate of 0.6 mL/min was used as the mobile phase. Compounds were quantified from 10 μL sample injections using refractive index and diode array detectors. Column and refractive index detector temperatures were held at 35° C. Glucose uptake and acetate production rates were calculated using an estimated cell mass of 0.4 gDCW/OD unit (Tseng et al., 2009).

### Tables of Example 1

TABLE 1
Strains and plasmids

| Strain/plasmid | Genotype | Reference/source |
|---|---|---|
| Strains | | |
| DH10B | F⁻ mcrA Δ(mrr-hsdRMS-mcrBC) φ80lacZΔM15 | Life Technologies (Carlsbad, CA) |
| | ΔlacX74 recA1 endA1 araD139 Δ(ara, leu)7697 | |
| | galU galK λ-rpsL nupG | |
| MG1655 | F⁻ λ⁻ ilvG⁻ frb-50 rph-1 | ATCC #700926 |
| IB531 | MG1655 ΔendA | Prather Lab |
| IB1375 | MG1655 ΔendA Δzwf | This study |
| IB1379 | MG1655 ΔendA Δzwf ΔpfkB | This study |
| IB1489 | MG1655 ΔendA Δzwf ΔpfkB ΔsspB | This study |
| IB1643 | MG1655 ΔendA Δzwf ΔpfkB ΔsspB pfkA::114- | This study |
| | pfkA(DAS + 4) | |
| IB1863 | MG1655 ΔendA Δzwf ΔpfkB ΔsspB pfkA::114- | This study |
| | pfkA(DAS + 4) HK022::tetR-Ptet-sspB | |
| IB1014 | MG1655 ΔendA Δzwf ΔpfkB ΔsspB ΔptsHIcrr | This study |
| | pfkA::114-pfkA(DAS + 4) HK022::tetR-Ptet-sspB | |
| | galP^{q} | |
| IB531-I | IB531/pTrc-INO1 | This study |
| IB1863-I | IB1863/pTrc-INO1 | This study |
| IB1014-I | IB1014/pTrc-INO1 | This study |
| JW3197-1 | F−, Δ(araD-araB)567, ΔlacZ4787(::rrnB-3), λ⁻, | (Baba et al., 2006) |
| | ΔsspB756::kan, rph-1, Δ(rhaD-rhaB)568, hsdR514 | |
| JW5280-1 | F−, Δ(araD-araB)567, ΔlacZ4787(::rrnB-3), λ⁻, | (Baba et al., 2006) |
| | ΔpfkB722::kan, rph-1, Δ(rhaD-rhaB)568, hsdR514 | |
| JW1841-1 | F−, Δ(araD-araB)567, ΔlacZ4787(::rrnB-3), λ⁻, | (Baba et al., 2006) |
| | Δzwf-777::kan, rph-1, Δ(rhaD-rhaB)568, hsdR514 | |
| Plasmids | | |
| pCP20 | Rep^{a}, Amp^{R}, Cm^{R}, FLP recombinase expressed by λ | CGSC #7629 |
| | pᵣ under control of λ cI857 | |
| pKD46 | oriR101, repA101ts, Amp^{R}, araC, araBp-λγ-λβ- | CGSC #7739 |
| | λexo | |
| pE-FLP | oriR101, repA101ts, Amp^{R}, FLP recombinase | (St-Pierre et al., 2013) |
| | expressed by pE | |
| pKVS45 | p15A, Amp^{R}, tetR, P_{Tet} | (Solomon et al., 2012b) |
| pKVS-SspB | pKVS45 with RBS B0034 + E. coli SspB inserted at | This study |
| | the EcoRI and BamHI sites | |
| pOSIP-CH | pUC ori, RK6γ ori, Cm^{R}, attP HK022, ccdB, | (St-Pierre et al., 2013) |
| | HK022 integrase expressed by λ pᵣ under control of | |
| | λ cI857 | |
| pTKIP-neo | ColE1(pBR322) ori, Amp^{R}, Kan^{R} | (Kuhlman and Cox, 2010) |
| pTKRED | oriR101, repA101ts, Spec^{R}, araC, P_{lac} λ_{γ} λ_{β} λₑₓₒ lacI, | (Kuhlman and Cox, 2010) |
| | P_{araB} I-SceI | |
| pTKS/CS | p15A, Cm^{R}, P_{lacIq} tetA | (Kuhlman and Cox, 2010) |
| pTrc-INO1 | pTrc99A with S. cerevisiae INO1 inserted at the | (Moon et al., 2009) |
| | EcoRI and HindIII sites | |

### REFERENCES FOR EXAMPLE 1

Andersen, J. B., Sternberg, C., Poulsen, L. K., Bjorn, S. P., Givskov, M., Molin, S., 1998. New unstable variants of green fluorescent protein for studies of transient gene expression in bacteria. Appl Environ Microbiol. 64, 2240-6.
Anesiadis, N., Cluett, W. R., Mahadevan, R., 2008. Dynamic metabolic engineering for increasing bioprocess productivity. Metab Eng. 10, 255-66.
Anesiadis, N., Kobayashi, H., Cluett, W. R., Mahadevan, R., 2013. Analysis and design of a genetic circuit for dynamic metabolic engineering. ACS Synthetic Biology.
Baba, T., Ara, T., Hasegawa, M., Takai, Y., Okumura, Y., Baba, M., Datsenko, K. A., Tomita, M., Wanner, B. L., Mori, H., 2006. Construction of Escherichia coli K-12 in-frame, single-gene knockout mutants: the Keio collection. Mol Syst Biol. 2.
Bar-Even, A., Noor, E., Savir, Y., Liebermeister, W., Davidi, D., Tawfik, D. S., Milo, R., 2011. The Moderately Efficient Enzyme: Evolutionary and Physicochemical Trends Shaping Enzyme Parameters. Biochemistry. 50, 4402-4410.
Bennett, B. D., Kimball, E. H., Gao, M., Osterhout, R., Van Dien, S. J., Rabinowitz, J. D., 2009. Absolute metabolite concentrations and implied enzyme active site occupancy in Escherichia coli. Nat Chem Biol. 5, 593-9.
Bergmeyer, H. U., Bergmeyer, J., Grassl, M., 1983. Methods of enzymatic analysis/editor-in-chief, Hans Ulrich Bergmeyer; editors, Jurgen Bergmeyer and Marianne Grassl. Weinheim; Deerfield Beach, Fla.: Verlag Chemie, c1983-3rd ed.
Callura, J. M., Cantor, C. R., Collins, J. J., 2012. Genetic switchboard for synthetic biology applications. Proceedings of the National Academy of Sciences of the United States of America. 109, 5850-5.
Cardinale, S., Arkin, A. P., 2012. Contextualizing context for synthetic biology-identifying causes of failure of synthetic biological systems. Biotechnology Journal. 7, 856-866.
Datsenko, K. A., Wanner, B. L., 2000. One-step inactivation of chromosomal genes in Escherichia coli K-12 using PCR products. Proceedings of the National Academy of Sciences of the United States of America. 97, 6640-5.
Davis, J. H., Baker, T. A., Sauer, R. T., 2011. Small-Molecule Control of Protein Degradation Using Split Adaptors. ACS Chemical Biology.
De Anda, R., Lara, A. R., Hernandez, V., Hernandez-Montalvo, V., Gosset, G., Bolivar, F., Ramirez, 0. T., 2006. Replacement of the glucose phosphotransferase transport system by galactose permease reduces acetate accumulation and improves process performance of Escherichia coli for recombinant protein production without impairment of growth rate. Metabolic Engineering. 8, 281-290.
Delgado, J., Meruane, J., Liao, J. C., 1993. Experimental determination of flux control distribution in biochemical systems: in vitro model to analyze transient metabolite concentrations. Biotechnol Bioeng. 41, 1121-8.
Farmer, W. R., Liao, J. C., 2000. Improving lycopene production in Escherichia coli by engineering metabolic control. Nat Biotechnol. 18, 533-7.
Gadkar, K. G., Doyle Iii, F. J., Edwards, J. S., Mahadevan, R., 2005. Estimating optimal profiles of genetic alterations using constraint-based models. Biotechnology and Bioengineering. 89, 243-251.
Gonzalez, B., Francois, J., Renaud, M., 1997. A rapid and reliable method for metabolite extraction in yeast using boiling buffered ethanol. Yeast. 13, 1347-1355.
Hansen, C. A., Dean, A. B., Draths, K. M., Frost, J. W., 1999. Synthesis of 1,2,3,4-Tetrahydroxybenzene from d-Glucose:K %0 Exploiting myo-Inositol as a Precursor to Aromatic Chemicals. Journal of the American Chemical Society. 121, 3799-3800.
Hiller, J., Franco-Lara, E., Weuster-Botz, D., 2007. Metabolic profiling of Escherichia coli cultivations: evaluation of extraction and metabolite analysis procedures. Biotechnol Lett. 29, 1169-1178.
Holtz, W. J., Keasling, J. D., 2010. Engineering Static and Dynamic Control of Synthetic Pathways. Cell. 140, 19-23.
Kamionka, A., Bogdanska-Urbaniak, J., Scholz, O., Hillen, W., 2004. Two mutations in the tetracycline repressor change the inducer anhydrotetracycline to a corepressor. Nucleic Acids Research. 32, 842-847.
Keseler, I. M., Collado-Vides, J., Santos-Zavaleta, A., Peralta-Gil, M., Gama-Castro, S., Muiliz-Rascado, L., Bonavides-Martinez, C., Paley, S., Krummenacker, M., Altman, T., Kaipa, P., Spaulding, A., Pacheco, J., Latendresse, M., Fulcher, C., Sarker, M., Shearer, A. G., Mackie, A., Paulsen, I., Gunsalus, R. P., Karp, P. D., 2011. EcoCyc: a comprehensive database of Escherichia coli biology. Nucleic Acids Research. 39, D583-D590.
Kotlarz, D., Buc, H., 1982. [11] Phosphofructokinases from Escherichia coli. In: Willis, A. W., (Ed.), Methods in Enzymology. vol. Volume 90. Academic Press, pp. 60-70.
Kotlarz, D., Garreau, H., Buc, H., 1975. Regulation of the amount and of the activity of phosphofructokinases and pyruvate kinases in Escherichia coli. Biochimica et Biophysica Acta (BBA)—General Subjects. 381, 257-268.
Kuhlman, T. E., Cox, E. C., 2010. Site-specific chromosomal integration of large synthetic constructs. Nucleic Acids Research.
Lee, J. W., Na, D., Park, J. M., Lee, J., Choi, S., Lee, S. Y., 2012. Systems metabolic engineering of microorganisms for natural and non-natural chemicals. Nat Chem Biol. 8, 536-546.
Lutz, R., Bujard, H., 1997. Independent and tight regulation of transcriptional units in Escherichia coli via the LacR/O, the TetR/O and AraC/I1-I2 regulatory elements. Nucleic Acids Res. 25, 1203-10.
Majumder, A. L., Johnson, M. D., Henry, S. A., 1997. 1-myo-Inositol-1-phosphate synthase. Biochimica et Biophysica Acta (BBA)—Lipids and Lipid Metabolism. 1348, 245-256.
McGinness, K. E., Baker, T. A., Sauer, R. T., 2006. Engineering controllable protein degradation. Mol Cell. 22, 701-7.
Moon, T. S., Yoon, S. H., Lanza, A. M., Roy-Mayhew, J. D., Prather, K. L., 2009. Production of glucaric acid from a synthetic pathway in recombinant Escherichia coli. Appl Environ Microbiol. 75, 589-95.
Mutalik, V. K., Guimaraes, J. C., Cambray, G., Lam, C., Christoffersen, M. J., Mai, Q.-A., Tran, A. B., Paull, M., Keasling, J. D., Arkin, A. P., Endy, D., 2013. Precise and reliable gene expression via standard transcription and translation initiation elements. Nat Meth. 10, 354-360.
Quan, J., Tian, J., 2009. Circular Polymerase Extension Cloning of Complex Gene Libraries and Pathways. PLoS ONE. 4, e6441.
Shiue, E. C., Brockman, I. M., Prather, K. L. J., submitted. Improving Product Yields on D-Glucose in Escherichia coli via Alternative Carbon Sources. Biotechnol Bioeng.
Solomon, K. V., Moon, T. S., Ma, B., Sanders, T. M., Prather, K. L. J., 2012a. Tuning Primary Metabolism for Heterologous Pathway Productivity. ACS Synthetic Biology.
Solomon, K. V., Sanders, T. M., Prather, K. L., 2012b. A dynamic metabolite valve for the control of central carbon metabolism. Metab Eng. 14, 661-71.
Soma, Y., Tsuruno, K., Wada, M., Yokota, A., Hanai, T., 2014. Metabolic flux redirection from a central metabolic pathway toward a synthetic pathway using a metabolic toggle switch. Metab Eng. 23, 175-84.
St-Pierre, F., Cui, L., Priest, D. G., Endy, D., Dodd, I. B., Shearwin, K. E., 2013. One-Step Cloning and Chromosomal Integration of DNA. ACS Synthetic Biology.
Stephanopoulos, G., Aristidou, A. A., Nielsen, J. H., 1998. Metabolic engineering: principles and methodologies. Academic Press, San Diego.
Torella, J. P., Ford, T. J., Kim, S. N., Chen, A. M., Way, J. C., Silver, P. A., 2013. Tailored fatty acid synthesis via dynamic control of fatty acid elongation. Proceedings of the National Academy of Sciences. 110, 11290-11295.
Tseng, H.-C., Martin, C. H., Nielsen, D. R., Prather, K. L. J., 2009. Metabolic Engineering of Escherichia coli for Enhanced Production of (R)- and (S)-3-Hydroxybutyrate. Applied and Environmental Microbiology. 75, 3137-3145.
Tyo, K. E. J., Kocharin, K., Nielsen, J., 2010. Toward design-based engineering of industrial microbes. Current Opinion in Microbiology. 13, 255-262.
Werpy, T., Petersen, G., Top value added chemical from biomass, volume 1—results of screening for potential candidates from sugars and synthetic gas. U.S. Department of Energy, Washington, D.C., 2004.
Woolston, B. M., Edgar, S., Stephanopoulos, G., 2013. Metabolic Engineering: Past and Future. Annual Review of Chemical and Biomolecular Engineering. 4, 259-288.
Yamaoka, M., Osawa, S., Morinaga, T., Takenaka, S., Yoshida, K.-i., 2011. A cell factory of Bacillus subtilis engineered for the simple bioconversion of myo-inositol to scyllo-inositol, a potential therapeutic agent for Alzheimer's disease. Microbial Cell Factories. 10, 1-6.
Zor, T., Selinger, Z., 1996. Linearization of the Bradford Protein Assay Increases Its Sensitivity: Theoretical and Experimental Studies. Analytical Biochemistry. 236, 302-308.

### Example 2: Improvement of Glucaric Acid Production in E. coli Via Dynamic Control of Metabolic Fluxes

### Abstract

D-glucaric acid can be used as a building block for biopolymers as well as in the formulation of detergents and corrosion inhibitors. A biosynthetic route for production in E. coli has been developed (Moon et al., 2009), but previous work with the glucaric acid pathway has indicated that competition with endogenous metabolism may limit carbon flux into the pathway. Accordingly, provided herein is an E. coli strain where phosphofructokinase (Pfk) activity can be dynamically controlled and used for improving yields and titers of the glucaric acid precursor myo-inositol on glucose minimal medium. Further, applicability of this strain for glucaric acid production in a supplemented medium more relevant for scale-up studies, both under batch conditions and with glucose feeding via in situ enzymatic starch hydrolysis was determined. Glucaric acid titers could be improved by up to 42% with appropriately timed knockdown of Pfk activity during glucose feeding.

### Introduction

D-glucaric acid was identified by the United State Department of Energy as a top value-added chemical for production from biomass (Werpy and Petersen, 2004). It has a number of potential applications including use in biopolymers (Kiely and Chen, 1994) and as a detergent builder and corrosion inhibitor (Smith et al., 2012). Glucaric acid can be produced through nitric acid oxidation of glucose (Mehltretter and Rist, 1953) but a biological route to glucaric acid production could potentially provide several advantages, including mild processing conditions and high selectivity for the product of interest.

Production of D-glucaric acid in Escherichia coli was previously demonstrated via expression of heterologous enzymes from three different organisms (Moon et al., 2009). Titers of 1.13 g/L glucaric acid were achieved in strain BL21(DE3) in LB medium supplemented with 10 g/L glucose. Following demonstration of the initial pathway, some increases in glucaric acid titers were achieved through improved strategies for expression of the myo-inositol oxygenase (MIOX) enzyme, one of the limiting factors in glucaric acid production in LB supplemented with glucose or myo-inositol (Moon et al., 2010; Shiue and Prather, 2014). However, competition for glucose-6-phosphate (G6P) between native E. coli enzymes (phosphoglucosisomerase and glucose-6-phosphate dehydrogenase) and the first enzyme in the glucaric acid pathway, myo-inositol-1-phosphate synthase (INO1), is also a concern. High level expression of INO1 is required for detectable myo-inositol and glucaric acid production, indicating that it competes poorly with endogenous metabolism for substrate (Moon et al., 2009). Additionally, the second pathway enzyme, MIOX, appears to be stabilized by its substrate, myo-inositol, so more rapid accumulation of myo-inositol may help reduce limitations in MIOX activity as well (Moon et al., 2010).

Strategies for development of strains capable of accumulating G6P and directing greater fluxes of this metabolite into production of glucaric acid and myo-inositol are contemplated herein. By eliminating the pathways for glucose catabolism in the production strain, and feeding alternative carbon sources, high yields of glucaric acid from glucose could be achieved (Shiue et al., 2015). However, the rate of glucose uptake in this strain was quite slow, especially in minimal medium, and its use was limited to mixed sugar substrates.

While gene knockouts provide a static solution for redirecting fluxes in the cell (Kogure et al., 2007; Shiue et al., 2015), under many conditions, it may be advantageous to develop cells where dynamic changes in enzyme levels can be used to switch between substrate consumption for biomass formation and substrate conversion into product. Dynamic control of key enzymes can be used to facilitate more rapid initial accumulation of biomass, overcoming potential reductions in growth rate, and can eliminate the need for supplementation of the medium or addition of secondary carbon sources required with some gene knockouts (Anesiadis et al., 2008; Gadkar et al., 2005). At the desired time, activity of the target enzyme(s) can be reduced through decreasing transcription (Scalcinati et al., 2012; Solomon et al., 2012; Soma et al., 2014) or translation (Williams et al., 2015) of the enzyme, or initiating rapid degradation (Brockman and Prather, 2015; Torella et al., 2013). Dynamically controlling the phosphofructokinase (Pfk) activity in the cell via controlled degradation, the pools of G6P could be increased during growth on glucose minimal medium, along with the yields and titers of the glucaric acid precursor myo-inositol (Brockman and Prather, 2015). Provided herein is a system for production of glucaric acid from glucose in a semi-defined medium under batch conditions and a fed-batch condition simulated by glucose release from in situ enzymatic starch hydrolysis. Improvements in glucaric acid titer of up to 42% were achieved through appropriately timed induction of Pfk activity knockdown during the fermentation.

### Results

Glucaric acid production was screened in strain IB1486-GA. This strain was derived from a previously developed strain, IB1863, where Pfk activity can be dynamically controlled through addition of aTc (Brockman and Prather, 2015). A modified SsrA tag was added to the coding sequence of pfkA in this strain, which results in slow degradation of the phosphofructokinase-I (Pfk-I) protein in the absence of the adapter protein SspB, but more rapid degradation of Pfk-I in the presence of SspB (McGinness et al., 2006). In IB1863, aTc addition induced SspB expression, resulting in rapid depletion of Pfk-I and buildup of intracellular G6P in glucose minimal medium. IB1486 comprises the same modifications, along with additional knockouts of gudD and uxaC, thereby preventing glucarate dehydratase and urinate isomerase activity respectively, to prevent glucaric acid catabolism and the DE3 lysogen for expression of T7 RNA polymerase. In the IB1863 strain, dynamic knockdown of Pfk activity could result in increased production of the glucaric acid precursor myo-inositol in glucose minimal medium, but that correct timing of aTc addition was required to achieve maximum yields and titers. Early switching to “production mode” by aTc addition resulted in insufficient time for protein expression and formation of biomass to serve as biocatalyst. However, late switching resulted in more utilization of glucose for growth, and less remaining glucose to be redirected into product formation.

To achieve increased glucaric acid production, the optimal aTc addition time was determined. Differences in the optimal aTc addition time for this strain may be due to changes in cellular growth rate from the burden of expressing the complete glucaric pathway and to medium composition changes relative to the glucose minimal medium previously performed. The modified MOPS glucose minimal medium previously used for myo-inositol production (Brockman and Prather, 2015) was initially used for glucaric acid production; however, lag times of approximately 48 hours were observed, which may have been due to the burden associated with expression of all three pathway proteins. In addition to glucose, the T12 medium used for testing glucaric acid production also contained yeast extract and soytone, which provided supplemental carbon sources. While glucose could primarily be used as a feedstock for glucaric acid production, additional carbon supplementation in the medium reduced batch time and simulated a potential semi-defined, scale-up medium.

To rapidly test a variety of aTc addition times in triplicate, cultures were grown in 48-well flower plates in a BioLector microbioreactor system. Glucaric acid production was screened in T12 medium in batch conditions (15 g/L glucose at inoculation) and in simulated fed batch conditions, where 3-5 g/L glucose was added at inoculation, and additional glucose was released slowly from 10-12 g/L starch by the addition of amyloglucosidase.

### Screening Batch Conditions for Timing of Pfk Knockdown

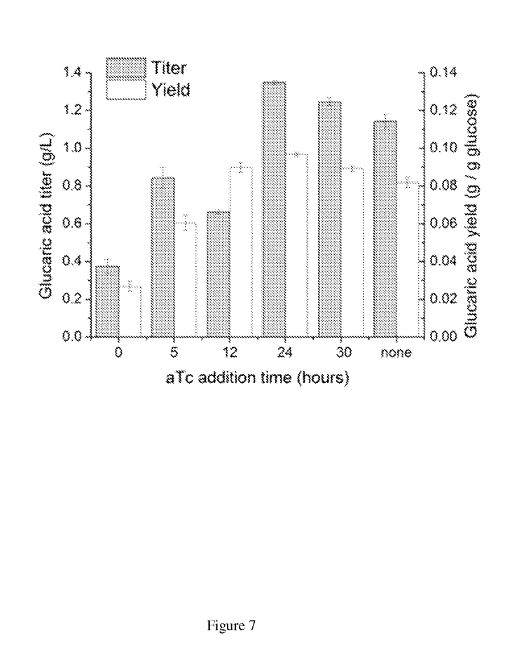

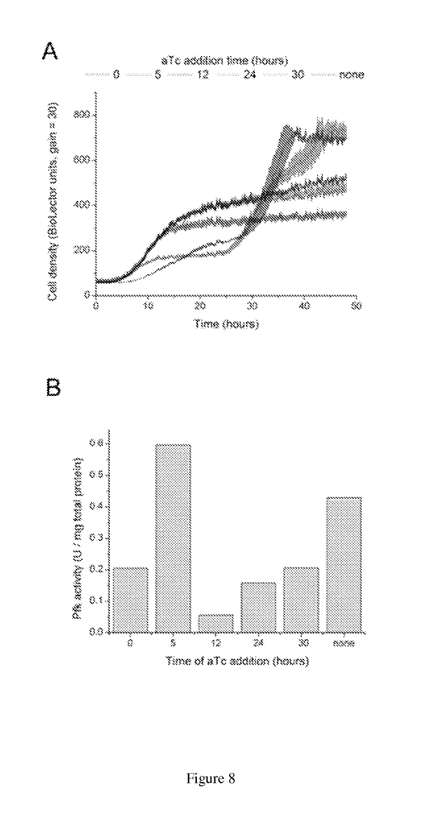

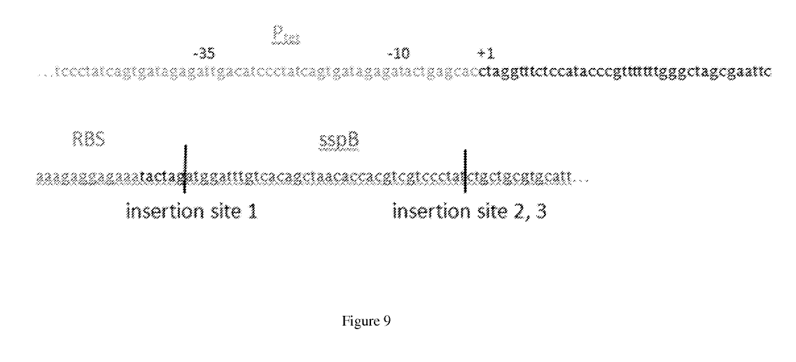

To screen IB1486-GA under batch conditions, working cultures comprising T12 medium with 15 g/L glucose were spiked with 100 μM IPTG during inoculation to induce the glucaric acid pathway enzymes. Additions of aTc were made at times varying from 0-30 hours after inoculation. FIG. 7 illustrates the yields and titers of glucaric acid observed after 48 hours. Glucose was fully consumed in all cultures at 48 hours, except for the culture with aTc addition at 12 hours, which still contained 6.6±0.1 g/L glucose after 48 hours. The highest titers of glucaric acid, 1.35 g/L (9.7% yield from glucose), were achieved with aTc addition at 24 hours, representing an 18% improvement in both yield and titer over the cultures without Pfk switching. As expected, switching after 24 hours resulted in lower titers, as more glucose had already been consumed for biomass and could not be redirected into glucaric acid production. Switching before 24 hours resulted in lower titers either due to incomplete consumption of glucose, as in the case of 12 hour aTc addition, or due to an “escape” phenotype. The escape phenotype correlated with rapid growth to higher cell densities (FIG. 8A) and an increase in Pfk activity (FIG. 8B). Studies of IB1863 have indicated that the increase in Pfk activity is likely linked to disruption of SspB expression, which is required for rapid Pfk-I degradation, through mutation or mobile element insertion (FIG. 9). Early addition of aTc results in high stress on the cell from a combination of limited glucose uptake and high protein expression for the glucaric acid pathway enzymes, which could result in more rapid selection for the escape phenotype.

Screening fed-batch conditions for timing of Pfk knockdown

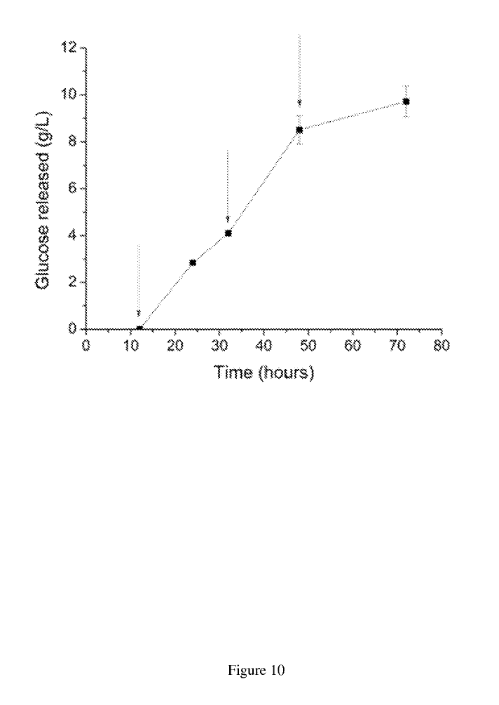

Fed-batch conditions were initially screened in T12 medium with 3 g/L free glucose and 12 g/L starch, with 100 μM IPTG added at inoculation to induce the glucaric acid pathway enzymes. Feeding commenced at 12 hours by the addition of 0.006 U/ml amyloglucosidase. As the starch hydrolysis rate declined with time, secondary additions of 0.006 U/ml and 0.012 U/ml amyloglucosidase were carried out at 36 and 48 hours, respectively, yielding the glucose release profile shown in FIG. 10. At the conclusion of the experiment, in addition to the initial 3 g/L glucose, an additional 9.7±0.7 g/L free glucose had been released in the cultures on average. To calculate the yield, unhydrolyzed starch was measured in the individual wells via the method outlined in Materials and Methods below.

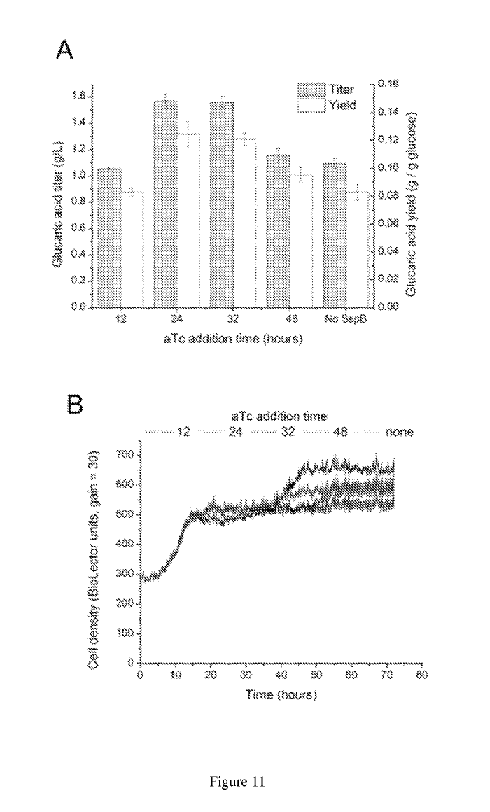

In this system, maximum titers of 1.56 g/L could be achieved with aTc addition at either 24 or 32 hours (FIG. 11A), a 42% improvement over cases withoutTc additions. The yields of glucaric acid were also improved by up to 50% with aTc addition at 24 hours, with a maximum yield of 12.4% on glucose (based on the available glucose added and hydrolyzed from starch). This improvement was larger than the batch condition, which may have been due to differences in the amount of glucose still available for consumption after the addition of aTc. Early aTc addition at 12 hours did not result in improved titers, and the shape of the growth curve was indicative of escape (FIG. 11B). The measurement of Pfk activity also showed activity recovery to levels at or above those where no aTc was added for this condition.

Validation of fed-batch results under altered feeding conditions

Consistent Improvements in Glucaric Acid Titers were Observed by Timed knockdown of Pfk activity under different conditions. To determine whether these improvements would be robust to moderate changes in culture conditions, a set of experiments was carried out under altered feeding conditions in the BioLector and also in shake flasks.

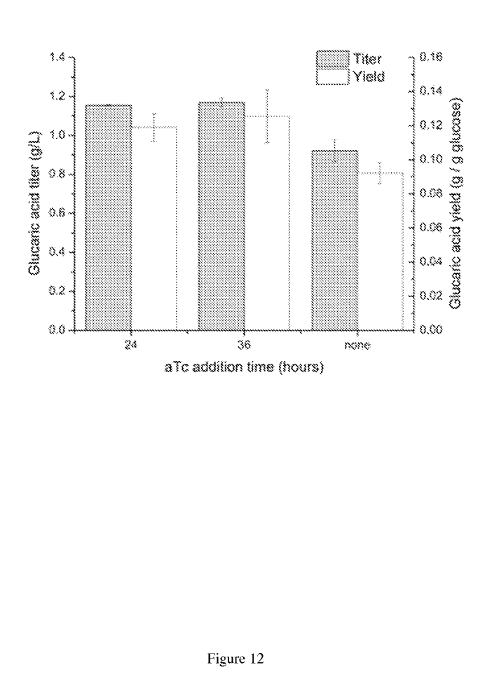

Previously, as the highest titers were achieved with Pfk knockdown at 24-32 hours, it was expected that growth up to that point could be carried out under either batch or fed-batch conditions without significant changes to the outcomes. An alternative feeding strategy was tested by increasing the initial free glucose to 5 g/L and commencing feeding at 24 hours from a reservoir of 10 g/L starch. This was initially tested in 48 well plates in the BioLector microbioreactor, with amyloglucosidase additions of 0.006 U/ml at 24 hours and 48 hours. The highest titers, 1.17 g/L, were achieved with aTc addition at 36 hours, a 27% improvement over those without aTc addition (FIG. 12). A maximum glucaric acid yield of 12.5% from glucose was also achieved with aTc addition at 36 hours (36% improvement over those without aTc addition). Although the titers were lower than in the original fed-batch screening, maximum yields were similar, as unhydrolyzed starch contents were higher under this feeding strategy. Ultimately, 9.7 g/L free glucose (initial dose and feeding) was available for conversion to glucaric acid, as compared to 12.7 g/L in the previous test.

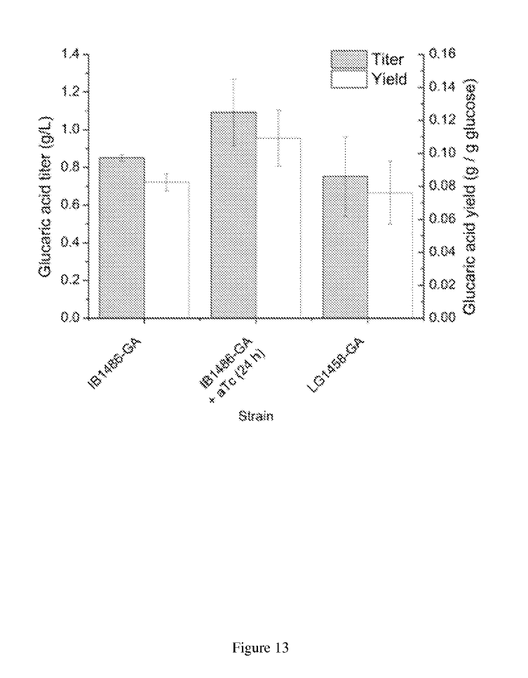

The starch fed-batch strategy was also tested in shake flasks, both for IB1486-GA and for LG1458-GA, a wild-type MG1655 background with only gudD and uxaC knockouts. The cultures contained 5 g/L free glucose and 10 g/L starch. Free glucose was measured at 18 hours, and since glucose was exhausted at that point, the amyloglucosidase addition was started at that time. Secondary additions were carried out at 40 and 48 hours. Despite the extra amyloglucosidase addition, starch hydrolysis was again poorer in this condition, and resulted in 10.1±0.5 g/L total free glucose available in the cultures on average. Baseline yields for IB1486-GA were still comparable with the previous tests and were similar to LG1458-GA. The addition of aTc for Pfk knockdown at 24 hours resulted in a 28% improvement in titers and a 32% improvement in yield over those without aTc additions, although variability was higher in the shake flasks than in previous testing in the BioLector (FIG. 13).

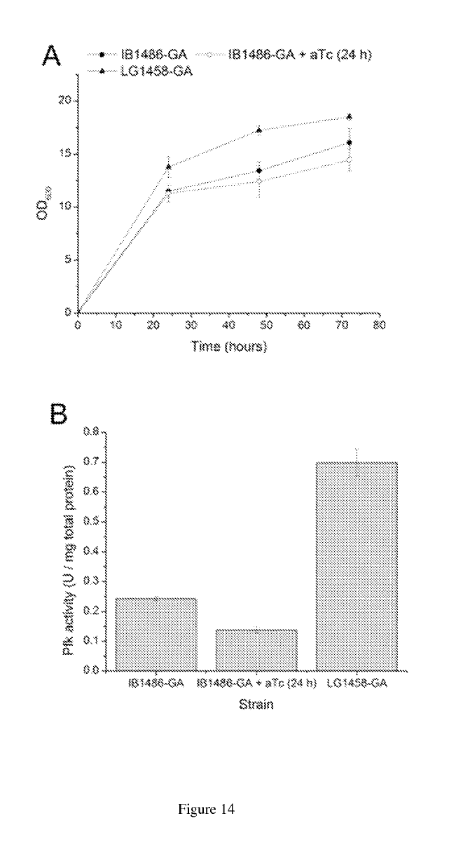

Growth (FIG. 14A) and Pfk activity measurements (FIG. 14B) showed trends similar to previous tests. However, a comparison of Pfk activity and the wild type LG1458-GA at 48 hours showed that IB1486-GA has significantly lower baseline activity than LG1458-GA before aTc addition. The aTc addition resulted in a 50% additional decrease in activity. The low baseline activity of IB1486-GA in T12 medium was unexpected, given that the parent strain, IB1863, always showed Pfk activity higher than that of the wild type in MOPS minimal medium with glucose (Brockman and Prather, 2015). While the mismatch in baseline activity did not cause a significant difference in titers between IB1486-GA and LG1458-GA under fed-batch conditions, this did affect batch performance strongly, as discussed below.

### Shake Flask Studies Under Batch Conditions

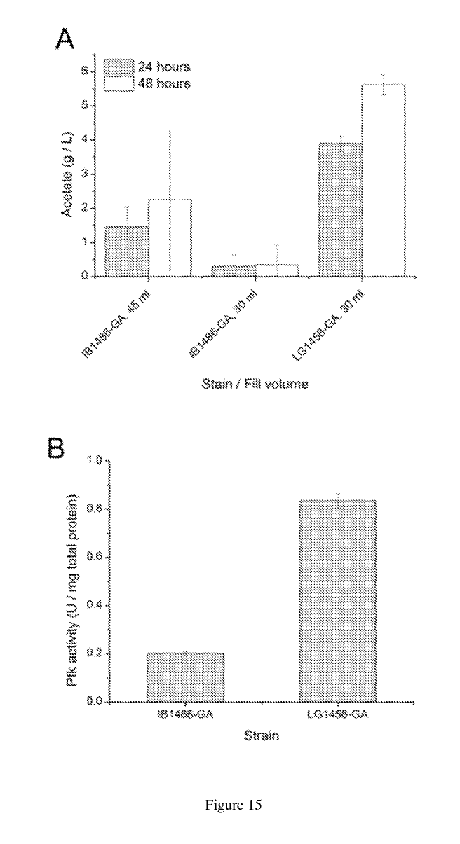

Glucaric acid production was also tested in shake flasks under batch conditions. In shake flasks, batch testing resulted in high baseline variability in titers; consequently, validation of improvements in the 20-40% range was difficult. However, the shake flask testing did provide some insight into the metabolism of IB1486-GA versus the wild-type strain LG1458-GA in the absence of any aTc addition. Significantly, under batch production conditions, acetate production varied greatly between the two strains; LG1458-GA produced much higher levels of acetate. The fill volume of flasks in batch testing appeared to have an effect on acetate production in IB1486-GA, potentially due to changes in aeration. In IB1486-GA, several cultures showed significant residual acetate at 48 hours in shake flasks with a 45 ml fill volume, while in previous testing with the BioLector, no acetate was observed at 48 hours. Testing at a lower fill volume (30 ml) resulted in reduced acetate accumulation at both 24 and 48 hours. A summary of the observed acetate production in IB1486-GA and LG1458-GA in shake flasks is presented in FIG. 15A. Pfk activity measurements at 24 hours in IB1486-GA and IB1458-GA (FIG. 15B) showed that, as in the fed-batch case, Pfk activity in LG1458-GA was significantly higher, likely leading to metabolic overflow and acetate production.

Glucaric acid production was also very poor for LG1458-GA under batch conditions, with titers below the limit of detection in T12+15 g/L glucose (30 ml fill volume). LG1458-GA showed incomplete glucose consumption as well, with 3.0±0.1 g/L remaining at 48 hours. Although titers in IB1486-GA showed high variability, glucaric acid production was clear in all samples with T12+15 g/L glucose, with measured titers of 0.9±0.3 g/L glucaric acid in shake flasks with 30 ml fill volume. Glucose was also completely consumed in the cultures.

In contrast to the batch condition, under the starch hydrolysis (glucose feeding) condition shown in FIG. 13, glucaric acid titers of 0.75±0.2 g/L were achieved in LG1458-GA, more comparable to the 0.85±0.02 g/L produced in IB1468-GA without aTc addition, and any acetate produced was fully consumed by the conclusion of the experiment for both strains. Yields were also more comparable between the two strains under glucose feeding; 7.6% for LG1458-GA and 8.3% for IB1458-GA. An advantages of the fed-batch condition is the elimination of acetate buildup and carbon loss to acetate formation. Additional changes in metabolism, such as upregulation of genes associated with sugar transport (Franchini and Egli, 2006; Raman et al., 2005), could also be favorably changing relative metabolite pools. While a fed-batch production strategy provided a significant benefit for LG1458-GA, IB1468-GA does not benefit as strongly from slower glucose feeding, likely due to the fact that acetate production is already much lower in this strain. Pfk activity may also be low enough in this strain that other changes in metabolism related to glucose limitation are not significant.

Although a static condition of low Pfk activity can clearly be tolerated in T12 medium and can provide a benefit in glucaric acid production under batch conditions, the previous screening work above indicated that there is a limit to the possible gain in this manner, as further knockdown of Pfk activity by aTc addition at inoculation resulted in poor growth and eventual “escape” of the culture. A ApfkA ApfkB double knockout strain, IB2255, was tested to assess the productivity that could be expected in T12 medium under batch conditions with no Pfk activity. After 48 hours in T12 with 15 g/L glucose, 0.18±0.01 g/L glucaric acid was produced, significantly lower than the amounts produced in IB1486-GA in shake flasks in batch conditions (0.9±0.3 g/L). To maximize productivity in a 48 hour batch, the culture must be provided with higher Pfk activity for a period of time.

### Discussion

Results with IB1486-GA indicate that the dynamic control of Pfk activity can be utilized to improve titers of glucaric acid. The system discussed above was applicable for use with a semi-defined medium under both batch and simulated fed-batch conditions. While Gains in Titers were Consistent Across Multiple Conditions, the Maximum Gains were smaller than those observed previously for myo-inositol production in glucose minimal medium (Brockman and Prather, 2015). Previous work with myo-inositol production in glucose minimal medium showed that switching at low cell density was optimal for the largest gains in titer. In T12 medium, the earlier switching times resulted in more rapid escape and little time for the conversion of glucose to glucaric acid, perhaps due to the greater expression burden of the complete glucaric acid pathway and the higher availability of nutrients in T12 that “escapers” could use to rapidly grow and overtake the population. The later switching times resulted in a higher usage of glucose for biomass formation; therefore, the amount of glucose processed after switching to production mode was relatively low.

Activity of the downstream enzymes in the glucaric acid pathway is a potential limitation, but it does not appear that the activity of MIOX, a bottleneck under some other conditions, was limited the overall pathway yield in the tested medium, as a minimal buildup of myo-inositol was observed in the cultures. However, forming a balance of expression between the three pathway enzymes could be an issue, since high levels of INO1 expression are required for any myo-inositol to be produced for further conversion (Moon et al., 2009). Reductions in INO1 expression upon the expression of other enzymes in the glucaric acid pathway would be expected to limit maximum fluxes into the pathway, also limiting the glucose that could be effectively redirected in a given time period.

IB1486-GA showed titers that were comparable with a wild-type control strain under fed-batch conditions and superior under batch conditions, indicating the genetic modifications required for control of Pfk activity were not detrimental to baseline glucaric acid production and could potentially be transferred into high-performing strains as well. Although the baseline Pfk activity was low in T12 medium, it was still sufficient for rapid growth without excessive overflow metabolism. The K strains showed more consistent success, which led to the initial construction of the Pfk-I control system in that background, but additional improvements in glucaric acid titer, both with and without Pfk knockdown, can likely be achieved by transferring the genetic modifications of IB1486 to an E. coli B strain. In previous work, wild-type BL21 outperformed MG1655 containing the same pathway genes (Moon et al., 2009; Raman et al., 2014; Shiue et al., 2015).

### Conclusions

Glucaric acid titers and yields could be improved under multiple culture conditions through the timed knockdown of Pfk activity, with maximum improvements of up to 42% observed. In the absence of aTc, the switchable strain IB1486 shows titers comparable to or above those observed with wild-type MG1655, indicating the genetic modifications in IB1486 do not result in the degradation of baseline performance and could potentially be applied to high-performing strains for increases in titer. Optimization of strain background and pathway enzyme expression levels may lead to both higher baseline titers and to greater gains from dynamic control of Pfk activity.

### Materials and Methods for Example 2

### Strains and Plasmids

E. coli strains and plasmids used in this study are listed in Table 2. To eliminate catabolism of glucaric acid and the pathway intermediate glucuronic acid in strain IB1863, knockouts of gudD and uxaC were carried out via sequential P1 transduction from Keio collection donor strains (Baba et al., 2006). The kanamycin resistance cassette was removed after each transduction via expression of FLP recombinase from pCP20 (Datsenko and Wanner, 2000). The λDE3 lysogen was integrated into this strain using a λDE3 Lysogenization Kit (Novagen, Darmstadt, Germany), generating strain IB1486. To generate the ApfkA control strain IB2255, serial P1 transductions were also carried out in IB1379 to knock out pfkA, gudD, and uxaC, and the λDE3 lysogen was integrated as described above. Construction of plasmids for production of glucaric acid, pRSFD-IN-MI and pTrc-udh, was described previously (Moon et al., 2009; Yoon et al., 2009).

### Culture Medium and Conditions

For plasmid preparation and genetic manipulations, strains were cultured in Luria-Bertani (LB) medium at either 30° or 37° C. Temperature sensitive plasmids were cured at 42° C. Glucaric acid production experiments were carried out in T12 medium containing 7.5 g/L yeast extract, 7.5 g/L soytone, 7 g/L Na₂HPO₄, 3 g/L KH₂PO₄, 0.5 g/L NaCl, 3 g/L (NH₄)₂50₄, 4 mM MgSO₄, 100 μg/ml carbenicillin, 50 μg/ml kanamycin, and the indicated amount of glucose and/or soluble starch (Sigma-Aldrich S9765) plus amyloglucosidase (Sigma-Aldrich A7095). For experiments in the BioLector (m2p-labs, Baesweiler, Germany), starter cultures were incubated in culture tubes at 30° C. and 250 rpm overnight in T12 supplemented with 10 g/L glucose and diluted 1:100 into working cultures. Working cultures were incubated at 30° C., 1200 rpm (3 mm orbit), and 80% relative humidity in the BioLector. A working volume of 1 ml was used in the BioLector 48-well flower plate, sealed with gas-permeable sealing foil with evaporation reduction (m2p-labs). To induce expression of enzymes required for glucaric acid production, 100 μM β-D-1-thiogalactopyranoside (IPTG) was added to working cultures at inoculation. For induction of SspB in strain IB1486 to knock down Pfk activity, 100 ng/ml anhydrotetracycline (aTc) was added at the times indicated in the Results section. At the indicated time points, the contents of the sample well were removed for measurement of glucaric acid production and residual glucose levels.

For experiments in shake flasks, starter cultures were grown to mid-exponential phase (OD₆₀₀ ˜5) in 250 ml baffled flasks containing 30 ml T12+10 g/L glucose and used to inoculate 30 ml working cultures to a starting OD₆₀₀=0.05. Working cultures were incubated in 250 ml baffled shake flasks at 30° C., 80% humidity, and 250 rpm. IPTG (100 μM) was added at inoculation and aTc (100 ng/ml) was added at the indicated time points. Flasks were sampled periodically for measurement of optical density, as well as for HPLC and biomass samples. Samples from all experiments were stored at −20° C. until analysis.

### Measurement of Extracellular Metabolites and Starch

Glucose, glucaric acid, acetate, and myo-inositol levels were quantified by high performance liquid chromatography (HPLC) on an Agilent 1100 or 1200 series instrument (Santa Clara, Calif.) with an Aminex HPX-87H anion exchange column (300 mm by 7.8 mm; Bio-Rad Laboratories, Hercules, Calif.). Sulfuric acid (5 mM) at a flow rate of 0.6 mL/min was used as the mobile phase. Compounds were quantified from 10 μL sample injections using refractive index (glucose, myo-inositol, glucaric acid, acetate) and diode array detectors (glucaric acid, 210 nm). Column and refractive index detector temperatures were held at 65° C. and 35° C., respectively.

To quantify the amount of starch hydrolysis in fed-batch samples, samples were split at collection. Half of the sample was centrifuged at 15000×g for 15 minutes and used for HPLC analysis as described above. The remaining portion of the sample was treated with 15 U/ml amyloglucosidase for 15 minutes at room temperature for full hydrolysis of remaining starch. After treatment, the sample was centrifuged for 5 minutes at 15000×g and the glucose concentration in the supernatant was measured using a YSI 2900 Biochemistry Analyzer (YSI Life Sciences, Yellow Springs, Ohio). The difference between the glucose content measured in the fully hydrolyzed sample and the glucose content measured via HPLC in the sample without an additional hydrolysis step was used to calculate the content of unhydrolyzed starch.

The maximum amount of glucose that could be liberated from starch in the medium was determined by full hydrolysis of the starting medium with amyloglucosidase. To calculate glucose utilized by the cell, the amount of free glucose and the amount glucose generated from full hydrolysis of residual starch in a sample were subtracted from the maximum amount available in the medium. This value for consumed glucose was then used in the calculation of glucaric acid yield from glucose.

### Phosphofructokinase Activity Measurements

All enzymatic activity assays were carried out on crude lysates. For preparation of lysates, samples of 5-10 ml of cell culture were collected, frozen at −80° C., and then resuspended in 50 mM Tris-HCl, pH 7.4 (0.25-1 ml, depending on cell density). Cells were lysed via bead beating for 5 minutes and lysates clarified by centrifugation at 15,000×g for 15 minutes. Phosphofructokinase activity was assayed using a protocol adapted from Kotlarz and Buc (Kotlarz and Buc, 1982; Kotlarz et al., 1975). The assay mixture consisted of 0.1 M Tris-HCl (pH 8.2), 10 mM MgCl2, 1 mM ATP, 0.2 mM β-NADH, 1 mM fructose-6-phosphate (F6P), 1 mM NH4Cl, 0.01% Triton X-100, 0.83 U aldolase, 0.42 U triosephosphate isomerase, and 0.42 U glycerophosphate dehydrogenase. Reaction progress was followed by measurement of absorbance at 340 nm. One unit of Pfk activity was defined as the amount required to convert 1.0 μmole of ATP and D-fructose 6-phosphate to ADP and fructose 1,6-bisphosphate per minute at pH 8.2 and room temperature.

### Supplementary Methods

Cultures for analysis of escape were carried out at 30° C. in a modified MOPS medium containing 10 g/L D-glucose, 3 g/L NH₄Cl, 1 g/L K₂HPO₄, 2 mM MgSO₄, 0.1 mM CaCl₂, 40 mM MOPS, 4 mM tricine, 50 mM NaCl, 100 mM Bis-Tris, 134 μM EDTA, 31 μM FeCl₃, 6.2 μM ZnCl₃, 0.76 μM CuCl₂, 0.42 μM CoCl₂, 1.62 μM H₃BO₃, and 0.081 μM MnCl₂. Seed cultures were initiated using a 1:100-1:500 dilution from LB cultures and were grown at 30° C. for 18-24 hours in modified MOPS, until mid-exponential phase was reached. Working cultures of 50 ml in 250 ml baffled shake flasks were inoculated to OD₆₀₀=0.05 from seed cultures.

Any system relying on arrest of cellular growth is naturally applying a strong selection for compensatory mutations. In this particular system, any mutation that prevents the degradation of Pfk-I after aTc additional will provide a relative fitness advantage, and those cells will eventually become the predominant population. Possible points for mutation include the degradation tag on pfkA, the coding sequence or promoter for sspB, and the coding sequence or promoter for clpX and clpP. To determine if these mutations did occur in escaped cells, strain IB1863 was grown in triplicate in modified MOPS minimal medium with glucose and 100 ng/ml aTc was added at OD 0.25. The cells showed very slow growth for 48 hours, but between 48 and 72 hours, grew rapidly and consumed all available glucose. At that time, samples from all three flasks were streaked onto LB agar. New cultures were also started from these flasks, and they no longer showed growth arrest in response to aTc addition, indicating the observed growth was not due to breakdown of inducer.

Colony PCR was then carried out on two colonies isolated from each flask using primers that amplified the regions containing pfkA(DAS+4), clpX, and the sspB expression cassette. In these 6 colonies, clpX and pkfA(DAS+4) amplification resulted in the expected band sizes and sequencing of the PCR products also returned the correct sequences. Amplification of the sspB cassette resulted in 4 cases of PCR products with a larger than expected band size and 2 cases of no amplification. Three of the recovered PCR products were sent for sequencing and revealed an insertion that disrupted up to 36 base pairs of sspB and separated it from the tet promoter (FIG. 9). A BLAST search revealed that the insertion sequence matched that found in the IS2 insertion element.

To improve the stability of such constructs, it could be beneficial to utilize a modified strain background with mobile genetic elements deleted. Fluctuation assays comparing one such reduced genome strain to MG1655 showed that 24% of loss of function mutations in MG1655 were caused by IS transpositions, and those could be eliminated in the IS-free strain MD41 (Pósfai et al., 2006). An alternative using the existing strain background could be to add an essential gene or toxin/antitoxin system to the sspB expression cassette to limit the possible locations where transposable elements could successfully be inserted without causing cell death.

### Tables of Example 2

TABLE 2
Strains and plasmids

| Strain/plasmid | Genotype | Reference/source |
|---|---|---|
| Strains | | |
| LG1458 | MG1655(DE3) ΔgudD ΔuxaC | Prather Lab |
| IB1863 | MG1655 ΔendA Δzwf ΔpfkB ΔsspB pfkA::114- | (Brockman and Prather, |
| | pfkA(DAS + 4) HK022::tetR-Ptet-sspB | 2015) |
| IB1379 | MG1655 ΔendA Δzwf ΔpfkB | (Brockman and Prather, |
| | | 2015) |
| IB1486 | MG1655(DE3) ΔendA Δzwf ΔpfkB ΔsspB pfkA::114- | This study |
| | pfkA(DAS + 4) HK022::tetR-Ptet-sspB ΔgudD ΔuxaC | |
| IB2255 | MG1655(DE3) ΔendA Δzwf ΔpfkB ΔpfkA ΔgudD | This study |
| | ΔuxaC | |
| JW2758-5 | F−, Δ(araD-araB)567, ΔlacZ4787(::rrnB-3), λ⁻, | (Baba et al., 2006) |
| | ΔgudD785::kan, rph-1, Δ(rhaD-rhaB)568, hsdR514 | |
| JW3887-1 | F−, Δ(araD-araB)567, ΔlacZ4787(::rrnB-3), λ⁻, | (Baba et al., 2006) |
| | ΔpfkA775::kan, rph-1, Δ(rhaD-rhaB)568, hsdR514 | |
| JW3603-2 | F−, Δ(araD-araB)567, ΔlacZ4787(::rrnB-3), λ⁻, | (Baba et al., 2006) |
| | ΔuxaC782::kan, rph-1, Δ(rhaD-rhaB)568, hsdR514 | |
| IB1486-GA | IB1486/pRSFD-IN-MI/pTrc-udh | This study |
| LG1458-GA | LG1458/pRSFD-IN-MI/pTrc-udh | This study |
| IB2255-GA | IB2255/pRSFD-IN-MI/pTrc-udh | This study |
| Plasmids | | |
| pCP20 | Rep^{a}, Amp^{R}, Cm^{R}, FLP recombinase expressed by λ pᵣ | CGSC #7629 |
| | under control of λ cI857 | |
| pRSFD-IN-MI | pRSR1030 ori, lacI, Kan^{R}, INO1 (S. cerevisiae) and | (Moon et al., 2009) |
| | MIOX (M. musculus) expressed under control of T7 | |
| | promoter | |
| pTrc-udh | pBR322 ori, lacI, Amp^{R}, Udh (P. syringae) expressed | (Yoon et al., 2009) |
| | under control of Trc promoter | |

### REFERENCES FOR EXAMPLE 2

Anesiadis, N., Cluett, W. R., Mahadevan, R., 2008. Dynamic metabolic engineering for increasing bioprocess productivity. Metab Eng. 10, 255-66.
Baba, T., Ara, T., Hasegawa, M., Takai, Y., Okumura, Y., Baba, M., Datsenko, K. A., Tomita, M., Wanner, B. L., Mori, H., 2006. Construction of Escherichia coli K-12 in-frame, single-gene knockout mutants: the Keio collection. Mol Syst Biol. 2, 2006 0008.
Brockman, I. M., Prather, K. L., 2015. Dynamic knockdown of E. coli central metabolism for redirecting fluxes of primary metabolites. Metab Eng. 28, 104-13.
Datsenko, K. A., Wanner, B. L., 2000. One-step inactivation of chromosomal genes in Escherichia coli K-12 using PCR products. Proceedings of the National Academy of Sciences of the United States of America. 97, 6640-5.
Franchini, A. G., Egli, T., 2006. Global gene expression in Escherichia coli K-12 during short-term and long-term adaptation to glucose-limited continuous culture conditions. Microbiology. 152, 2111-2127.
Gadkar, K. G., Doyle III, F. J., Edwards, J. S., Mahadevan, R., 2005. Estimating optimal profiles of genetic alterations using constraint-based models. Biotechnol Bioeng. 89, 243-51.
Kiely, D. E., Chen, L., Glucaric acid monoamides and their use to prepare poly(glucaramides). Google Patents, 1994.
Kogure, T., Wakisaka, N., Takaku, H., Takagi, M., 2007. Efficient production of 2-deoxy-scyllo-inosose from d-glucose by metabolically engineered recombinant Escherichia coli. Journal of Biotechnology. 129, 502-509.
McGinness, K. E., Baker, T. A., Sauer, R. T., 2006. Engineering controllable protein degradation. Mol Cell. 22, 701-7.
Mehltretter, C. L., Rist, C. E., 1953. Sugar Oxidation, Saccharic and Oxalic Acids by the Nitric Acid Oxidation of Dextrose. Journal of Agricultural and Food Chemistry. 1, 779-783.
Moon, T. S., Dueber, J. E., Shiue, E., Prather, K. L. J., 2010. Use of modular, synthetic scaffolds for improved production of glucaric acid in engineered E. coli. Metabolic Engineering. 12, 298-305.
Moon, T. S., Yoon, S. H., Lanza, A. M., Roy-Mayhew, J. D., Prather, K. L., 2009. Production of glucaric acid from a synthetic pathway in recombinant Escherichia coli. Appl Environ Microbiol. 75, 589-95.
Pósfai, G., Plunkett, G., Fehér, T., Frisch, D., Keil, G. M., Umenhoffer, K., Kolisnychenko, V., Stahl, B., Sharma, S. S., de Arruda, M., Burland, V., Harcum, S. W., Blattner, F. R., 2006. Emergent Properties of Reduced-Genome Escherichia coli. Science. 312, 1044-1046.
Raman, B., Nandakumar, M. P., Muthuvijayan, V., Marten, M. R., 2005. Proteome analysis to assess physiological changes in Escherichia coli grown under glucose-limited fed-batch conditions. Biotechnology and Bioengineering. 92, 384-392.
Raman, S., Rogers, J. K., Taylor, N. D., Church, G. M., 2014. Evolution-guided optimization of biosynthetic pathways. Proceedings of the National Academy of Sciences. 111, 17803-17808.
Scalcinati, G., Knuf, C., Partow, S., Chen, Y., Maury, J., Schalk, M., Daviet, L., Nielsen, J., Siewers, V., 2012. Dynamic control of gene expression in Saccharomyces cerevisiae engineered for the production of plant sesquitepene alpha-santalene in a fed-batch mode. Metab Eng. 14, 91-103.
Shiue, E., Brockman, I. M., Prather, K. L., 2015. Improving product yields on D-glucose in Escherichia coli via knockout of pgi and zwf and feeding of supplemental carbon sources. Biotechnol Bioeng. 112, 579-87.
Shiue, E., Prather, K. L., 2014. Improving D-glucaric acid production from myo-inositol in E. coli by increasing MIOX stability and myo-inositol transport. Metab Eng. 22, 22-31.
Smith, T. N., Kiely, D. E., Kramer-Presta, K., Corrosion inhibiting composition. Google Patents, 2012.
Solomon, K. V., Sanders, T. M., Prather, K. L., 2012. A dynamic metabolite valve for the control of central carbon metabolism. Metab Eng. 14, 661-71.
Soma, Y., Tsuruno, K., Wada, M., Yokota, A., Hanai, T., 2014. Metabolic flux redirection from a central metabolic pathway toward a synthetic pathway using a metabolic toggle switch. Metab Eng. 23, 175-84.
Torella, J. P., Ford, T. J., Kim, S. N., Chen, A. M., Way, J. C., Silver, P. A., 2013. Tailored fatty acid synthesis via dynamic control of fatty acid elongation. Proceedings of the National Academy of Sciences. 110, 11290-11295.
Werpy, T., Petersen, G., Top value added chemical from biomass, volume 1-results of screening for potential candidates from sugars and synthetic gas. U.S. Department of Energy, Washington, D.C., 2004.
Williams, T. C., Averesch, N. J. H., Winter, G., Plan, M. R., Vickers, C. E., Nielsen, L. K., Krömer, J. O., 2015. Quorum-sensing linked RNA interference for dynamic metabolic pathway control in Saccharomyces cerevisiae. Metabolic Engineering. 29, 124-134.
Yoon, S.-H., Moon, T. S., Iranpour, P., Lanza, A. M., Prather, K. J., 2009. Cloning and Characterization of Uronate Dehydrogenases from Two Pseudomonads and Agrobacterium tumefaciens Strain C58. Journal of Bacteriology. 191, 1565-1573.

### Example 3: Cell Density-Based Autonomous Control of Pfk-I Expression

### Abstract

Dynamic control of native enzymes in cellular metabolism offers opportunities to better balance growth and small-molecule production, as well as to avoid buildup of intermediates. Currently, most dynamic systems rely on addition of an inducer to the culture medium in order to trigger alterations in enzyme level. This work demonstrates that systems based on both nutrient starvation (phosphate starvation) and quorum sensing (esa system) can be used in E. coli to control expression levels of phosphofructokinase-I, a key enzyme in glycolysis. In these modified cells, Pfk activity drops and growth is autonomously arrested upon reaching a critical cell density, without addition of an exogenous inducer. It was further demonstrated that yields and titers of a heterologous product, myo-inositol, could be improved through use of this system, without requiring any outside intervention during the course of the fermentation.

### Introduction

Balancing native metabolism with newly introduced or overexpressed small-molecule production pathways is a significant challenge in the development of new bioprocesses. Improved steady-state flux distributions can be generated through combinatorial promoter replacement and gene knockouts (Santos and Stephanopoulos, 2008), but when the pathway of interest is in direct competition with growth, this may result in reductions in growth rate that ultimately limit productivity. In these cases, a dynamic strategy of switching between a growth mode with high biomass formation and a production mode with greater flux into product formation could maximize productivity in a fixed batch time (Anesiadis et al., 2008; Anesiadis et al., 2013; Gadkar et al., 2005).

Provided herein is a system for dynamic control of phosphofructokinase-I (Pfk-I) levels in the cell, which can be used to increase yields and titers of products derived from glucose-6-phosphate (G6P) (Brockman and Prather, 2015). Rather than manually monitoring biomass and adding inducer (e.g., to control Pfk-1 levels), autonomous triggers in the cell were developed, where Pfk-I levels are regulated after formation of a target amount of biomass.

A number of systems based on nutrient starvation promoters such as phoA and trp have been used for applications requiring cell density-dependent or delayed protein expression in E. coli (Laird et al., 2005; Swartz, 2001; Yoon et al., 1996). In Saccharomyces cerevisiae, the HXT1, HXT2, and GAL1/GAL10 promoters have been successfully used to link control of gene expression to glucose concentrations in the medium (Scalcinati et al., 2012; Xie et al., 2015). Limitation in a key nutrient can alter fluxes and inherently limit the amount of biomass that can be formed, and this strategy has been used for maximization of product titers in the absence of any additional starvation-based protein expression strategy (Johansson et al., 2005; Youngquist et al., 2013; Zhu et al., 2008). However, nutrient starvation also induces a wide variety of regulatory responses in the cell, some of which may be undesirable for product formation.

As an alternative, quorum sensing (QS) systems are a mechanism for carrying out cell-density dependent processes, and have been used synthetically for applications such as timed induction of recombinant proteins (Tsao et al., 2010), timed lysis (Saeidi et al., 2011), and balancing of different cell populations (Balagadde et al., 2008). The esa quorum sensing system from Pantoea stewartii could be used as a platform for fine-tuning cell-density dependent gene expression. Two promoters with opposite responses to binding by the quorum sensing transcription factor EsaR are known. EsaR binds DNA in the absence of its cognate autoinducer, 3-oxohexanoyl-homoserine-lactone (3OC6HSL) (Minogue et al., 2002). The P_{esaS} promoter is activated by EsaR binding (Schu et al., 2009), while the P_{esaR} promoter is repressed by EsaR binding (Minogue et al., 2002). Additionally, a number of variants of the EsaR transcription factor have been developed by directed evolution, which show varying levels of binding affinity to 3OC6HSL (Shong et al., 2013), providing a secondary method to tune timing of promoter derepression or deactiviation, beyond only control of 3OC6HSL synthase (EsaI) expression level.

Utilizing components of the esa quorum sensing system, a new autonomous system for control of Pfk-I levels was developed. Uniquely, this system requires no outside intervention, but rather relies on the cells themselves for synthesis of the autoinducer 3OC6HSL, with Pfk activity knockdown and growth arrest occurring when the threshold level of 3OC6HSL is reached. Provided herein are a series of host strains with varying levels of 3OC6HSL synthase expression, allowing a host strain to be selected with a desired time of Pfk-I switching. Utilizing this system for the previously tested application of myo-inositol production, an increase in titers of 30% over a host strain with a native pfkA promoter was achieved on glucose as a sole carbon source.

### Phosphate Starvation-Inducible Knockdown of Pfk Activity

The use of phosphate starvation to drive SspB expression, and a corresponding reduction in Pfk activity, is a unique strategy. Phosphate is an essential nutrient and phosphate starvation strategies have already been demonstrated to improve yields for a number of products, such as shikimic acid and fatty acids (Johansson et al., 2005; Youngquist et al., 2013). Additionally, phosphate feeding may achieve cycling in the Pfk activity level, unlike in the case of inducer addition, where once added, the inducer cannot be easily removed to stop SspB expression and allow the recovery of Pfk activity. Thus, phosphate starvation provides an autonomous, reversible method for controlling the shift between growth (high Pfk activity) and production (low Pfk activity) modes. The phoA promoter has previously been shown in the literature to exhibit strong induction upon phosphate starvation (Shin and Seo, 1990) and was thus initially selected for evaluation.

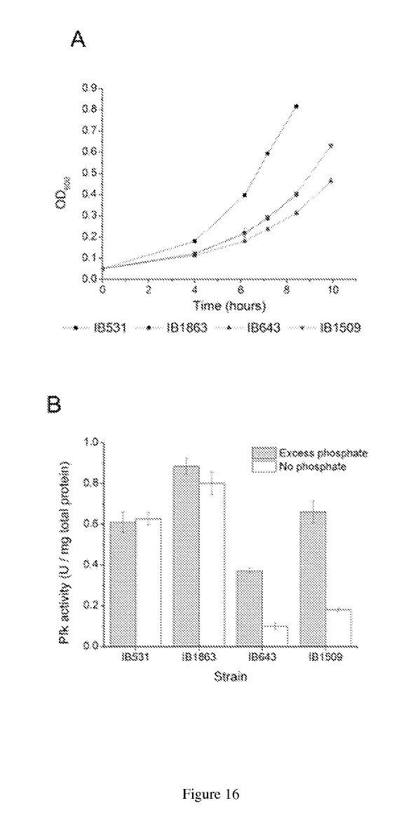

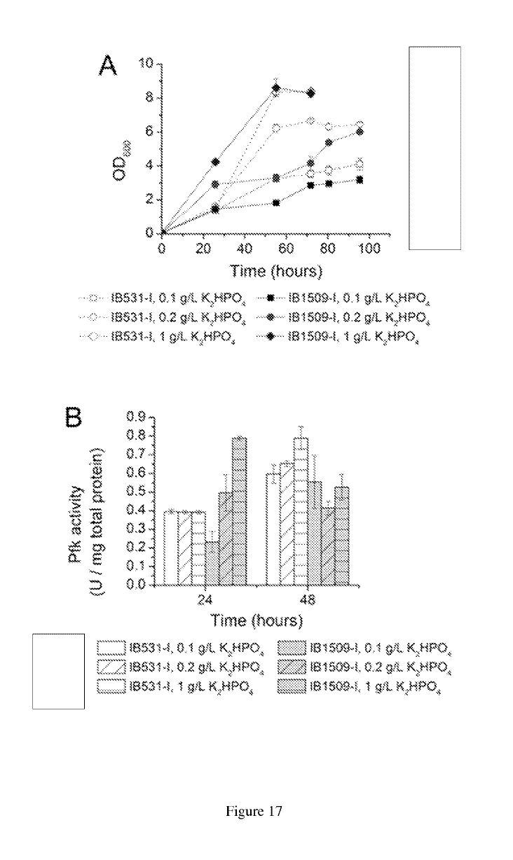

To verify that the phoA promoter amplified from the E. coli genome would exhibit the desired induction behavior in media of interest, the promoter construct was used to drive GFP expression from a plasmid, and strong induction was seen in glucose minimal medium under phosphate starvation conditions (data not shown). Strains IB643 and IB1509 were then constructed to test the potential applicability of this strategy for SspB expression. Each strain has a genomically-integrated cassette for the expression of sspB driven by the phoA promoter. The strain backgrounds are identical except for the constitutive promoter driving Pfk expression. Two promoter variants were tested (apFAB 114 and apFAB104). It was shown that apFAB104 resulted in higher expression of Pfk than apFAB114 above, and it may balance Pfk expression when expressing SspB from promoters which were leakier than the tet promoter. The growth of IB643 and IB1509 was measured in modified MOPS minimal medium with 10 g/L glucose and excess phosphate. The growth rate of IB1509 was similar to IB1863, where SspB expression was driven by the tet promoter, while IB643 was slightly slower (FIG. 16A). To test the effect of phosphate starvation on Pfk activity, cultures were spun down and transferred from the excess phosphate condition to phosphate-free modified MOPS minimal medium with 10 g/L glucose. Pfk activity was measured after one hour in phosphate starvation. IB1509 and IB643 showed a strong decline in Pfk activity in response to phosphate starvation, while the wild-type and Pₜₑᵣ-sspB controls did not (FIG. 16B). IB1509 showed baseline activity and growth closest to the IB531 control and was selected for further testing.

To test whether the decline in Pfk activity upon phosphate starvation could be used to improve yields of MI, strain IB1509 was transformed with pTrc-INO1. MI production in this strain was compared to MI production in the wild-type strain, IB5314, in modified MOPS minimal medium supplemented with 10 g/L glucose and 0.1, 0.2, or 1 g/L K₂HPO₄. At 0.1 or 0.2 g/L K₂HPO₄, the available phosphate in the medium was exhausted before the glucose was fully consumed, limiting the formation of biomass. The excess glucose cannot be used to form biomass, but it is consumed and converted into other products as long as the cells remain viable under the nutrient-limited condition.

The growth profiles for IB5314 and IB15094 (FIG. 17A) show that biomass formation is limited in both strains at 0.1 g/L and 0.2 g/L K₂HPO₄. The growth profile for IB1509-I shows an earlier plateau in biomass formation under phosphate starvation conditions, and then the slow formation of biomass up to wild-type levels. This corresponds with the expected behavior for the strain: as phosphate levels fall below the threshold of phoA induction, SspB is expressed and Pfk activity declines, limiting glycolytic flux and the ability to form biomass. Activity measurements in samples taken from the cultures showed that Pfk activity in the cultures with 0.1 g/L K₂HPO₄ had already fallen below the wild-type levels by 24 hours (FIG. 17B). At 48 hours, some recovery of Pfk activity was observed in the cultures with the lowest phosphate level; however, all IB1509-I cultures still showed activity below that of the wild-type.

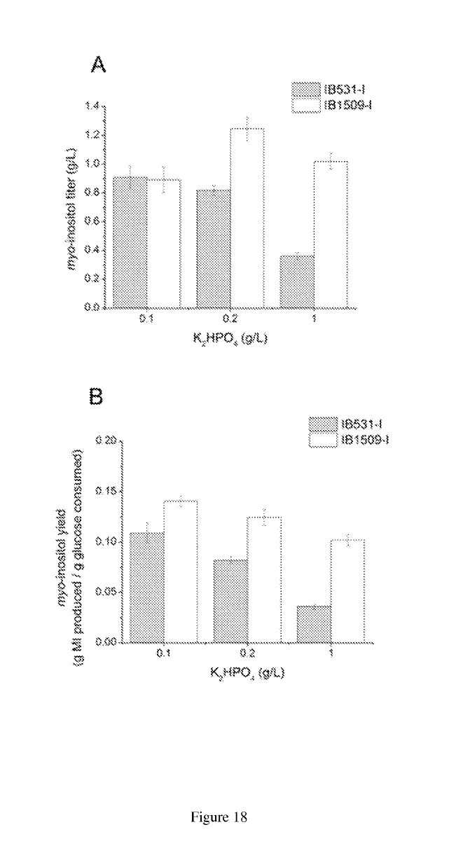

FIG. 18A shows the titers, and FIG. 18B, the yields, of MI observed in IB1509-I and IB531-I at the conclusion of the experiment (96 hours). The titers of MI were improved by phosphate starvation in both strains, not just in IB1509-I. This may be due to the natural limitation of glycolytic flux during phosphate starvation or the upregulation of endogenous phosphatases that dephosphorylate myo-inositol-1-phosphate to MI more rapidly, helping to pull flux through INO1. IB1509-I did show improved yields and titers over IB531-I; however, this was true under conditions of excess phosphate as well as phosphate starvation.

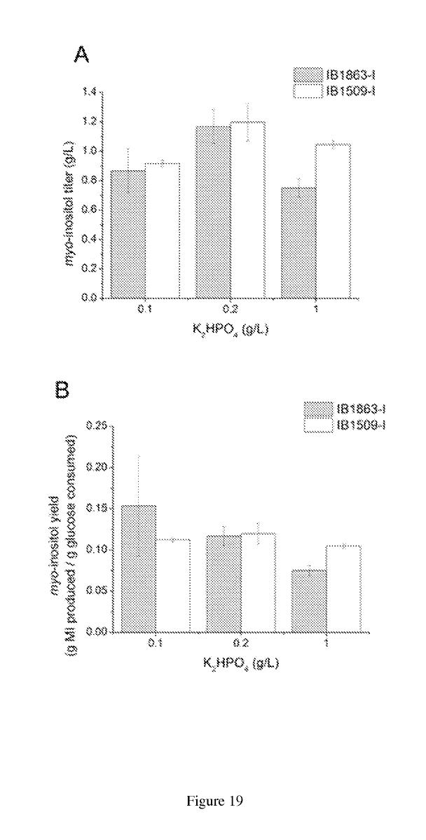

Realizing that the improvement in titers could be driven, at least partially, by the deletion of zwf in IB1509-I, which is not present in IB531-I, MI production in IB1509-I was also compared to that in IB1863-I under phosphate starvation. IB1863-I has the same genetic modifications as IB1509-I, except that expression of SspB is driven by a tet promoter rather than the phoA promoter. Titers (FIG. 19A) and yields (FIG. 19B) were very similar between the two strains across all conditions, indicating the effect of Pfk knockdown at phosphate starvation on the MI titer was weak compared to the broader effect of phosphate starvation alone. This may be due to the fact that flux into glycolysis is already relatively low under phosphate starvation conditions.

While phosphate starvation-based SspB induction did not show a significant impact on MI production in a batch system, it could have an effect in fed-batch applications. The use of the PphoA-based system creates an inherent lag in the recovery of Pfk activity because when phosphate is again added to the system to shut off SspB expression, the SspB protein already present in the cell must be diluted before Pfk degradation ceases. Thus, while wild-type cells can rapidly return to full metabolism upon phosphate addition after starvation, IB1509 has a slower transition.

### Arabinose-Inducible Knockdown of Pfk Activity

As an alternative variant of the system, arabinose-inducible expression of SspB was explored to process sugar mixtures derived from cellulosic biomass. In the sugar mixtures, glucose will be present, along with pentoses such as arabinose and xylose. Ideally, catabolite repression from glucose would prevent the induction of SspB until that sugar has been fully consumed. Glucose should be rapidly consumed rapidly for biomass formation, and upon glucose exhaustion, SspB should be induced and then the Pfk knockdown redirects the carbon flux from xylose and arabinose consumption into glucaric acid production.

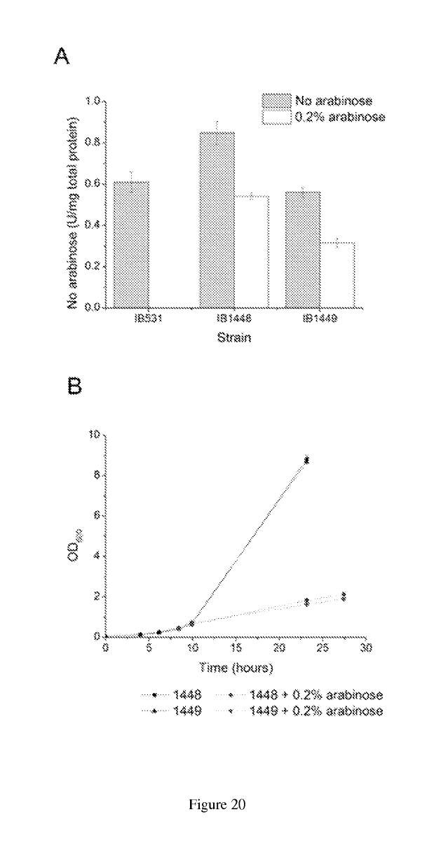

In the absence of arabinose, the newly constructed strains, IB1448 and IB1449, had growth rates similar to the previously constructed strain IB1863, with expression of SspB controlled by the tet promoter. Pfk activity was dependent upon arabinose addition, as expected; however, even in the presence of excess glucose, the arabinose addition still resulted in a decline in Pfk activity after one hour (FIG. 20A) and eventual growth arrest (FIG. 20B).

### Acyl-Homoserine Lactone-Inducible Knockdown of Pfk Activity

A system based on bacterial quorum sensing was developed to allow autonomous switching based on acyl-homoserine lactone (AHL) buildup during biomass formation without requiring nutrient depletion. The system selected for testing was based on the esa QS system from P. stewartii. The two known promoter architectures for this system (EsaR activation, EsaR repression), allow two different configurations of the system to be tested: post-translational control and transcriptional control. The post-translational control system functions by the same principles described for the PphoA and PBAD systems, with SspB expression controlled by an inducible promoter. In this case, the PesaR promoter was used to drive SspB expression, resulting in the induction of SspB upon buildup of 3OC6HSL. The presence of a characterized promoter, PesaS, with the opposite response (e.g., activated only in the absence of 3OC6HSL) also allowed for testing of the direct transcriptional control of Pfk-I. The SsrA degradation tag was still appended to the coding sequence of pfkA to facilitate rapid depletion of the enzyme after cessation of transcription. The potential complication of transcriptional control is that it could require re-tuning of Pfk expression levels, as the strength of the PesaS promoter was not likely to match that of the native pfkA promoter.

There are two main points for controlling the timing of the switch from high Pfk activity (growth) to low Pfk activity (production). The first is through control of expression level of the AHL synthase EsaI, which directly controls the rate of 3OC6HSL accumulation in the system. The other control point comes through utilization of EsaR variants, which have been evolved for increased or reduced sensitivity to 3OC6HSL and show changes in the binding affinity to this molecule (Shong et al., 2013). Thus, with the same rate of 3OC6HSL accumulation, different EsaR variants would be expected to show different binding responses and different times of promoter derepression or deactivation.

Post-Translational Control through Use of P_{EsaR} to Control SspB Expression

Strains were constructed initially based on the PesaR-SspB system, as it was most similar to the autonomous systems previously tested and therefore did not require additional tuning of Pfk expression, since the previously characterized constitutive promoters for pfkA were used. The preliminary strains constructed in this manner, IB2250 and IB2265, were incapable of growth on glucose minimal medium. Pfk activity levels were measured after growth on LB and found to be very low, indicating the leaky expression of SspB was likely resulting in the low Pfk-I levels, even without induction by addition of 3OC6HSL. An RBS library for PEsaR-sspB was constructed as described in Materials and Methods below. Several colonies were isolated which showed growth on glucose minimal medium in the absence of 3OC6HSL and a strong reduction in growth upon 3OC6HSL addition, as expected. Several of the functional strains were sequenced, and the sequences were also analyzed using the RBS calculator (Salis, 2011). All of the functional strains sequenced showed a lower predicted translation initiation rate than the parent strain, indicating that the reduction in baseline expression of SspB likely restored the desired behavior of the strains.

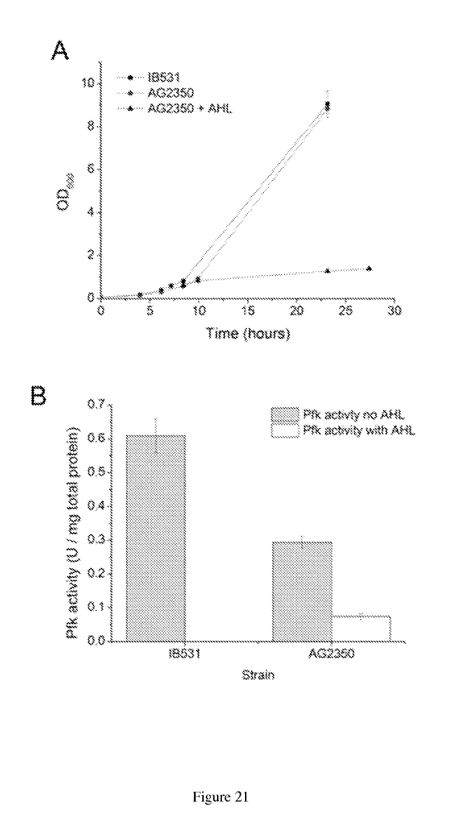

Two of the isolated strains, AG2349 and AG2350, were retained for further testing and development, and the RBS sequences for these strains can be found in Table 4. FIGS. 21A and 216B illustrate the response in growth and Pfk activity, respectively, to the exogenous addition of 3OC6HSL in AG2350. As expected, a decline in Pfk activity was observed, along with growth arrest. AG2350 still showed a lowered baseline Pfk activity when compared to the wild-type control IB531, but its growth on glucose was not significantly impaired.

Transcriptional Control through Use of P_{easS} to Control Pfk-I

In conjunction with the development of post-translational control based on SspB expression, direct transcriptional control of Pfk-I expression via the PesaS promoter was also explored. IB646 and IB2275 were initially constructed to test the potential of this strategy. The two strains both contained degradation-tagged pfkA drive by the PesaS promoter, but each utilized a different variant of EsaR for promoter activation, integrated as described in Materials and Methods below. IB646 contained an expression cassette for wild-type EsaR, while IB2275 expressed the variant EsaRI170V, which shows a reduced binding affinity for 3OC6HSL (Shong et al., 2013).

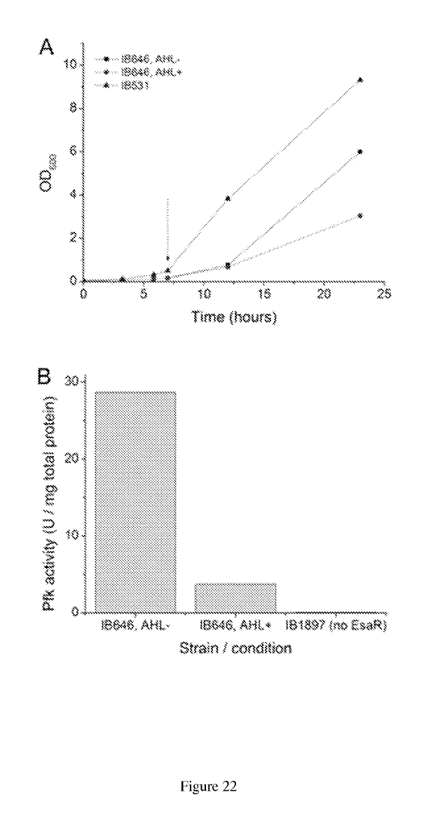

As expected, the Pfk-I expression levels in both strains were poorly matched to the wild-type levels. Pfk activity was shown to be up to 40 times greater than the wild-type levels. Growth was also impaired on glucose minimal medium and final biomass levels were substantially lower than those observed in the wild-type; however, growth arrest was still be observed upon exogenous 3OC6HSL addition, indicating control over Pfk activity was likely being achieved (FIG. 22A). Preliminary tests of IB646 indicated a large drop in Pfk activity occurred upon the addition of 3OC6HSL, with the activity falling below wild-type levels (FIG. 22B). Additional testing with expression of the AHL synthase EsaI (described in more detail in below) confirmed very strong control of Pfk activity in both IB646 and IB2275, and activity repression to below wild-type levels.

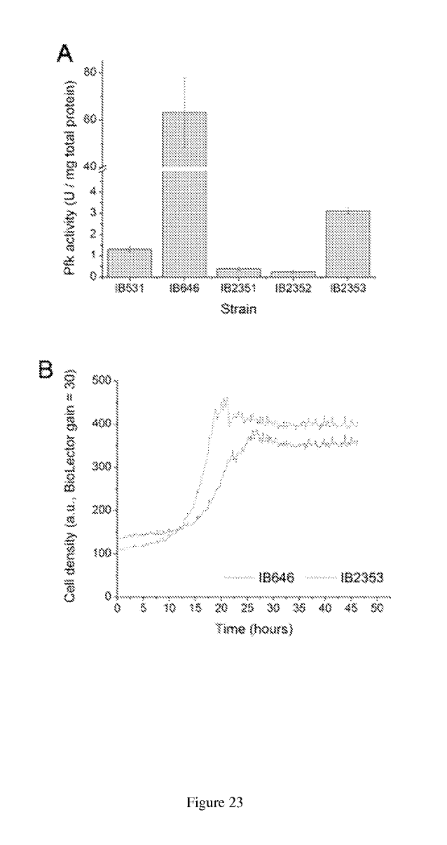

The reduction in final OD due to high Pfk activity in IB646 and IB2275 was undesirable. As the initial promoter construct utilized a strong RBS, an RBS library was constructed for IB646 to reduce baseline Pfk activity via the protocol outlined in below. Several colonies were screened from the library. Some exhibited no growth on glucose minimal medium even in the absence of 3OC6HSL, but two colonies were isolated that were responsive to 3OC6HSL and showed improved growth on glucose minimal medium relative to IB646. Sequencing of colony PCR products and analysis of the predicted RBS strength via the RBS calculator (Salis, 2011) revealed that the colonies that did not grow on glucose had predicted translation initiation rates 20-50 times lower than the original RBS. The two colonies showing improved growth both had the same RBS, with a predicted translation initiation rate of 3096, 7.3 times lower than the original RBS. Two of the strains incapable of growth on glucose, IB2351 and IB2352, were selected for further analysis, along with the improved strain, IB2353. Pfk activity was measured in these three strains during growth on M9+0.4% glycerol and found to be consistent with the observed phenotype. IB2351 and IB2352 showed activity lower than the wild-type control, IB531, while IB2353 showed intermediate activity between the wild-type and the parent, IB646 (FIG. 23A). While IB2353 did not show the complete recovery of wild-type growth and final OD, it did show improvement over the parent strain IB646 (FIG. 23B).

### Linking to EsaI Expression for Autonomous Control

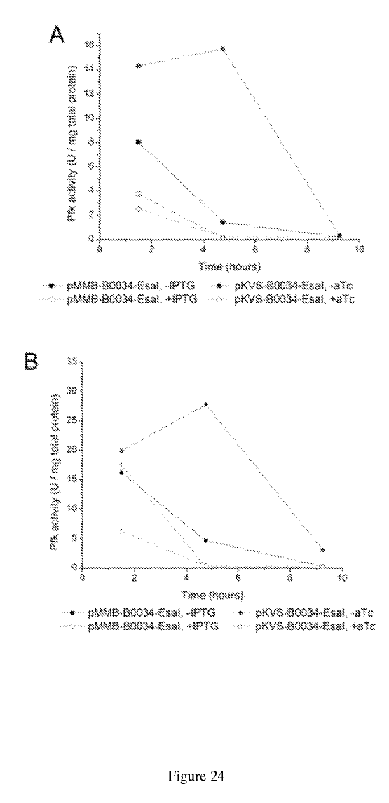

In order for either of these systems to function autonomously in a fermentation, without exogenous AHL addition, the expression of EsaI was also required. The expression level of EsaI was expected to affect the rate of 3OC6HSL synthesis, with lower expression levels expected to result in slow 3OC6HSL accumulation, resulting in the knockdown of Pfk-I expression later in the fermentation, when more biomass is present. EsaI was expressed initially from a plasmid in strains IB646 (FIG. 24A) and IB2275 (FIG. 24B). The medium copy plasmid, pKVS-B0034-EsaI, has very tight control of expression through use of the tet promoter. Due to the leaky expression of EsaI, the Pfk activity switched to the “off” state much later than if EsaI was expressed at high levels from inoculation. A similar trend was seen for the expression from the plasmid pMMB-B0034-EsaI; however, as the lac promoter has higher leaky expression, there is a smaller difference in the time of switching. It is also clear that there is a difference in behavior between the strain with wild-type EsaR (IB646) and the strain with the less sensitive EsaR variant, EsaRI170V (IB2275). With the less sensitive EsaR variant, higher Pfk activity levels were reached before AHL buildup can effect a decline in pfkA expression. The time at which Pfk activity falls below the critical threshold for a given expression level of EsaI is also altered, providing another route for modulation through switching time.

Control through the plasmid-based expression of EsaI requires both an additional antibiotic selection as well as precise inducer addition. To make the system autonomous, a variety of constitutive EsaI expression cassettes were integrated into the genome, generating a series of strains that automatically turned off Pfk-I expression at the desired cell density, without any outside intervention. As described in below, an existing promoter and 5′ UTR library was used to guide the design of EsaI expression cassettes of different strengths (Mutalik et al., 2013). These EsaI expression cassettes were integrated both into strains utilizing direct transcriptional control of pfkA (IB6464, IB2275), as well as a strain relying on the controlled degradation of Pfk-I through SspB expression (AG2349). The promoter and 5′ UTR sequences integrated into each strain are listed in Table 4 with the strains containing each integrated cassette denoted by the parent strain number followed by the promoter/RBS pair designation (e.g. IB646+L18).

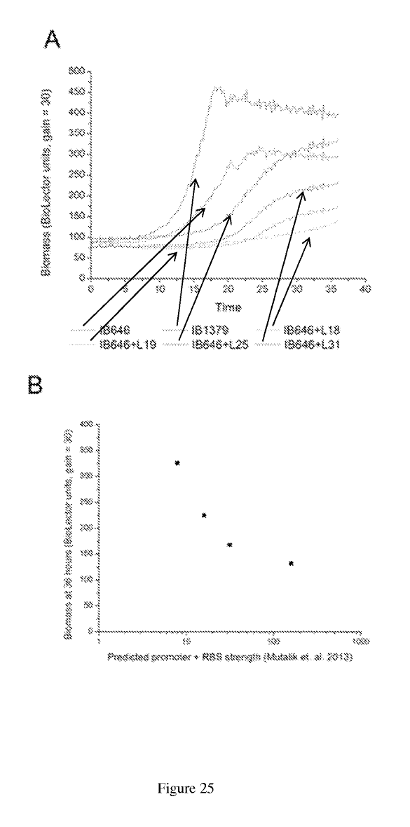

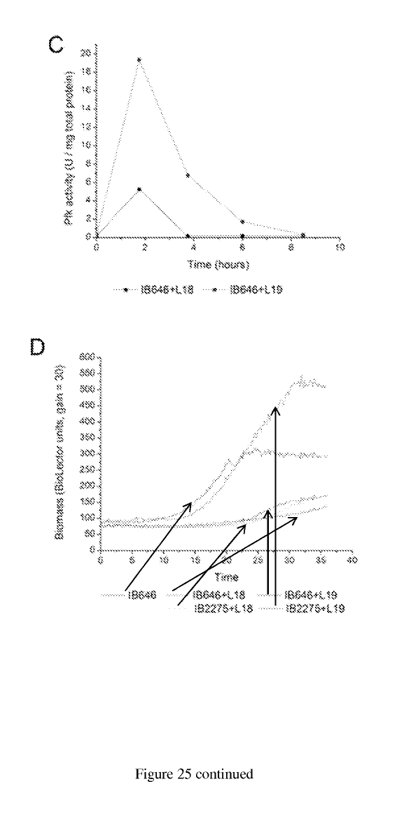

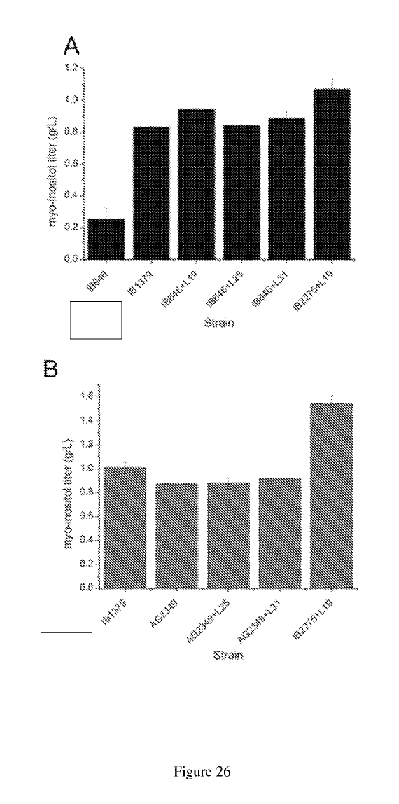

The varying EsaI expression levels resulted in strains with a variety of growth profiles in glucose minimal medium (FIG. 25A). The OD of the cultures at 36 hours corresponded inversely with the predicted strength of the promoter/5′ UTR combination (FIG. 250B). Time course activity measurements in strain IB646+L18 and IB646+L19 in LB showed that Pfk activity declined over the course of the culture (FIG. 25C). The time at which Pfk activity fell below wild-type levels also followed the trend; switch occurring around 4 hours for the stronger L18 expression cassette and around 8 hours for the weaker L19 cassette.

Switching time could also be altered by integrating the EsaI expression cassettes into IB2275. Using the same expression cassette, the arrest of growth was delayed in the IB2275 background relative to the IB646 background (FIG. 25D), as it takes longer to build up a sufficient amount of 3OC6HSL to bind to EsaRI170V.

Several EsaI expression cassettes were also integrated into AG2349, which relies on the expression of SspB from PesaR to increase the rate of Pfk-I degradation and reduce the Pfk activity. In those strains, a long lag time was observed in glucose minimal medium after inoculating working cultures from a starter culture. This lag is likely related to the need to dilute out existing SspB in the cells from the starter before Pfk-I can build up and growth can resume. In contrast, with direct transcriptional control of pfkA, production of the enzyme can immediately resume upon dilution of 3OC6HSL by inoculation into fresh medium. The addition of 0.2% casamino acid to the medium when growing strains based on AG2349 improved their growth behavior, allowing several divisions to occur independent of glucose utilization. Once growth did occur, the rate was very similar to that of IB1379.

Through the varied constitutive expression of EsaI, a set of strains have been developed for which switching from “growth mode” (high Pfk activity) to “production mode” (low Pfk activity) is completely autonomous, requiring no intervention via inducer addition to the medium or limitation of oxygen supply. These strains can be used for production of myo-inositol, glucaric acid, or other metabolites derived from G6P and F6P. The creation of a series of strains allows the optimal strain to be selected for each application, as later switching may be more desirable for cases where intensive growth and protein expression is initially required. The strains can be used to explore trade-offs between yield and titers, allowing the point of maximum productivity to be achieved.

### Myo-inositol Production in Autonomous Strains

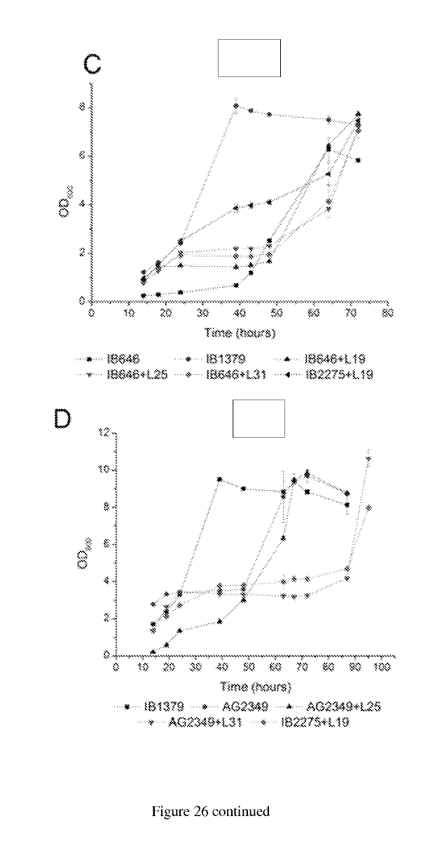

Given the success in achieving autonomous control of growth utilizing components of the esa quorum sensing system, several of the strains were transformed with pTrc-INO1 and tested to determine if the switching could successfully be used to increase myo-inostiol (MI) production. Strains based on IB646 and IB2275 were tested in modified MOPS minimal medium+10 g/L glucose, while strains based on AG2349 were tested in the same medium supplemented with an additional 0.2% casamino acid. The results for both conditions are shown in FIGS. 26A and 26B.

MI production was very poor in IB6464, given its poor growth profile and high Pfk activity. However, through EsaI expression and control of Pfk activity, MI production could be recovered back to levels at or above that in IB13794 with native pfkA expression. Strain IB2275+L19-I showed the greatest improvement over IB13794, with 30% higher titers. This was validated in a second run, and also in casamino acid-supplemented medium (FIG. 25B), where a 43% improvement in titers was achieved. The growth profiles also showed the expected shape, with a clear period of growth arrest (FIG. 25C). Although the parent strain of AG2349 performed better than IB646 under plasmid-free conditions, its growth impaired more severely by pTrc-INO1 expression than expected, given the close match to IB1379 under plasmid-free conditions (FIG. 26D). Activity profiles (data not shown) showed a decline in AG2349-I activity over the course of the fermentation in the absence of EsaI, likely due to leaky SspB expression, and decreased activity of the BioFab promoter in the stationary phase.

### Discussion

The SspB expression module can be adapted for multiple modes of autonomous control, including nutrient starvation and quorum sensing. Leaky baseline expression of SspB can be tuned by modulation of the RBS sequence, allowing a variety of promoter systems with differing baseline expression and induction behaviors to be utilized. In addition to control through a single inducible promoter, the expression of SspB was also coupled with multi-input cellular logic functions, such as AND gates (Anderson et al., 2007), allowing multiple factors to be used in determining switching behavior.

While the control strategies based on the phoA and PBAD promoters both functioned with respect to the induction of SspB and modulation of Pfk activity, they did not appear to be feasible strategies for generating improvements in myo-inositol production. Careful selection of promoter systems is required with respect to external conditions, as well as changes in cell physiology associated with the induction signal. Generally, use of nutrient starvation promoters may suffer from the inability to control the associated effect of broad changes in cell physiology. Use of targeted signals (e.g., buildup of a pathway intermediate) or non-native signals may provide a more robust basis for the manipulation of individual nodes in metabolism.

The quorum sensing-based strategy offers one example of utilization of a non-native signal, 3OC6HSL. The rate of accumulation of this molecule can be artificially controlled through varying the expression of the cognate AHL synthase or use of transcription factor variants with differing affinities for AHL. Build-up of this non-native signal was effectively used to trigger SspB expression in an autonomous manner, dependent only on the properties of the cell itself. This offers a powerful strategy for controlling the period of biomass accumulation, independent of medium formulation, inducer addition, and process conditions. As a result, cells can be continuously maintained in medium and process conditions favoring rapid growth and high accumulation of heterologous pathway enzymes, but after a period of growth, they will still switch physiological states to one more favorable to production of a desired small molecule, and this will occur at a point predetermined by the genetics of the strain utilized.

The esa quorum sensing system also offered an opportunity to explore the direct transcriptional control of pfkA expression, as well as Pfk-I degradation through SspB expression. The PesaS promoter differs from typically utilized quorum sensing promoters like Plas and Plux, in that its default state is “on” in the absence of AHL, allowing accumulation of 3OC6HSL to be used to turn off expression of the target gene (Schu et al., 2009). Both this system and the PesaR-sspB expression system could be coupled with expression of the AHL synthase EsaI to result in the autonomous knockdown of Pfk activity and growth arrest at relevant cell densities. One limitation of the SspB-based system for autonomous control is that, if SspB becomes induced during the starter culture, it must be diluted out through several doublings in the working culture before normal growth can resume. In contrast, with transcriptional control, Pfk activity can recover more quickly upon transfer from starter to working culture because expression of the enzyme starts immediately upon inoculation in fresh medium without 3OC6HSL.

The quorum sensing based strategy can be utilized to achieve increases in production of a heterologous product, myo-inositol. Increases in MI titer up to 30% were achieved on glucose as a sole carbon source when compared to a strain with native pfkA expression in a 72 hour batch, without any intervention or addition of inducer. Further optimization of Pfk-I levels in the autonomous strains could result in larger increases in yield and titer.

### Conclusions

Manual timing of Pfk knockdown resulted in maximum two-fold improvement in yields and titers (Brockman and Prather, 2015), and a similar improvement would likely result from an optimized quorum sensing system. Additionally, as a series of strains have been developed with growth arrest at different cell densities, it should be possible to find trends in yield and titer, with increasing yield for earlier growth arrest.

Additional points of control may be added; for example, expression of glucose-6-phosphate dehydrogenase could be actively controlled, rather than relying on a gene knockout, or expression of INO1 could be delayed, to reduce expression stress early in the fermentation. Differences in timing between the expressions of different genes could potentially be achieved by utilizing previously developed variants of the PesaR promoter with varied induction profiles in response to AHL accumulation (Shong and Collins, 2013) or by utilizing secondary signals.

### Materials and Methods for Example 3

### Strains and Plasmids

E. coli strains and plasmids used in this study are listed in Table 3. E. coli strain DH10B was used for molecular cloning and plasmid preparation. Strains IB531 and IB1643 were described in earlier (Brockman and Prather, 2015). Strain IB1624 was constructed using the previously described method for construction of IB1643. The pfkA cassette used for replacement of the native locus was identical to that used in IB1643, except that the constitutive promoter used to drive pfkA expression in IB1643 (apFAB114) was replaced with a different promoter from the BIOFAB library, apFAB104, which has a higher promoter strength (BIOFAB, 2012).

Construction of P_{phoA}, P_{BAD}, and P_{EsaR}-Based SspB Expression Cassettes

For construction of the P_{phoA}-sspB expression cassette, the phoA promoter and a synthetic 5′ UTR were appended to sspB using an extended 5′ overhang on the PCR primer for amplification of the sspB sequence from the E. coli genome. The AraC-P_{BAD}-sspB cassette was generated by cloning of the sspB sequence, preceded by RBS B0034 (partsregistry.org), into pBAD30 between the EcoRI and BamHI restriction sites, followed by amplification of the entire arabinose-inducible expression cassette by PCR. The EsaR-P_{EsaR}-sspB cassette was originally constructed using overlap extension PCR and cloned into the plasmid pSB3K3 to yield pSB3K3-EsaR-P_{esaR}-sspB. The EsaR coding sequence was amplified from pAC-EsaR (Shong et al., 2013) and the constitutive promoter apFAB104 was appended to drive EsaR expression. The P_{EsaR} sequence was amplified from pCS-P_{esaR}-lux (Shong et al., 2013). The entire EsaR-P_{EsaR}-SspB expression cassette was then PCR amplified from pSB3K3-EsaR-P_{esaR}-sspB for use in integration steps. Restriction sites were also appended as needed during PCR amplification of all cassettes to allow for ligation into the integration vector. Integration of the SspB expression cassettes into the genome was carried out via “clonetegration” (St-Pierre et al., 2013). The pOSIP-CH backbone and the desired SspB expression cassettes were digested with KpnI and Pstl and ligated overnight. The ligation product was used to transform strains IB1624 and IB1643 for integration at the HK022 locus. The phage integration genes and antibiotic resistance cassette were cured with pE-FLP, yielding the corresponding strains listed in Table 3.

### Construction of RBS Library for P_{esaR}-SspB

To adjust the expression level of SspB from the P_{esaR} promoter and reduce leaky expression, an RBS library was constructed using strain IB2265 as a basis. The original 5′ UTR consisted of the following sequence: “caattcattaaagaggagaaaggatcc” (SEQ ID NO: 4) The start of the SspB coding sequence contains a protospacer adjacent motif (PAM, underlined) “atggattgt”, allowing a Cas9-based targeting method to be used for construction of the RBS library. Briefly, the sgRNA targeting the 20 bp upstream from the PAM, which included the RBS sequence, was inserted into plasmid pKD-sgRNA-sspBRBS via circular polymerase extension cloning. Strain IB2265 was transformed with this plasmid, along with plasmid pCas9-CR4 for inducible expression of Cas9. An 80 bp oligonucleotide with two degenerate nucleotides in the RBS and an additional degenerate nucleotide adjacent to the PAM was then transformed into the strain, and cell survival after Cas9 cleavage of the genome was used to select for cells that integrated the genomic changes encoded in the oligonucleotide. Surviving colonies were screened for growth on glucose minimal medium, and several were isolated with restored growth. Among the successful colonies tested, two were saved for further characterization and testing. The first contained 5′ UTR sequence “caattcattaaagagctgaaaggatca” (SEQ ID NO: 5) (base changes underlined) and was designated AG2349. The second contained the 5′ UTR sequence “caattcattaaagagtcgaaaggatca” (SEQ ID NO: 6) (base changes underlined) and was designated AG2350.

### Construction of Pₑₐₛₐs-pfkA Variants

The replacement of the native pfkA locus with P_{EsaS}-pfkA(degradation tagged) was carried out via a “landing pad” method (Kuhlman and Cox, 2010). Overlap extension PCR was used to append the P_{EsaS} promoter, amplified from pCS P_{EsaS}-lux, to the 5′ UTR from the BIOFAB library and the pfkA coding sequence. Either the native SsrA tag (AADENYALAA, “LAA”) or a modified version (AADENYSENYADAS (SEQ ID NO: 3), “DAS+4”) was also appended to the pfkA coding sequence using primer overhangs. The products were cloned into the vector pTKIP-neo by restriction digest with HindIII and KpnI, yielding pTKIP-P_{esaS}-pfkA(LAA) and pTKIP-P_{esaS}-pfkA(DAS+4). Lambda-red mediated recombination was used to introduce the tetracycline resistance marker and “landing pad” sequences amplified from pTKS/CS into the genome at the pfkA locus. The resultant strain was then transformed with pTKRED and either pTKIP-P_{esaS}-pfkA(LAA) and pTKIP-P_{esaS}-pfkA(DAS+4). Integration of the construct from the pTKIP plasmid into the genome was achieved as described previously (Kuhlman and Cox, 2010). The kanamycin resistance cassette remaining after integration was cured by expression of FLP recombinase from pCP20 to yield strains IB1897 and IB898. For activation of the P_{EsaS} promoter, EsaR is required. A cassette containing the apFAB104 promoter driving expression of EsaR was integrated into the genome using the “clonetegration” method described in Section 4.2.2. Additionally, an alternative expression cassette with previously described EsaR variant, EsaRI170V, that is less sensitive to its cognate autoinducer (Shong et al., 2013), was prepared. IB1897 and IB1898 were both separately transformed with ligation mixtures containing the clonetegration plasmid pOSIP-CH and either the EsaR and EsaRI170V cassette. The successful integration events were integration of EsaR into IB1897 and integration of EsaRI170V into IB1898. Those strains were designated IB646 and IB2275, respectively.

### Construction of an RBS Library for Pₑₐₛₐs-pfkA

The expression level of Pfk-I from the intial P_{esaS} promoter-RBS construct was much higher than the wild type expression of Pfk-I. To adjust expression of Pfk-I, an RBS library was constructed in strain IB646 using the same Cas9 counterselection method described above for adjustment of SspB expression from the P_{esaR} promoter. The useable PAM sequence for Cas9 recognition was contained within the coding sequence of pfkA. For selection, two silent mutations were made to the coding sequence of pfkA, and then an additional two degenerate nucleotides were included in the RBS sequence to create the RBS library. The library members were screened for improved growth in minimal medium relative to the parent strain. One strain was isolated with improved growth and the RBS sequence “caattcattaaagaggcgaaaggatcc” (SEQ ID NO: 7) (base change underlined). This strain was designated IB2353. Two strains that did not show growth on glucose minimal medium were also retained for analysis,

| | |
|---|---|
| IB2351 | |
| (“caattcattaaagagttgaaaggatcc” | (SEQ ID NO: 8)) |
| and | |
| IB2352 | |
| (“caattcattaaagagtggaaaggatcc”. | (SEQ ID NO: 9)) |

### Integration of EsaI Expression Cassettes

Plasmid-based constructs for EsaI expression were constructed by PCR amplification of the esaI coding sequence from pAC-EsaR-EsaI (Shong and Collins, 2013) with addition of RBS B0034 on the primer, and cloning into the pKVS45 or pMMB206 backbones at the EcoRI and HindIII restriction sites.

For genomic integration of EsaI constructs with varying expression levels, a series of EsaI expression cassettes were generated via PCR. The promoter and 5′ UTR combinations for these cassettes were taken from those evaluated by Mutalik et. al. to give a range of expression levels (Mutalik et al., 2013). The EsaI expression cassettes with digested with KpnI and SphI, as was the clonetegration vector pOSIP-KO. The target strain was then transformed with the ligation mixture and screened via the standard procedure for one step cloning and integration (St-Pierre et al., 2013). Use of the pOSIP-KO vector can result in integration at one of two 186 attachment sites. Strains were screened for integration at the primary site, and only those with primary site integration were used. Table 4 lists the set of promoter and 5′ UTR sequences that resulted in moderate accumulation of 3OC6HSL and were appropriate for further testing. These were integrated into the strain backgrounds IB646, IB2275, and AG2349, and the resulting strains are denoted by their parent strain number and the EsaI expression cassette notation (e.g. IB646-L18).

During all integration steps, colonies were screened via colony PCR with OneTaq master mix. PCR amplifications for cloning or genomic integration were carried out with Q5 polymerase. Enzymes utilized for PCR amplification, restriction digests, and ligation were obtained from New England Biolabs (Ipswich, Mass.). Oligonucleotides were obtained from Sigma-Genosys (St. Louis, Mo.) or Integrated DNA Technologies (Coralville, Iowa).

### Culture Medium and Conditions

For plasmid preparation and genetic manipulations, strains were cultured in Luria-Bertani (LB) medium at either 30° or 37° C. Temperature sensitive plasmids were cured at 42° C. Except where noted, growth and production experiments were carried out at 30° C. in a modified MOPS medium containing 10 g/L D-glucose, 3 g/L NH₄Cl, 2 mM MgSO₄, 0.1 mM CaCl₂, 40 mM MOPS, 4 mM tricine, 50 mM NaCl, 100 mM Bis-Tris, 134 μM EDTA, 31 μM FeCl₃, 6.2 μM ZnCl₃, 0.76 μM CuCl₂, 0.42 μM CoCl₂, 1.62 μM H₃BO₃, and 0.081 μM MnCl₂. Dibasic potassium phosphate (K₂HPO₄) was supplemented into this medium at 1 g/L, except under phosphate limitation conditions, where it was added at the concentrations specified in the above. For strains containing pTrc-INO1, carbenicillin (100 μg/mL) was added for plasmid maintenance. Seed cultures were initiated using a 1:100-1:500 dilution from LB cultures and were grown at 30° C. for 18-24 hours in modified MOPS with 1 g/L K₂HPO₄, until mid-exponential phase was reached. The seed cultures were then used for inoculation of working cultures for growth, activity, and production experiments.

For myo-inositol production experiments, working cultures of 50 ml in 250 ml baffled shake flasks were inoculated to OD=0.05 from seed cultures, and 50 μM β-D-1-thiogalactopyranoside (IPTG) was added at inoculation. Flasks were incubated at 30° C. with 250 rpm shaking and 80% humidity. Samples were taken periodically for measurement of enzyme activity, protein levels, and extracellular metabolites. For growth screening experiments during RBS library construction, working cultures were inoculated into in 48-well flower plates for OD monitoring in a BioLector (m2p labs, Baseweiler, Germany) and incubated at 30° C., 1200 rpm, and 80% humidity.

### Phosphofructokinase Activity Assays

Enzymatic activity assays were carried out on crude lysates. For preparation of lysates, samples of 5-10 ml of cell culture were collected, frozen at −80° C., and then resuspended in 50 mM Tris-HCl, pH 7.4 (0.25-1 ml, depending on cell density). Cells were lysed via bead beating for 5 minutes and lysates clarified by centrifugation at 15,000×g for 15 minutes. Phosphofructokinase activity was assayed using a protocol adapted from Kotlarz and Buc (Kotlarz and Buc, 1982; Kotlarz et al., 1975). The assay mixture consisted of 0.1 M Tris-HCl (pH 8.2), 10 mM MgCl2, 1 mM ATP, 0.2 mM β-NADH, 1 mM fructose-6-phosphate (F6P), 1 mM NH4Cl, 0.01% Triton X-100, 0.83 U aldolase, 0.42 U triosephosphate isomerase, and 0.42 U glycerophosphate dehydrogenase. Reaction progress was followed by measurement of absorbance at 340 nm. One unit of Pfk activity was defined as the amount required to convert 1.0 μmole of ATP and D-fructose 6-phosphate to ADP and fructose 1,6-bisphosphate per minute at pH 8.2 and room temperature.

### Measurement of Myo-Inositol Titers and Yields

Glucose and myo-inositol levels were quantified by high performance liquid chromatography (HPLC) on an Agilent 1100 or 1200 series instrument (Santa Clara, Calif.) with an Aminex HPX-87H anion exchange column (300 mm by 7.8 mm; Bio-Rad Laboratories, Hercules, Calif.). Sulfuric acid (5 mM) at a flow rate of 0.6 mL/min was used as the mobile phase. Compounds were quantified from 10 μL sample injections using a refractive index detector. Column and refractive index detector temperatures were held at 55° C.

### Tables of Example 3

TABLE 3
Strains and Plasmids

| Strain/plasmid | Genotype | Reference/source |
|---|---|---|
| DH10B | F⁻ mcrA Δ(mrr-hsdRMS-mcrBC) φ80lacZΔM15 ΔlacX74 recA1 | Life Technologies |
| | endA1 araD139 Δ(ara, leu)7697 galU galK λ-rpsL nupG | (Carlsbad, CA) |

MG1655 parent strains

| | | |
|---|---|---|
| IB531 | MG1655 ΔendA | Prather Lab |
| IB1379 | MG1655 ΔendA Δzwf ΔpfkB | (Brockman and |
| | | Prather, 2015) |

Biofab promoter replacments for pfkA

| | | |
|---|---|---|
| IB1643 | MG1655 ΔendA Δzwf ΔpfkB ΔsspB pfkA::114-pfkA(DAS + 4) | (Brockman and |
| | | Prather, 2015) |
| IB1624 | MG1655 ΔendA Δzwf ΔpfkB ΔsspB pfkA::104-pfkA(DAS + 4) | This study |

Pₜₑₜ-sspB integration

| | | |
|---|---|---|
| IB1863 | MG1655 ΔendA Δzwf ΔpfkB ΔsspB pfkA::114-pfkA(DAS + 4) | (Brockman and |
| | HK022::TetR-P_{LtetO}-sspB | Prather, 2015) |

P_{phoA}-sspB integrations

| | | |
|---|---|---|
| IB643 | MG1655 ΔendA Δzwf ΔpfkB ΔsspB pfkA::114-pfkA(DAS + 4) | This study |
| | HK022::P_{phoA}-sspB | |
| IB1509 | MG1655 ΔendA Δzwf ΔpfkB ΔsspB pfkA::104-pfkA(DAS + 4) | This study |
| | HK022::P_{phoA}-sspB | |

P_{BAD}-sspB integrations

| | | |
|---|---|---|
| IB1449 | MG1655 ΔendA Δzwf ΔpfkB ΔsspB pfkA::114-pfkA(DAS + 4) | This study |
| | HK022::AraC-P_{BAD}-sspB | |
| IB1448 | MG1655 ΔendA Δzwf ΔpfkB ΔsspB pfkA::104-pfkA(DAS + 4) | This study |
| | HK022::AraC-P_{BAD}-sspB | |

P_{esaR}-sspB integreations and RBS tuning

| | | |
|---|---|---|
| IB2265 | MG1655 ΔendA Δzwf ΔpfkB ΔsspB pfkA::114- | This study |
| | pfkA(DAS + 4)HK022::EsaR-P_{EsaR}-sspB | |
| IB2250 | MG1655 ΔendA Δzwf ΔpfkB ΔsspB pfkA::104- | This study |
| | pfkA(DAS + 4)HK022::EsaR-P_{EsaR}-sspB | |
| AG2349 | MG1655 ΔendA Δzwf ΔpfkB ΔsspB pfkA::114pfkA(DAS + 4) | This study |
| | HK022::EsaR-P_{EsaR}-RBS157-sspB | |
| AG2350 | MG1655 ΔendA Δzwf ΔpfkB ΔsspB pfkA::114pfkA(DAS + 4) | This study |
| | HK022::EsaR-P_{EsaR}-RBS422-sspB | |
| AG2349 + L25 | MG1655 ΔendA Δzwf ΔpfkB ΔsspB pfkA::114pfkA(DAS + 4) | This study |
| | HK022::EsaR-P_{EsaR}-RBS157-sspB 186::L25-EsaI | |
| AG2349 + L31 | MG1655 ΔendA Δzwf ΔpfkB ΔsspB pfkA::114pfkA(DAS + 4) | This study |
| | HK022::EsaR-P_{EsaR}-RBS157-sspB 186::L31-EsaI | |

P_{esaS}-pfkA promoter replacements and RBS tuning

| | | |
|---|---|---|
| IB1897 | MG1655 ΔendA Δzwf ΔpfkB pfkA::P_{EsaS}-pfkA(DAS + 4) | This study |
| IB1898 | MG1655 ΔendA Δzwf ΔpfkB pfkA::P_{EsaS}-pfkA(LAA) | This study |
| IB646 | MG1655 ΔendA Δzwf ΔpfkB pfkA::P_{EsaS}-pfkA(DAS + 4) | This study |
| | HK022::104-EsaR | |
| IB2275 | MG1655 ΔendA Δzwf ΔpfkB pfkA::P_{EsaS}-pfkA(LAA) | This study |
| | HK022::104-EsaRI170V | |
| IB2351 | MG1655 ΔendA Δzwf ΔpfkB pfkA::P_{EsaS}-RBS1087- | This study |
| | pfkA(LAA) HK022::104-EsaRI170V | |
| IB2352 | MG1655 ΔendA Δzwf ΔpfkB pfkA::P_{EsaS}-RBS468-pfkA(LAA) | This study |
| | HK022::104-EsaRI170V | |
| IB2353 | MG1655 ΔendA Δzwf ΔpfkB pfkA::P_{EsaS}-RBS3096- | This study |
| | pfkA(LAA) HK022::104-EsaRI170V | |
| IB646 + L18 | MG1655 ΔendA Δzwf ΔpfkB pfkA::P_{EsaS}-pfkA(DAS + 4) | This study |
| | HK022::104-EsaR 186::L18-EsaI | |
| IB646 + L19 | MG1655 ΔendA Δzwf ΔpfkB pfkA::P_{EsaS}-pfkA(DAS + 4) | This study |
| | HK022::104-EsaR 186::L19-EsaI | |
| IB646 + L25 | MG1655 ΔendA Δzwf ΔpfkB pfkA::P_{EsaS}-pfkA(DAS + 4) | This study |
| | HK022::104-EsaR 186::L25-EsaI | |
| IB646 + L31 | MG1655 ΔendA Δzwf ΔpfkB pfkA::P_{EsaS}-pfkA(DAS + 4) | This study |
| | HK022::104-EsaR 186::L31-EsaI | |
| IB2275 + L18 | MG1655 ΔendA Δzwf ΔpfkB pfkA::P_{EsaS}-pfkA(LAA) | This study |
| | HK022::104-EsaRI170V 186::L18-EsaI | |
| IB2275 + L19 | MG1655 ΔendA Δzwf ΔpfkB pfkA::P_{EsaS}-pfkA(LAA) | This study |
| | HK022::104-EsaRI170V 186::L19-EsaI | |

Strains transformed with pTrc-INO1 are denoted by-I

| | | |
|---|---|---|
| Plasmids | | |
| pAC-EsaR | pACYC184 with lac promoter, esaR gene, and | (Shong et al., 2013) |
| | transcriptional terminator | |
| pBAD30 | p15A, Amp^{R}, araC, P_{BAD} | (Guzman et al., |
| | | 1995) |
| pBAD-SspB | p15A, Amp^{R}, araC, P_{BAD}-RBS B0034-sspB | This study |
| pCas9-CR4 | Pₜₑₜ-Cas9 (S. pyogenes), Spec^{R} | (Reisch and |
| | | Prather, in |
| | | preparation) |
| pCP20 | Rep^{a}, Amp^{R}, Cm^{R}, FLP recombinase expressed by λ pᵣ under | CGSC #7629 |
| | control of λ cI857 | |
| pCS-P_{EsaR}-lux | pCS26 with P_{esaR} promoter controlling expression of | (Shong et al., 2013) |
| | luxCDABE | |
| pCS-P_{EsaS}-lux | pCS26 with P_{esaS} promoter controlling expression of | (Shong et al., 2013) |
| | luxCDABE | |
| pE-FLP | oriR101, repA101ts, Amp^{R}, FLP recombinase expressed by | (St-Pierre et al., |
| | pE | 2013), Addgene |
| | | Plasmid #45978 |
| pKD-sgRNA- | oriR101, repA101ts, Amp^{R}, araC, araBp-λ_{γ}-λ_{β}-λₑₓₒ, sgRNA | (Reisch and |
| pfkARBS | targeting pfkA | Prather, in |
| | | preparation) |
| pKD-sgRNA- | oriR101, repA101ts, Amp^{R}, araC, araBp-λ_{γ}-λ_{β}-λₑₓₒ, sgRNA | |
| sspBRBS | targeting RBS for sspB | |
| pKVS45 | p15A, Amp^{R}, tetR, P_{Tet} | (Solomon et al., |
| | | 2012b) |
| pKVS-B0034-EsaI | pKVS45 with RBS B0034-EsaI | This study |
| pMMB206 | IncQ, Cm^{R}, lacI, P_{tac} | (Morales et al., |
| | | 1991) |
| pMMB-B0034-EsaI | pMMB206 with RBS B0034-EsaI | This study |
| pOSIP-CH | pUC ori, RK6γ ori, Cm^{R}, attP HK022, ccdB, HK022 | (St-Pierre et al., |
| | integrase expressed by λ pᵣ under control of λ cI857 | 2013), Addgene |
| | | Plasmid #45980 |
| pSB3K3 | p15A, Kan^{R} | (Shetty et al., 2008) |
| pSB3K3-EsaR-P_{EsaR}- | pSB3K3 with P_{BIOFAB104}-esaR, P_{esaR}-sspB | This study |
| SspB | | |
| pTrc-INO1 | pTrc99A with S. cerevisiae INO1 inserted at the EcoRI and | (Moon et al., 2009) |
| | HindIII sites | |
| pTKIP-neo | ColE1(pBR322) ori, Amp^{R}, Kan^{R} | (Kuhlman and Cox, |
| | | 2010) |
| pTKS/CS | p15A, Cm^{R}, P_{lacIq} tetA | (Kuhlman and Cox, |
| | | 2010) |
| pTKRED | oriR101, repA101ts, Spec^{R}, araC, P_{lac} λ_{γ} λ₆ λₑₓₒ lacI, P_{araB} I- | (Kuhlman and Cox, |
| | SceI | 2010) |
| pTKIP-P_{EsaS}- | pTKIP-neo with P_{EsaS}-pfkA(DAS + 4) | This study |
| PfkA(DAS + 4) | | |
| pTKIP-P_{EsaS}- | pTKIP-neo with P_{EsaS}-pfkA(LAA) | This study |
| PfkA(LAA) | | |

TABLE 4
Promoter and UTR Sequences for Integrated Expression Cassettes

| | | | | |
|---|---|---|---|---|
| | | | | Inte- |
| | | | Degradation tag, | gration |
| Construct | Promoter | 5′ UTR/RBS | DNA sequence | site |

SspB expression cassettes

| | | | |
|---|---|---|---|
| Pₜₑₜ-sspB | tccctatcagtgatagagattgacatccctatcagtg | ctaggtttctccatacccgttttt | HK022 |
| | atagagatactgagcac (SEQ ID NO: 10) | ttgggctagcgaattcaaaga | |
| | | ggagaaatactag | |
| | | (SEQ ID NO: 20) | |
| P_{phoA}-sspB | ctgtcataaagttgtcacggccgagacttatagtcg | caattcattaaagaggagaag | HK022 |
| | cttt (SEQ ID NO: 11) | gatcc | |
| | | (SEQ ID NO: 21) | |
| P_{BAD}-sspB | ccataagattagcggatcctacctgacgctttttatc | acccgtttttttgggctagegaa | HK022 |
| | gcaactctctactgtttctccat | ttcaaagaggagaaatactag | |
| | (SEQ ID NO: 12) | (SEQ ID NO: 22) | |
| P_{esaR}-sspB | gcagattgagtaaccgtgaatgtttgtacaaatgttt | caattcattaaagaggagaaa | HK022 |
| | caaagatgttactatgagtgtcccggccagcatcac | ggatcc | |
| | tttatattttgtgacgtctggccggacgttttccctagt | (SEQ ID NO: 4) | |
| | gttggctgttttagcgacctggccgtacaggtcagg | | |
| | tttttttttaccgctaaacaactgaagccattgtaacct | | |
| | ctgaatgattcattgtaagttactcttaagtatcatctt | | |
| | gcctgtactatagtgcaggttaagtccacgttaagta | | |
| | aaagaagcagc (SEQ ID NO: 13) | | |
| P_{esaR}- | gcagattgagtaaccgtgaatgtttgtacaaatgttt | caattcattaaagagctgaaag | HK022 |
| RBS157- | caaagatgttactatgagtgtcccggccagcatcac | gatca (SEQ ID NO: 5) | |
| sspB | tttatattttgtgacgtctggccggacgttttccctagt | | |
| (AG2349) | gttggctgttttagcgacctggccgtacaggtcagg | | |
| | tttttttttaccgctaaacaactgaagccattgtaacct | | |
| | ctgaatgattcattgtaagttactcttaagtatcatctt | | |
| | gcctgtactatagtgcaggttaagtccacgttaagta | | |
| | aaagaagcagc (SEQ ID NO: 13) | | |
| P_{esaR}- | gcagattgagtaaccgtgaatgtttgtacaaatgttt | caattcattaaagagtcgaaag | HK022 |
| RBS422- | caaagatgttactatgagtgtcccggccagcatcac | gatca (SEQ ID NO: 6) | |
| sspB | tttatattttgtgacgtctggccggacgttttccctagt | | |
| (AG2350) | gttggctgttttagcgacctggccgtacaggtcagg | | |
| | tttttttttaccgctaaacaactgaagccattgtaacct | | |
| | ctgaatgattcattgtaagttactcttaagtatcatctt | | |
| | gcctgtactatagtgcaggttaagtccacgttaagta | | |
| | aaagaagcagc (SEQ ID NO: 13) | | |

pfkA expression cassettes

| | | | | |
|---|---|---|---|---|
| 114-pfkA | tcgacatcaggaaaatttttctgatacttacagccat | caattcattaaagaggagaaa | Gcggcgaacgatgaa | pfkA |
| (DAS + 4) | (SEQ ID NO: 14) | ggatcc (SEQ ID NO: 4) | aactatagcgaaaactat | native |
| | | | gcggatgcgagc | locus |
| | | | (SEQ ID NO: 25) | |
| 104-pfkA | tcgacataaagtctaacctataggatacttacagccat | caattcattaaagaggagaaa | Gcggcgaacgatgaa | pfkA |
| (DAS + 4) | (SEQ ID NO: 15) | ggatcc (SEQ ID NO: 4) | aactatagcgaaaactat | native |
| | | | gcggatgcgagc | locus |
| | | | (SEQ ID NO: 25) | |
| P_{esaS}-pfkA | gctcacaacagtgtaagcgtatccgttattgtttgatttt | caattcattaaagaggagaaa | Gctgctaacgacgaaa | pfkA |
| (LAA) | caaggaaaaaagaaaacattcaggctccatgctgct | ggatcc (SEQ ID NO: 4) | actacgctctggctgct | native |
| | tcttttacttaacgtggacttaacctgcactatagtaca | | (SEQ ID NO: 26) | locus |
| | ggcaagatgatacttaagagtaacttacaatgaatca | | | |
| | ttcagaggttacaatggcttcagttgtttagc | | | |
| | (SEQ ID NO: 16) | | | |
| P_{esaS}-pfkA | gctcacaacagtgtaagcgtatccgttattgtttgatttt | caattcattaaagaggagaaa | Gcggcgaacgatgaa | pfkA |
| (DAS + 4) | caaggaaaaaagaaaacattcaggctccatgctgct | ggatcc (SEQ ID NO: 4) | aactatagcgaaaactat | native |
| | tcttttacttaacgtggacttaacctgcactatagtaca | | gcggatgcgagc | locus |
| | ggcaagatgatacttaagagtaacttacaatgaatca | | (SEQ ID NO: 25) | |
| | ttcagaggttacaatggcttcagttgtttagc | | | |
| | (SEQ ID NO: 16) | | | |
| P_{esaS}- | gctcacaacagtgtaagcgtatccgttattgtttgatttt | caattcattaaagaggcgaaa | Gcggcgaacgatgaa | pfkA |
| RBS3096- | caaggaaaaaagaaaacattcaggctccatgctgct | ggatcc (SEQ ID NO: 7) | aactatagcgaaaactat | native |
| pfkA | tcttttacttaacgtggacttaacctgcactatagtaca | | gcggatgcgagc | locus |
| (DAS + 4) | ggcaagatgatacttaagagtaacttacaatgaatca | | (SEQ ID NO: 25) | |
| (IB2353) | ttcagaggttacaatggcttcagttgtttagc | | | |
| | (SEQ ID NO: 16) | | | |
| P_{esaS}- | gctcacaacagtgtaagcgtatccgttattgtttgatttt | caattcattaaagagttgaaag | Gcggcgaacgatgaa | pfkA |
| RBS1087- | caaggaaaaaagaaaacattcaggctccatgctgct | gatcc (SEQ ID NO: 8) | aactatagcgaaaactat | native |
| pfkA | tcttttacttaacgtggacttaacctgcactatagtaca | | gcggatgcgagc | locus |
| (DAS + 4) | ggcaagatgatacttaagagtaacttacaatgaatca | | (SEQ ID NO: 25) | |
| (IB2351) | ttcagaggttacaatggcttcagttgtttagc | | | |
| | (SEQ ID NO: 16) | | | |
| P_{esaS}- | gctcacaacagtgtaagcgtatccgttattgtttgatttt | caattcattaaagagtggaaag | Gcggcgaacgatgaa | pfkA |
| RBS468- | caaggaaaaaagaaaacattcaggctccatgctgct | gatcc (SEQ ID NO: 9) | aactatagcgaaaactat | native |
| pfkA | tcttttacttaacgtggacttaacctgcactatagtaca | | gcggatgcgagc | locus |
| (DAS + 4) | ggcaagatgatacttaagagtaacttacaatgaatca | | (SEQ ID NO: 25) | |
| (IB2352) | ttcagaggttacaatggcttcagttgtttagc | | | |
| | (SEQ ID NO: 16) | | | |

EsaR expression cassette

| | | | |
|---|---|---|---|
| 104-esaR | tcgacataaagtctaacctataggatacttacagccat | caattcattaaagaggagaaa | HK022 |
| | (SEQ ID NO: 15) | ggatcc (SEQ ID NO: 4) | |

EsaI expression cassettes

| | | | |
|---|---|---|---|
| L18-esaI | ttcacttttaatcatccggctcgtataatgtgtgga | gggcccaagttcacttaaaaa | P186 |
| (P8/BCD21) | (SEQ ID NO: 17) | ggagatcaacaatgaaagcaa | primary |
| | | ttttcgtactgaaacatcttaatc | |
| | | atgcgagggatggtttcta | |
| | | (SEQ ID NO: 23) | |
| L19-esaI | ttcacttttaatcatccggctcgtataatgtgtgga | gggcccaagttcacttaaaaa | P186 |
| (P8/BCD22) | (SEQ ID NO: 17) | ggagatcaacaatgaaagcaa | primary |
| | | ttttcgtactgaaacatcttaatc | |
| | | atgcctaggaagttttcta | |
| | | (SEQ ID NO: 24) | |
| L25-esaI | ttgcctcttaatcatcggctcgtataatgtgtgga | gggcccaagttcacttaaaaa | P186 |
| (P9/BCD22) | (SEQ ID NO: 18) | ggagatcaacaatgaaagcaa | primary |
| | | ttttcgtactgaaacatcttaatc | |
| | | atgcctaggaagttttcta | |
| | | (SEQ ID NO: 24) | |
| L31-esaI | ttgacatcaggaaaatttttctgtataatgtgtgga | gggcccaagttcacttaaaaa | P186 |
| (P4/BCD22) | (SEQ ID NO: 19) | ggagatcaacaatgaaagcaa | primary |
| | | ttttcgtactgaaacatcttaatc | |
| | | atgcctaggaagttttcta | |
| | | (SEQ ID NO: 24) | |

Autonomous Knockdown of Pfk to Increase Myo-Inositol Production in E. coli

Strains containing autonomous Pfk knockdown using the Esa quorum sensing (QS) system were constructed. Strain IB2275 was used as a base strain into which EsaI expression cassettes for constitutive expression were integrated. A subset of promoters and RBS variants described by Mutalik et al. were used to provide a spectrum of expression levels for EsaI. Detailed genotype and explanation of the strains utilized are summarized in Table 5. In addition, a strain containing Pfk under the native promoter as the control strain (IB1379) was used. Production trials were carried out in modified MOPS minimal media, as herein.

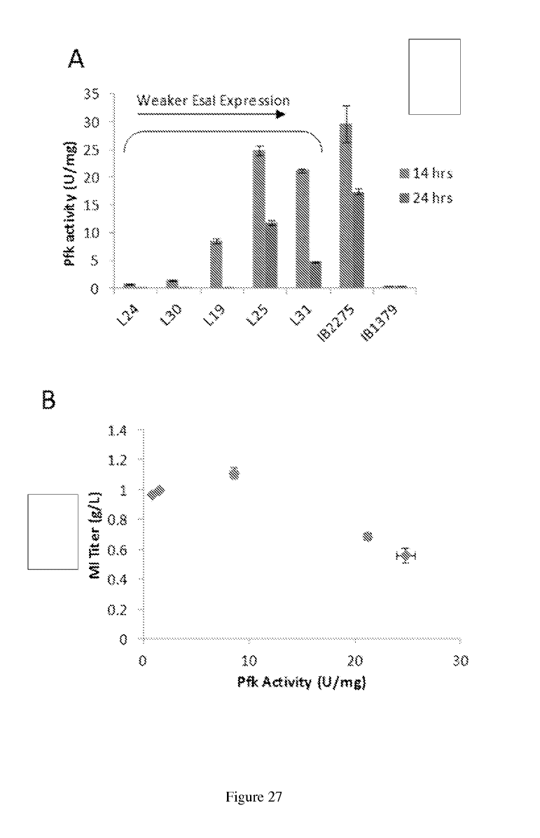

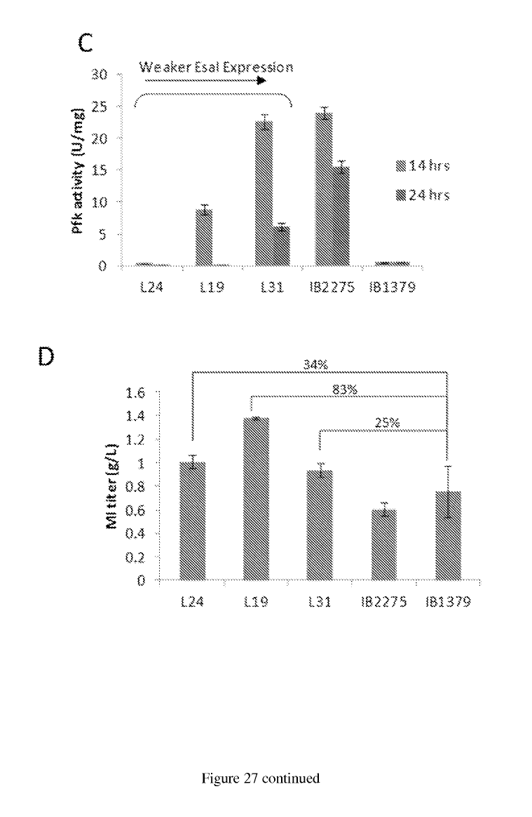

Strains containing various combinations of promoters and RBS variants to drive EsaI expression are designated as “LX”. EsaI expression varied across these strains, and thereby led to varied AHL production rates and varied rates of deactivation of the P_{esaS} promoter. FIG. 27A illustrates the measured Pfk activities in crude cell lysates at 14 hrs and 24 hrs into the fermentation. As EsaI expression decreases, the Pfk activity at a given time point is higher. The basal constitutive activity from the P_{esaS} promoter is higher than the activity from the native Pfk promoter, as can be seen by the differences between IB2275 and IB1379. By 24 hrs, the Pfk activity in L24, L30 and L19 dropped to below the constitutive levels of IB1379 and caused cell growth to cease.

Thus, expression of EsaI lead to AHL accumulation and strong deactivation of the P_{esaS} promoter. Thus, a series of strains were constructed with varying constitutive expression levels of EsaI that can switch from “growth mode” (high PfkA activity) to “production mode” (low PfkA activity) completely autonomously without any outside intervention or inducer addition. Such a series of strains can be utilized for production of myo-inositol, glucaric acid, or other products that may branch off from G6P or F6P. They are adaptable to a wide variety of applications as one can choose the optimal strain with the desired switching time when PfkA activity would be sufficiently suppressed. The production of myo-inositol (MI) was also tested from these strains to determine if the dynamic strategy gives an increase in titers. FIG. 27B shows that MI titers correlated with the Pfk activity at 14 hrs. Strain L19 led to a 20% increase in titers over strain IB1379. If Pfk activity was too high or too low, MI titers were not as high, presumably because the knockdown in Pfk activity happened too early or too late. Thus, this series of strains can be used to screen for the optimal rate for Pfk knockdown for a given pathway context of interest and to choose the optimal rate of downregulating glycolysis through Pfk to increase titers maximally.

In order to test the robustness of this strategy in different operational contexts, a subset of the above described strains were also tested in different media compositions. As many genetic pathways are strongly affected by operational conditions and nutrient conditions, it was demonstrated that this strategy is functional across a range of conditions. In addition to the trial in modified MOPS minimal media, production studies in MOPS media supplemented with 0.2% casamino acids were performed. This led to a slight increase in media composition. This could reduce lag phase in strains where Pfk activity was very low when transferred from an overnight culture, by allowing a few non-glucose dependent doublings while Pfk activity recovers. FIG. 27C shows Pfk activities at 14 hrs and 24 hours in MOPS+casamino acids media. Similar to the previous trial in MOPS media, Pfk activity at a given time trended with the EsaI expression level. By 24 hrs, activity in L24 and L19 had dropped below that in IB1379. This indicated that that QS-based autonomous knockdown of Pfk worked as well in this new richer media formulation. In addition, FIG. 27D shows that the increase in titers in this medium were significantly higher than in MOPS minimal medium. L19 showed an 83% increase in titers, while L24 and L31 showed a 34% and 25% increase in titers over IB1379.

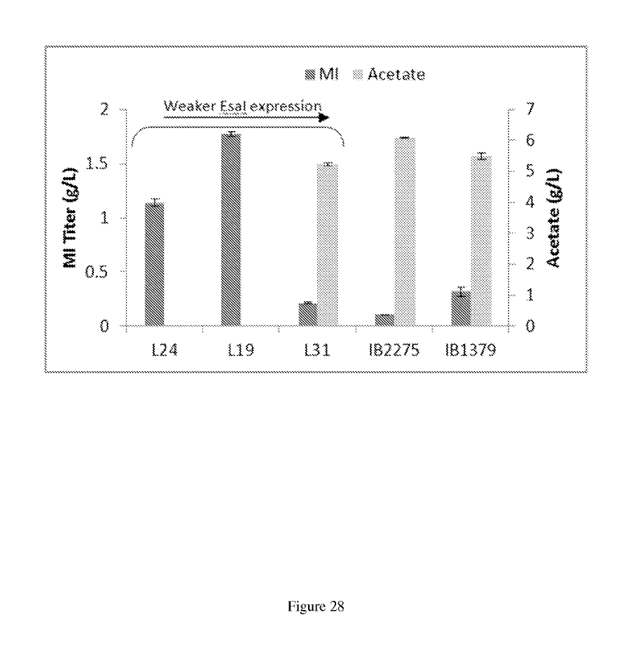

In addition to MOPS minimal media and MOPS minimal media+casamino acids, this system was also tested in a more industrially relevant media composition for production. T12 media (described in Irene Brockman's thesis) containing 10 g/L glucose was use. Some strains, especially those with a slow rate of Pfk knockdown or constitutive expression of Pfk, accumulated a significant amount of acetate in the fermentation. Strains containing a high enough expression of EsaI, such as L24 and L19, produced no detectable acetate and a much higher MI titer. This indicated that these strains were able to shut off Pfk expression fast enough to prevent acetate production from overflow metabolism and were able to direct G6P flux to MI production instead (FIG. 28). The remaining three strains, including the wildtype IB1379, had a Pfk activity that was high enough to cause excess flux through glycolysis and acetate production. In these cultures with excess acetate, the pH and final biomass were much lower, indicating that the acidic environment led to growth arrest and cell stress (FIG. 28). Thus, the autonomous system was able to not only allow downregulation of Pfk at various times, but also prevent unwanted acetate production if switched before the appropriate time.

TABLE 5
Description of Strains

| Strain | | |
|---|---|---|
| (designation) | Genotype | Description |
| IB1379 (WT) | MG1655 ΔendA Δzwf ΔpfkB | Wildtype control |
| IB2275 (No | MG1655 ΔendA Δzwf ΔpfkB pfkA::P_{EsaS}- | No EsaI control. Contains Pfk under |
| EsaI) | pfkA(LAA) HK022::apFAB104-EsaRI170V | the PesaS promoter, and EsaRI70V |
| | | under a constitutive bioFAB |
| | | promoter (apFAB104). Pfk is |
| | | constitutively expressed from the |
| | | activated PesaS promoter |
| IB2275 + L19 | MG1655 ΔendA Δzwf ΔpfkB pfkA::P_{EsaS}- | Predicted strength: 11. Strain |
| (L19) | pfkA(LAA) HK022::104-EsaRI170V | containing the IB2275 genetic |
| | 186(O)::apFAB296-abFAB700-EsaI | background with EsaI integrated |
| | | under one combination of promoters |
| | | and RBA variants proposed by |
| | | Mutalik et al. Production of AHL |
| | | from these strains leads to |
| | | attenuation of Pfk transcription and |
| | | reduction in its activity in the cell. |
| IB2275 + L24 | MG1655 ΔendA Δzwf ΔpfkB pfkA::P_{EsaS}- | Predicted strength: 30. See |
| (L24) | pfkA(LAA) HK022::104-EsaRI170V | description for L19 |
| | 186(O)::apFAB295-abFAB699-EsaI | |
| IB2275 + L25 | MG1655 ΔendA Δzwf ΔpfkB pfkA::P_{EsaS}- | Predicted strength: 8. See |
| (L25) | pfkA(LAA) HK022::104-EsaRI170V | description for L19 |
| | 186(O)::apFAB295-abFAB700-EsaI | |
| IB2275 + L30 | MG1655 ΔendA Δzwf ΔpfkB pfkA::P_{EsaS}- | Predicted strength: 18. See |
| (L30) | pfkA(LAA) HK022::104-EsaRI170V | description for L19 |
| | 186(O)::apFAB65-abFAB699-EsaI | |
| IB2275 + L31 | MG1655 ΔendA Δzwf ΔpfkB pfkA::P_{EsaS}- | Predicted strength: 7. See |
| (L31) | pfkA(LAA) HK022::104-EsaRI170V | description for L19 |
| | 186(O)::apFAB65-abFAB700-EsaI | |

### REFERENCES FOR EXAMPLE 3

Alper, H., Miyaoku, K., Stephanopoulos, G., 2005. Construction of lycopene-overproducing E. coli strains by combining systematic and combinatorial gene knockout targets. Nat Biotechnol. 23, 612-6.
Andersen, J. B., Sternberg, C., Poulsen, L. K., Bjorn, S. P., Givskov, M., Molin, S., 1998. New unstable variants of green fluorescent protein for studies of transient gene expression in bacteria. Appl Environ Microbiol. 64, 2240-6.
Anderson, J. C., Voigt, C. A., Arkin, A. P., 2007. Environmental signal integration by a modular AND gate. Molecular Systems Biology. 3, n/a-n/a.
Anesiadis, N., Cluett, W. R., Mahadevan, R., 2008. Dynamic metabolic engineering for increasing bioprocess productivity. Metab Eng. 10, 255-66.
Anesiadis, N., Kobayashi, H., Cluett, W. R., Mahadevan, R., 2013. Analysis and design of a genetic circuit for dynamic metabolic engineering. ACS Synth Biol. 2, 442-52.
Baba, T., Ara, T., Hasegawa, M., Takai, Y., Okumura, Y., Baba, M., Datsenko, K. A.,
Tomita, M., Wanner, B. L., Mori, H., 2006. Construction of Escherichia coli K-12 in-frame, single-gene knockout mutants: the Keio collection. Mol Syst Biol. 2, 2006 0008.
Balagadde, F. K., Song, H., Ozaki, J., Collins, C. H., Barnet, M., Arnold, F. H., Quake, S. R., You, L., 2008. A synthetic Escherichia coli predator-prey ecosystem. Mol Syst Biol. 4.
Balleza, E., Lopez-Bojorquez, L. N., Martinez-Antonio, A., Resendis-Antonio, O., Lozada-Chávez, I., Balderas-Martinez, Y. I., Encarnación, S., Collado-Vides, J., 2009. Regulation by transcription factors in bacteria: beyond description. FEMS Microbiology Reviews. 33, 133-151.
Bar-Even, A., Noor, E., Savir, Y., Liebermeister, W., Davidi, D., Tawfik, D. S., Milo, R., 2011. The Moderately Efficient Enzyme: Evolutionary and Physicochemical Trends Shaping Enzyme Parameters. Biochemistry. 50, 4402-4410.
Bayer, T. S., Smolke, C. D., 2005. Programmable ligand-controlled riboregulators of eukaryotic gene expression. Nat Biotech. 23, 337-343.
Bennett, B. D., Kimball, E. H., Gao, M., Osterhout, R., Van Dien, S. J., Rabinowitz, J. D., 2009. Absolute metabolite concentrations and implied enzyme active site occupancy in Escherichia coli. Nat Chem Biol. 5, 593-9.
Berger, S. A., Evans, P. R., 1991. Steady-state fluorescence of Escherichia coli phosphofructokinase reveals a regulatory role for ATP. Biochemistry. 30, 8477-80.
Bergmeyer, H. U., Bergmeyer, J., Grassl, M., 1983. Methods of enzymatic analysis/editor-in-chief, Hans Ulrich Bergmeyer; editors, Jürgen Bergmeyer and Marianne Grassl. Weinheim; Deerfield Beach, Fla.: Verlag Chemie, c1983-3rd ed.
Biggs, B. W., De Paepe, B., Santos, C. N. S., De Mey, M., Kumaran Ajikumar, P., 2014. Multivariate modular metabolic engineering for pathway and strain optimization. Current Opinion in Biotechnology. 29, 156-162.
BIOFAB, BIOFAB Data Access Client. 2012.
Blangy, D., Buc, H., Monod, J., 1968. Kinetics of the allosteric interactions of phosphofructokinase from Escherichia coli. J Mol Biol. 31, 13-35.
Bremer, H., Dennis, P. P., 1996. Escherichia coli and Salmonella typhimurium. In: Neidhardt F. C., C. R. I., Ingraham J. L., Lin E. C. C., Low K. B., et al., (Ed.), Cellular and Molecular Biology. American Society for Microbiology, Washington, D.C., pp. 1553-1569.
Brockman, I. M., Prather, K. L., 2015. Dynamic knockdown of E. coli central metabolism for redirecting fluxes of primary metabolites. Metab Eng. 28, 104-13.
Brockman, I. M. Dynamic control of Escherichia coli central metabolism for improvement of glucaric acid production by. (2015).
Callura, J. M., Cantor, C. R., Collins, J. J., 2012. Genetic switchboard for synthetic biology applications. Proceedings of the National Academy of Sciences of the United States of America. 109, 5850-5.
Cameron, D. E., Collins, J. J., 2014. Tunable protein degradation in bacteria. Nat Biotech. 32, 1276-1281.
Canonaco, F., Hess, T. A., Heri, S., Wang, T., Szyperski, T., Sauer, U., 2001. Metabolic flux response to phosphoglucose isomerase knock-out in Escherichia coli and impact of overexpression of the soluble transhydrogenase UdhA. FEMS Microbiol Lett. 204, 247-52.
Cardinale, S., Arkin, A. P., 2012. Contextualizing context for synthetic biology—identifying causes of failure of synthetic biological systems. Biotechnology Journal. 7, 856-866.
Chassagnole, C., Noisommit-Rizzi, N., Schmid, J. W., Mauch, K., Reuss, M., 2002. Dynamic modeling of the central carbon metabolism of Escherichia coli. Biotechnol Bioeng. 79, 53-73.
Chen, Z., Zeng, A.-P., 2013. Protein design in systems metabolic engineering for industrial strain development. Biotechnology Journal. 8, 523-533.
Collins, C. H., Arnold, F. H., Leadbetter, J. R., 2005. Directed evolution of Vibrio fischeri LuxR for increased sensitivity to a broad spectrum of acyl-homoserine lactones. Molecular Microbiology. 55, 712-723.
Dahl, R. H., Zhang, F., Alonso-Gutierrez, J., Baidoo, E., Batth, T. S., Redding-Johanson, A. M., Petzold, C. J., Mukhopadhyay, A., Lee, T. S., Adams, P. D., Keasling, J. D., 2013. Engineering dynamic pathway regulation using stress-response promoters. Nat Biotech. 31, 1039-1046.
Datsenko, K. A., Wanner, B. L., 2000. One-step inactivation of chromosomal genes in Escherichia coli K-12 using PCR products. Proceedings of the National Academy of Sciences of the United States of America. 97, 6640-5.
Davis, J. H., Baker, T. A., Sauer, R. T., 2011. Small-molecule control of protein degradation using split adaptors. ACS Chem Biol. 6, 1205-13.
De Anda, R., Lara, A. R., Hernández, V., Hernandez-Montalvo, V., Gosset, G., Bolívar, F., Ramírez, O. T., 2006. Replacement of the glucose phosphotransferase transport system by galactose permease reduces acetate accumulation and improves process performance of Escherichia coli for recombinant protein production without impairment of growth rate. Metabolic Engineering. 8, 281-290.
Delgado, J., Meruane, J., Liao, J. C., 1993. Experimental determination of flux control distribution in biochemical systems: in vitro model to analyze transient metabolite concentrations. Biotechnol Bioeng. 41, 1121-8.
Edwards, W. R., Williams, A. J., Morris, J. L., Baldwin, A. J., Allemann, R. K., Jones, D. D., 2010. Regulation of beta-lactamase activity by remote binding of heme: functional coupling of unrelated proteins through domain insertion. Biochemistry. 49, 6541-9.
Ellington, A. D., Szostak, J. W., 1990. In vitro selection of RNA molecules that bind specific ligands. Nature. 346, 818-822.
Farmer, W. R., Liao, J. C., 2000. Improving lycopene production in Escherichia coli by engineering metabolic control. Nat Biotechnol. 18, 533-7.
Fischer, E., Sauer, U., 2003. Metabolic flux profiling of Escherichia coli mutants in central carbon metabolism using GC-MS. European Journal of Biochemistry. 270, 880-891.
Franchini, A. G., Egli, T., 2006. Global gene expression in Escherichia coli K-12 during short-term and long-term adaptation to glucose-limited continuous culture conditions. Microbiology. 152, 2111-2127.
Gadkar, K. G., Doyle Iii, F. J., Edwards, J. S., Mahadevan, R., 2005. Estimating optimal profiles of genetic alterations using constraint-based models. Biotechnology and Bioengineering. 89, 243-251.
Gardner, T. S., Cantor, C. R., Collins, J. J., 2000. Construction of a genetic toggle switch in Escherichia coli. Nature. 403, 339-342.
Gonzalez, B., Francois, J., Renaud, M., 1997. A rapid and reliable method for metabolite extraction in yeast using boiling buffered ethanol. Yeast. 13, 1347-1355.
Guzman, L. M., Belin, D., Carson, M. J., Beckwith, J., 1995. Tight regulation, modulation, and high-level expression by vectors containing the arabinose PBAD promoter. Journal of Bacteriology. 177, 4121-30.
Hansen, C. A., Dean, A. B., Draths, K. M., Frost, J. W., 1999. Synthesis of 1,2,3,4-Tetrahydroxybenzene from d-Glucose:â [symbol] % Exploiting myo-Inositol as a Precursor to Aromatic Chemicals. Journal of the American Chemical Society. 121, 3799-3800.
Hanson, S., Berthelot, K., Fink, B., McCarthy, J. E. G., Suess, B., 2003. Tetracycline-aptamer-mediated translational regulation in yeast. Molecular Microbiology. 49, 1627-1637.
Haverkorn Van Rijsewijk, B. R. B., Nanchen, A., Nallet, S., Kleijn, R. J., Sauer, U., 2011. Large-scale 13C-flux analysis reveals distinct transcriptional control of respiratory and fermentative metabolism in Escherichia coli. Molecular Systems Biology. 7.
Hiller, J., Franco-Lara, E., Weuster-Botz, D., 2007. Metabolic profiling of Escherichia coli cultivations: evaluation of extraction and metabolite analysis procedures. Biotechnol Lett. 29, 1169-1178.
Holms, H., 1996. Flux analysis and control of the central metabolic pathways in Escherichia coli. FEMS Microbiology Reviews. 19, 85-116.
Holtz, W. J., Keasling, J. D., 2010. Engineering Static and Dynamic Control of Synthetic Pathways. Cell. 140, 19-23. http://partsregistry.org, Registry of Standard Biological Parts. Vol. 2011.
Hua, Q., Yang, C., Baba, T., Mori, H., Shimizu, K., 2003. Responses of the central metabolism in Escherichia coli to phosphoglucose isomerase and glucose-6-phosphate dehydrogenase knockouts. J Bacteriol. 185, 7053-67.
Ishii, N., Nakahigashi, K., Baba, T., Robert, M., Soga, T., Kanai, A., Hirasawa, T., Naba, M., Hirai, K., Hoque, A., Ho, P. Y., Kakazu, Y., Sugawara, K., Igarashi, S., Harada, S., Masuda, T., Sugiyama, N., Togashi, T., Hasegawa, M., Takai, Y., Yugi, K., Arakawa, K., Iwata, N., Toya, Y., Nakayama, Y., Nishioka, T., Shimizu, K., Mori, H., Tomita, M., 2007. Multiple High-Throughput Analyses Monitor the Response of E. coli to Perturbations. Science. 316, 593-597.
Johansson, L., Lindskog, A., Silfversparre, G., Cimander, C., Nielsen, K. F., Lidén, G., 2005. Shikimic acid production by a modified strain of E. coli (W3110.shik1) under phosphate-limited and carbon-limited conditions. Biotechnology and Bioengineering. 92, 541-552.
Jungbluth, M., Renicke, C., Taxis, C., 2010. Targeted protein depletion in Saccharomyces cerevisiae by activation of a bidirectional degron. BMC Syst Biol. 4, 176.
Kamionka, A., Bogdanska-Urbaniak, J., Scholz, O., Hillen, W., 2004. Two mutations in the tetracycline repressor change the inducer anhydrotetracycline to a corepressor. Nucleic Acids Research. 32, 842-847.
Karig, D., Weiss, R., 2005. Signal-amplifying genetic circuit enables in vivo observation of weak promoter activation in the Rhl quorum sensing system. Biotechnology and Bioengineering. 89, 709-718.
Keseler, I. M., Collado-Vides, J., Santos-Zavaleta, A., Peralta-Gil, M., Gama-Castro, S., Muñiz-Rascado, L., Bonavides-Martinez, C., Paley, S., Krummenacker, M., Altman, T., Kaipa, P., Spaulding, A., Pacheco, J., Latendresse, M., Fulcher, C., Sarker, M., Shearer, A. G., Mackie, A., Paulsen, I., Gunsalus, R. P., Karp, P. D., 2011. EcoCyc: a comprehensive database of Escherichia coli biology. Nucleic Acids Research. 39, D583-D590.
Kiely, D. E., Chen, L., Glucaric acid monoamides and their use to prepare poly(glucaramides). Google Patents, 1994.
Kim, J. Y. H., Cha, H. J., 2003. Down-regulation of acetate pathway through antisense strategy in Escherichia coli: Improved foreign protein production. Biotechnology and Bioengineering. 83, 841-853.
Klipp, E., Heinrich, R., Holzhütter, H.-G., 2002. Prediction of temporal gene expression. European Journal of Biochemistry. 269, 5406-5413.
Kobayashi, H., Kaern, M., Araki, M., Chung, K., Gardner, T. S., Cantor, C. R., Collins, J. J., 2004. Programmable cells: Interfacing natural and engineered gene networks. Proceedings of the National Academy of Sciences of the United States of America. 101, 8414-8419.
Kotlarz, D., Buc, H., 1982. [11] Phosphofructokinases from Escherichia coli. In: Willis, A. W., (Ed.), Methods in Enzymology. vol. Volume 90. Academic Press, pp. 60-70.
Kotlarz, D., Garreau, H., Buc, H., 1975. Regulation of the amount and of the activity of phosphofructokinases and pyruvate kinases in Escherichia coli. Biochimica et Biophysica Acta (BBA)—General Subjects. 381, 257-268.
Kuhlman, T. E., Cox, E. C., 2010. Site-specific chromosomal integration of large synthetic constructs. Nucleic Acids Res. 38, e92.
Laird, M. W., Sampey, G. C., Johnson, K., Zukauskas, D., Pierre, J., Hong, J. S., Cooksey,
B. A., Li, Y., Galperina, O., Karwoski, J. D., Burke, R. N., 2005. Optimization of BLyS production and purification from Escherichia coli. Protein Expression and Purification. 39, 237-246.
Lee, J., Natarajan, M., Nashine, V. C., Socolich, M., Vo, T., Russ, W. P., Benkovic, S. J., Ranganathan, R., 2008. Surface sites for engineering allosteric control in proteins. Science. 322, 438-42.
Lee, J. W., Kim, H. U., Choi, S., Yi, J., Lee, S. Y., 2011. Microbial production of building block chemicals and polymers. Curr Opin Biotechnol.
Lee, J. W., Na, D., Park, J. M., Lee, J., Choi, S., Lee, S. Y., 2012. Systems metabolic engineering of microorganisms for natural and non-natural chemicals. Nat Chem Biol. 8, 536-546.
Liu, H., Lu, T., 2015. Autonomous production of 1,4-butanediol via a de novo biosynthesis pathway in engineered Escherichia coli. Metab Eng.
Lucks, J. B., Qi, L., Mutalik, V. K., Wang, D., Arkin, A. P., 2011. Versatile RNA-sensing transcriptional regulators for engineering genetic networks. Proceedings of the National Academy of Sciences. 108, 8617-8622.
Lutz, R., Bujard, H., 1997. Independent and tight regulation of transcriptional units in Escherichia coli via the LacR/O, the TetR/O and AraC/I1-I2 regulatory elements. Nucleic Acids Res. 25, 1203-10.
Lynch, S. A., Gallivan, J. P., 2009. A flow cytometry-based screen for synthetic riboswitches. Nucleic Acids Research. 37, 184-192.
Majumder, A. L., Johnson, M. D., Henry, S. A., 1997. 1-myo-Inositol-1-phosphate synthase. Biochimica et Biophysica Acta (BBA)—Lipids and Lipid Metabolism. 1348, 245-256.
McGinness, K. E., Baker, T. A., Sauer, R. T., 2006. Engineering controllable protein degradation. Mol Cell. 22, 701-7.
Mehltretter, C. L., Rist, C. E., 1953. Sugar Oxidation, Saccharic and Oxalic Acids by the Nitric Acid Oxidation of Dextrose. Journal of Agricultural and Food Chemistry. 1, 779-783.
Michener, J. K., Thodey, K., Liang, J. C., Smolke, C. D., 2012. Applications of genetically-encoded biosensors for the construction and control of biosynthetic pathways. Metabolic Engineering. 14, 212-222.
Miksch, G., Bettenworth, F., Friehs, K., Flaschel, E., Saalbach, A., Twellmann, T., Nattkemper, T. W., 2005. Libraries of synthetic stationary-phase and stress promoters as a tool for fine-tuning of expression of recombinant proteins in Escherichia coli. Journal of Biotechnology. 120, 25-37.
Milias-Argeitis, A., Summers, S., Stewart-Ornstein, J., Zuleta, I., Pincus, D., El-Samad, H., Khammash, M., Lygeros, J., 2011. In silico feedback for in vivo regulation of a gene expression circuit. Nat Biotech. 29, 1114-1116.
Minogue, T. D., Trebra, M. W.-v., Bernhard, F., Bodman, S. B. v., 2002. The autoregulatory role of EsaR, a quorum-sensing regulator in Pantoea stewartii ssp. stewartii: evidence for a repressor function. Molecular Microbiology. 44, 1625-1635. Moon, T. S., Dueber, J. E., Shiue, E., Prather, K. L., 2010. Use of modular, synthetic scaffolds for improved production of glucaric acid in engineered E. coli. Metab Eng. 12, 298-305.
Moon, T. S., Yoon, S. H., Lanza, A. M., Roy-Mayhew, J. D., Prather, K. L., 2009. Production of glucaric acid from a synthetic pathway in recombinant Escherichia coli. Appl Environ Microbiol. 75, 589-95.
Morales, V. M., Backman, A., Bagdasarian, M., 1991. A series of wide-host-range low-copy-number vectors that allow direct screening for recombinants. Gene. 97, 39-47.
Morita, T., El-Kazzaz, W., Tanaka, Y., Inada, T., Aiba, H., 2003. Accumulation of glucose 6-phosphate or fructose 6-phosphate is responsible for destabilization of glucose transporter mRNA in Escherichia coli. The Journal of biological chemistry. 278, 15608-14.
Moser, F., Broers, N. J., Hartmans, S., Tamsir, A., Kerkman, R., Roubos, J. A., Bovenberg, R., Voigt, C. A., 2012. Genetic Circuit Performance under Conditions Relevant for Industrial Bioreactors. ACS Synthetic Biology. 1, 555-564.
Mutalik, V. K., Guimaraes, J. C., Cambray, G., Lam, C., Christoffersen, M. J., Mai, Q.-A., Tran, A. B., Paull, M., Keasling, J. D., Arkin, A. P., Endy, D., 2013. Precise and reliable gene expression via standard transcription and translation initiation elements. Nat Meth. 10, 354-360.
Nakajima, M., Imai, K., Ito, H., Nishiwaki, T., Murayama, Y., Iwasaki, H., Oyama, T., Kondo, T., 2005. Reconstitution of Circadian Oscillation of Cyanobacterial KaiC Phosphorylation in Vitro. Science. 308, 414-415.
Nakamura, C. E., Whited, G. M., 2003. Metabolic engineering for the microbial production of 1,3-propanediol. Current Opinion in Biotechnology. 14, 454-459.
Neubauer, P., Junne, S., 2010. Scale-down simulators for metabolic analysis of large-scale bioprocesses. Current Opinion in Biotechnology. 21, 114-121.
Ogawa, T., Mori, H., Tomita, M., Yoshino, M., 2007 Inhibitory effect of phosphoenolpyruvate on glycolytic enzymes in Escherichia coli. Res Microbiol. 158, 159-63.
Ostermeier, M., 2005. Engineering allosteric protein switches by domain insertion. Protein engineering, design & selection: PEDS. 18, 359-64.
Patrick, W. M., Quandt, E. M., Swartzlander, D. B., Matsumura, I., 2007. Multicopy Suppression Underpins Metabolic Evolvability. Molecular Biology and Evolution. 24, 2716-2722.
Peti, W., Page, R., 2007. Strategies to maximize heterologous protein expression in Escherichia coli with minimal cost. Protein Expression and Purification. 51, 1-10.
Pósfai, G., Plunkett, G., Fehér, T., Frisch, D., Keil, G. M., Umenhoffer, K., Kolisnychenko, V., Stahl, B., Sharma, S. S., de Arruda, M., Burland, V., Harcum, S. W., Blattner, F. R., 2006. Emergent Properties of Reduced-Genome Escherichia coli. Science. 312, 1044-1046.
Quan, J., Tian, J., 2009. Circular Polymerase Extension Cloning of Complex Gene Libraries and Pathways. PLoS ONE. 4, e6441.
Raman, B., Nandakumar, M. P., Muthuvijayan, V., Marten, M. R., 2005. Proteome analysis to assess physiological changes in Escherichia coli grown under glucose-limited fed-batch conditions. Biotechnology and Bioengineering. 92, 384-392.
Raman, S., Rogers, J. K., Taylor, N. D., Church, G. M., 2014. Evolution-guided optimization of biosynthetic pathways. Proceedings of the National Academy of Sciences. 111, 17803-17808.
Reisch, C. R., Prather, K. L. J., in preparation. Cas9 Endonuclease as Counter-Selectable Marker for Scarless Genome Editing in E. coli.
Ro, D.-K., Paradise, E. M., Ouellet, M., Fisher, K. J., Newman, K. L., Ndungu, J. M., Ho, K. A., Eachus, R. A., Ham, T. S., Kirby, J., Chang, M. C. Y., Withers, S. T., Shiba, Y., Sarpong, R., Keasling, J. D., 2006. Production of the antimalarial drug precursor artemisinic acid in engineered yeast. Nature. 440, 940-943.
Ryu, M.-H., Gomelsky, M., 2014. Near-infrared Light Responsive Synthetic c-di-GMP Module for Optogenetic Applications. ACS Synthetic Biology. 3, 802-810.
Saeidi, N., Wong, C. K., Lo, T.-M., Nguyen, H. X., Ling, H., Leong, S. S. J., Poh, C. L.,
Chang, M. W., 2011. Engineering microbes to sense and eradicate Pseudomonas aeruginosa, a human pathogen. Mol Syst Biol. 7.
Salis, H. M., 2011. Chapter two—The Ribosome Binding Site Calculator. In: Christopher, V., (Ed.), Methods in Enzymology. vol. Volume 498. Academic Press, pp. 19-42.
Santos, C. N. S., Stephanopoulos, G., 2008. Combinatorial engineering of microbes for optimizing cellular phenotype. Current Opinion in Chemical Biology. 12, 168-176.
Sanwal, B. D., 1970. Regulatory Mechanisms Involving Nicotinamide Adenine Nucleotides as Allosteric Effectors 0.3. Control of Glucose 6-Phosphate Dehydrogenase. Journal of Biological Chemistry. 245, 1626-&.
Scalcinati, G., Knuf, C., Partow, S., Chen, Y., Maury, J., Schalk, M., Daviet, L., Nielsen, J., Siewers, V., 2012. Dynamic control of gene expression in Saccharomyces cerevisiae engineered for the production of plant sesquitepene alpha-santalene in a fed-batch mode. Metab Eng. 14, 91-103.
Schallmey, M., Frunzke, J., Eggeling, L., Marienhagen, J., 2014. Looking for the pick of the bunch: high-throughput screening of producing microorganisms with biosensors. Current Opinion in Biotechnology. 26, 148-154.
Schu, D. J., Carlier, A. L., Jamison, K. P., von Bodman, S., Stevens, A. M., 2009. Structure/Function Analysis of the Pantoea stewartii Quorum-Sensing Regulator EsaR as an Activator of Transcription. Journal of Bacteriology. 191, 7402-7409.
Serganov, A., Patel, D. J., 2012. Metabolite Recognition Principles and Molecular Mechanisms Underlying Riboswitch Function. Annual Review of Biophysics. 41, 343-370.
Sharma, V., Nomura, Y., Yokobayashi, Y., 2008. Engineering Complex Riboswitch Regulation by Dual Genetic Selection. Journal of the American Chemical Society. 130, 16310-16315.
Shetty, R. P., Endy, D., Knight, T. F., Jr., 2008. Engineering BioBrick vectors from BioBrick parts. J Biol Eng. 2, 5.
Shin, P. K., Seo, J.-H., 1990. Analysis of E. coli phoA-lacZ fusion gene expression inserted into a multicopy plasmid and host cell's chromosome. Biotechnology and Bioengineering. 36, 1097-1104.
Shiue, E., Brockman, I. M., Prather, K. L., 2015. Improving product yields on D-glucose in Escherichia coli via knockout of pgi and zwf and feeding of supplemental carbon sources. Biotechnol Bioeng. 112, 579-87.
Shiue, E., Prather, K. L., 2014. Improving D-glucaric acid production from myo-inositol in E. coli by increasing MIOX stability and myo-inositol transport. Metab Eng. 22, 22-31.
Shiue, E., Prather, K. L. J., 2012. Synthetic biology devices as tools for metabolic engineering. Biochemical Engineering Journal. 65, 82-89.
Shong, J., Collins, C. H., 2013. Engineering the esaR Promoter for Tunable Quorum Sensing-Dependent Gene Expression. ACS Synthetic Biology. 2, 568-575.
Shong, J., Huang, Y.-M., Bystroff, C., Collins, C. H., 2013. Directed Evolution of the Quorum-Sensing Regulator EsaR for Increased Signal Sensitivity. ACS Chemical Biology. 8, 789-795.
Smanski, M. J., Bhatia, S., Zhao, D., Park, Y., B A Woodruff, L., Giannoukos, G., Ciulla, D., Busby, M., Calderon, J., Nicol, R., Gordon, D. B., Densmore, D., Voigt, C. A., 2014. Functional optimization of gene clusters by combinatorial design and assembly. Nat Biotech. 32, 1241-1249.
Smith, T. N., Kiely, D. E., Kramer-Presta, K., Corrosion inhibiting composition. Google Patents, 2012.
Solomon, K. V., Moon, T. S., Ma, B., Sanders, T. M., Prather, K. L. J., 2012a. Tuning Primary Metabolism for Heterologous Pathway Productivity. ACS Synthetic Biology.
Solomon, K. V., Sanders, T. M., Prather, K. L., 2012b. A dynamic metabolite valve for the control of central carbon metabolism. Metab Eng. 14, 661-71.
Soma, Y., Tsuruno, K., Wada, M., Yokota, A., Hanai, T., 2014. Metabolic flux redirection from a central metabolic pathway toward a synthetic pathway using a metabolic toggle switch. Metab Eng. 23, 175-84.
Soukup, G. A., Breaker, R. R., 1999. Engineering precision RNA molecular switches. Proceedings of the National Academy of Sciences. 96, 3584-3589.
Sowa, S. W., Baldea, M., Contreras, L. M., 2014. Optimizing Metabolite Production Using Periodic Oscillations. PLoS Comput Biol. 10, e1003658.
St-Pierre, F., Cui, L., Priest, D. G., Endy, D., Dodd, I. B., Shearwin, K. E., 2013. One-step cloning and chromosomal integration of DNA. ACS Synth Biol. 2, 537-41.
Stephanopoulos, G., Aristidou, A. A., Nielsen, J. H., 1998. Metabolic engineering: principles and methodologies. Academic Press, San Diego.
Stevens, J. T., Carothers, J. M., 2015. Designing RNA-Based Genetic Control Systems for Efficient Production from Engineered Metabolic Pathways. ACS Synth Biol. 4, 107-15.
Swartz, J. R., 2001. Advances in Escherichia coli production of therapeutic proteins. Current Opinion in Biotechnology. 12, 195-201.
Torella, J. P., Ford, T. J., Kim, S. N., Chen, A. M., Way, J. C., Silver, P. A., 2013. Tailored fatty acid synthesis via dynamic control of fatty acid elongation. Proceedings of the National Academy of Sciences. 110, 11290-11295.
Tsao, C.-Y., Hooshangi, S., Wu, H.-C., Valdes, J. J., Bentley, W. E., 2010. Autonomous induction of recombinant proteins by minimally rewiring native quorum sensing regulon of E. coli. Metabolic Engineering. 12, 291-297.
Tseng, H.-C., Martin, C. H., Nielsen, D. R., Prather, K. L. J., 2009. Metabolic Engineering of Escherichia coli for Enhanced Production of (R)- and (S)-3-Hydroxybutyrate. Applied and Environmental Microbiology. 75, 3137-3145.
Tyo, K. E. J., Kocharin, K., Nielsen, J., 2010. Toward design-based engineering of industrial microbes. Current Opinion in Microbiology. 13, 255-262.
Walaszek, Z., 1990. Potential use of d-glucaric acid derivatives in cancer prevention. Cancer Letters. 54, 1-8.
Wang, H. H., Kim, H., Cong, L., Jeong, J., Bang, D., Church, G. M., 2012. Genome-scale promoter engineering by coselection MAGE. Nat Meth. 9, 591-593.
Werpy, T., Petersen, G., Top value added chemical from biomass, volume 1-results of screening for potential candidates from sugars and synthetic gas. U.S. Department of Energy, Washington, D.C., 2004.
Wessely, F., Bartl, M., Guthke, R., Li, P., Schuster, S., Kaleta, C., 2011. Optimal regulatory strategies for metabolic pathways in Escherichia coli depending on protein costs. Mol Syst Biol. 7, 515.
Woolston, B. M., Edgar, S., Stephanopoulos, G., 2013. Metabolic Engineering: Past and Future. Annual Review of Chemical and Biomolecular Engineering. 4, 259-288.
Xie, W., Ye, L., Lv, X., Xu, H., Yu, H., 2015. Sequential control of biosynthetic pathways for balanced utilization of metabolic intermediates in Saccharomyces cerevisiae. Metabolic Engineering. 28, 8-18.
Xu, B., Jahic, M., Blomsten, G., Enfors, S. O., 1999. Glucose overflow metabolism and mixed-acid fermentation in aerobic large-scale fed-batch processes with Escherichia coli. Applied Microbiology and Biotechnology. 51, 564-571.
Xu, P., Li, L., Zhang, F., Stephanopoulos, G., Koffas, M., 2014. Improving fatty acids production by engineering dynamic pathway regulation and metabolic control. Proceedings of the National Academy of Sciences. 111, 11299-11304.
Xu, P., Wang, W., Li, L., Bhan, N., Zhang, F., Koffas, M. A. G., 2013. Design and Kinetic Analysis of a Hybrid Promoter-Regulator System for Malonyl-CoA Sensing in Escherichia coli. ACS Chemical Biology. 9, 451-458.
Yamaoka, M., Osawa, S., Morinaga, T., Takenaka, S., Yoshida, K.-i., 2011. A cell factory of Bacillus subtilis engineered for the simple bioconversion of myo-inositol to scyllo-inositol, a potential therapeutic agent for Alzheimer's disease. Microbial Cell Factories. 10, 1-6.
Ye, H., Baba, M. D.-E., Peng, R.-W., Fussenegger, M., 2011. A Synthetic Optogenetic Transcription Device Enhances Blood-Glucose Homeostasis in Mice. Science. 332, 1565-1568.
Yokobayashi, Y., Weiss, R., Arnold, F. H., 2002. Directed evolution of a genetic circuit. Proceedings of the National Academy of Sciences. 99, 16587-16591.
Yoo, S. M., Na, D., Lee, S. Y., 2013. Design and use of synthetic regulatory small RNAs to control gene expression in Escherichia coli. Nat. Protocols. 8, 1694-1707.
Yoon, S.-H., Moon, T. S., Iranpour, P., Lanza, A. M., Prather, K. J., 2009. Cloning and Characterization of Uronate Dehydrogenases from Two Pseudomonads and Agrobacterium tumefaciens Strain C58. Journal of Bacteriology. 191, 1565-1573.
Yoon, S. K., Kang, W. K., Park, T. H., 1996. Regulation of trp promoter for production of bovine somatotropin in recombinant Escherichia coli fed-batch fermentation. Journal of Fermentation and Bioengineering. 81, 153-157.
Youngquist, J. T., Rose, J., Pfleger, B., 2013. Free fatty acid production in Escherichia coli under phosphate-limited conditions. Applied Microbiology and Biotechnology. 97, 5149-5159.
Zaslaver, A., Bren, A., Ronen, M., Itzkovitz, S., Kikoin, I., Shavit, S., Liebermeister, W., Surette, M. G., Alon, U., 2006. A comprehensive library of fluorescent transcriptional reporters for Escherichia coli. Nat Meth. 3, 623-628.
Zaslaver, A., Mayo, A. E., Rosenberg, R., Bashkin, P., Sberro, H., Tsalyuk, M., Surette, M. G., Alon, U., 2004. Just-in-time transcription program in metabolic pathways. Nat Genet. 36, 486-491.
Zhang, F., Carothers, J. M., Keasling, J. D., 2012. Design of a dynamic sensor-regulator system for production of chemicals and fuels derived from fatty acids. Nat Biotech. 30, 354-359.
Zhang, F., Keasling, J., 2011. Biosensors and their applications in microbial metabolic engineering. Trends in Microbiology. 19, 323-329.
Zhao, J., Baba, T., Mori, H., Shimizu, K., 2004. Effect of zwf gene knockout on the metabolism of Escherichia coli grown on glucose or acetate. Metabolic Engineering. 6, 164-174.
Zhu, Y., Eiteman, M. A., Altman, R., Altman, E., 2008. High Glycolytic Flux Improves Pyruvate Production by a Metabolically Engineered Escherichia coli Strain. Applied and Environmental Microbiology. 74, 6649-6655.
Zor, T., Selinger, Z., 1996. Linearization of the Bradford Protein Assay Increases Its Sensitivity: Theoretical and Experimental Studies. Analytical Biochemistry. 236, 302-308.

### Example 4: In Vitro Degradation of Tagged Pfk-I

### Materials and Methods

An in vitro assay of Pfk-I degradation was carried out to confirm that the expected degradation of the protein occurred in the presence of ClpXP and ATP. This would alleviate concerns that the structure of Pfk-I limited accessibility of the degradation tag or promoted cleavage of the tag without unfolding and degradation of the remaining protein.

### Purification of Degradation Tagged Pfk-I

N-terminal 6x-His-tagged versions of Pfk-I (with and without C terminal degradation tags) were overexpressed in DH10B in LB from pMMB206-based plasmids. Protein was purified from each culture using QIAGEN Ni-NTA spin columns and following the standard protocol recommended in the kit handbook for protein purification under native conditions. Eluted protein samples were dialyzed overnight at 4° C. into a buffer containing 20 mM HEPES, 400 mM NaCl, 100 mM KCl, and 10% glycerol (pH 7.6). Protein concentrations were measured by Bradford assay.

### In Vitro Assay of Pfk-I Degradation

The in vitro degradation assay was carried out via a protocol developed by the Sauer Lab at MIT (Department of Biology). Each reaction contained 0.3 μM ClpX6 and 0.9 μM ClpP14 and 10 μM of the Pfk-I variant of interest in PD buffer (25 mM HEPES, 100 mM KCl, 10 mM MgCl2, 10% glycerol). The reaction mixture also contained an ATP regeneration system consisting of 5 mM ATP, 0.032 mg/ml creatine kinase, and 16 mM phosphocreatine. Reactions were sampled at 0, 2, 5, 10, 20, 30, 45, and 60 minutes and quenched by dilution into an equal volume of SDS-PAGE buffer (65.8 mM Tris-HCl, pH 6.8, 2.1% SDS, 26.3% (w/v) glycerol, 0.01% bromophenol blue, 5% f3-mercaptoethanol) followed by heating at 95° C. for 5 minutes. Samples were resolved by SDS-PAGE.

### Results

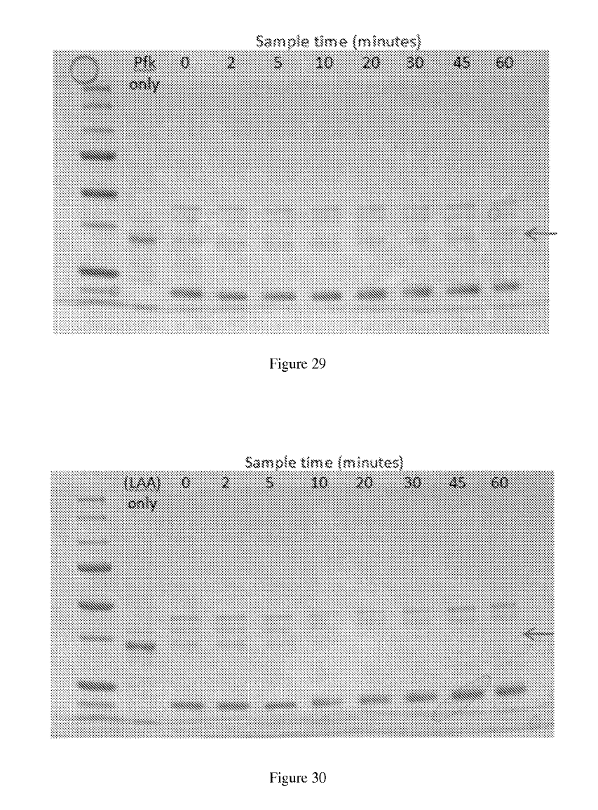

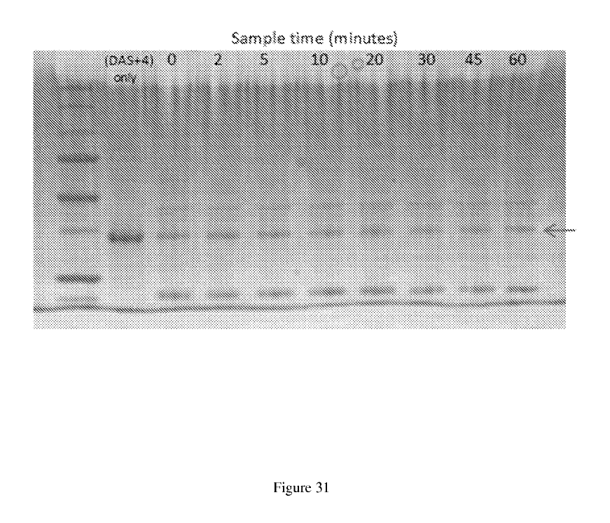

Untagged Pfk-I (FIG. 28) does not show significant degradation during the 60 minute incubation with ClpXP. In contrast, Pfk-I with the native ssrA tag (LAA) appended to the C terminus shows almost complete disappearance by 20 minutes (FIG. 29), indicating strong degradation of the tagged protein. With the modified DAS+4 tag, no significant degradation was observed over the 60 minute period (FIG. 30), which was the expected outcome in the absence of SspB. These results indicated that degradation strategies based on addition of a C-terminal ssrA tag to Pfk-I should be feasible.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

1. A method of redirecting flux of glucose-6-phosphate in a recombinant cell, the method comprising regulating activity of a phosphofructokinase-1 (pfk-1) in the recombinant cell.

2. The method of claim 1, further comprising expressing in the cell a heterologous pathway that can utilize a glycolytic intermediate.

3. The method of claim 1 or 2, wherein the glycolytic intermediate is glucose-6-phosphate.

4. The method of any one of claims 1-3, wherein the heterologous pathway comprises expressing a myo-inositol-1-phosphate synthase.

5. The method of any one of claims 1-4, further comprising reducing expression of a glucose-6-phosphate dehydrogenase (zwf).

6. The method of any one of claims 1-5, wherein the cell does not express glucose-6-phosphate dehydrogenase.

7. The method of any one of claims 1-6, wherein regulating activity of the phosphofructokinase-1 protein comprises reducing the amount of phosphofructokinase-1 protein in the cell.

8. The method of claim 7, wherein the amount of phosphofructokinase-1 protein is reduced by at least 50%.

9. The method of claim 7, wherein the amount of phosphofructokinase-1 protein is reduced by at least 75%.

10. The method of claim 7, wherein the amount of phosphofructokinase-1 protein is reduced by at least 90%.

11. The method of any one of claims 7-10, wherein reducing the amount of phosphofructokinase-1 protein comprises degrading the phosphofructokinase-1 protein.

12. The method of any one of claims 7-11, wherein reducing the amount of phosphofructokinase-1 protein comprises targeting the phosphofructokinase-1 protein for degradation by a protease.

13. The method of any one of claims 1-12, wherein the phosphofructokinase-1 protein is fused to a peptide tag.

14. The method of claim 13, wherein the peptide tag is an SsrA tag.

15. The method of any one of claims 1-14, further comprising expressing in the cell an adaptor protein.

16. The method of claim 15, wherein the adaptor protein is SspB and targets the phosphofructokinase-1 protein for degradation.

17. The method of claim A16, wherein SspB is expressed under the control of a first inducible promoter.

18. The method of any one of claims 1-17, further comprising contacting the cell with a first inducer.

19. The method of any one of claims 4-18, wherein the myo-inositol-1-phosphate synthase (INO1) is expressed under the control of an inducible promoter.

20. The method of claim 19, further comprising contacting the cell with a second inducer.

21. The method of claim 18, wherein the first inducer is anhydrotetracycline (aTc).

22. The method of claim 20, wherein the second inducer is isopropyl-β-D-1-thiogalactopyranoside.

23. The method of any one of claims 4-22, wherein the gene encoding the myo-inositol-1-phosphate synthase is a Saccharomyces gene.

24. The method of any one of claims 1-23, wherein the cell is a microbial cell.

25. The method of claim 24, wherein the microbial cell is a bacterial cell.

26. The method of claim 25, wherein the bacterial cell is an Escherichia coli cell.

27. The method of any one of claims 1-23, wherein the cell is a eukaryotic cell.

28. The method of claim 27, wherein the eukaryotic cell is a fungal cell, a yeast cell, an insect cell, a plant cell, or a mammalian cell.

29. The method of any one of claims 1-28, wherein the method is a method of producing myo-inositol, wherein the method further comprises culturing the cell and optionally recovering myo-inositol from the cell and/or cell culture.

30. The method of any one of claims 1-29, further comprising expressing in the cell a gene encoding a myo-inositol oxygenase.

31. The method of any one of claims 1-30, further comprising expressing in the cell a gene encoding a uronate dehydrogenase.

32. The method of any one of claims 1-31, wherein the method is a method of producing glucuronic acid, and the method further comprises culturing the cell and optionally recovering glucuronic acid from the cell and/or cell culture.

33. The method of any one of claims 1-32, wherein the method is a method of producing glucaric acid, and the method further comprises culturing the cell and optionally recovering glucaric acid from the cell and/or cell culture.

34. The method of any one of claims 1-33, wherein the method further comprises reducing expression of a glucarate dehydratase protein.

35. The method of any one of claims 1-34, wherein the method further comprises mutating a gudD gene in the recombinant cell.

36. The method of any one of claims 1-35, wherein the method further comprises reducing the expression of a uronate isomerase protein.

37. The method of any one of claims 1-36, wherein the method further comprises mutating a uxaC gene in the recombinant cell.

38. The method of any one of claims 1-37, further comprising reducing expression of the phosphofructokinase-II protein.

39. The method of claim 38, wherein reducing expression of the phosphofructokinase-II protein comprises eliminating expression the phosphofructokinase-II protein.

40. The method of any one of claims 15-39, wherein a gene encoding the adaptor protein is integrated into the genome of the cell.

41. The method of claim 40, wherein the gene encoding the adaptor protein is integrated at a phage attachment site.

42. The method of claim 41, wherein the phage attachment site is HK022.

43. A method for producing a recombinant cell, comprising expressing in the cell a regulatable phosphofructokinase protein (Pfk-1) and a means of regulating the phosphofructokinase protein.

44. The method of claim 43, further comprising expressing in the cell a heterologous pathway that can utilize a glycolytic intermediate.

45. The method of claim 44, wherein the glycolytic intermediate is glucose-6-phosphate.

46. The method of any one of claims 43-45, further comprising expressing in the cell a myo-inositol-1-phosphate synthase (INO1).

47. The method of any one of claims 43-46, further comprising reducing expression of a glucose-6-phosphate dehydrogenase gene (zwf).

48. The method of any one of claims 43-47, wherein the method further comprises reducing expression of a glucarate dehydratase protein.

49. The method of any one of claims 43-48, wherein the method further comprises mutating a gudD gene in the recombinant cell.

50. The method of any one of claims 43-49, wherein the method further comprises reducing the expression of a uronate isomerase protein.

51. The method of any one of claims 43-50, wherein the method further comprises mutating a uxaC gene in the recombinant cell.

52. The method of any one of claims 43-51, wherein the cell is cultured in the presence of glucose.

53. The method of any one of claims 43-52, wherein the cell is cultured in the presence of arabinose.

54. The method of any one of claims 43-53, wherein the cell is cultured in the presence of xylose.

55. The method of any one of claims 43-54, wherein the cell is a microbial cell.

56. The method of claim 55, wherein the microbial cell is a bacterial cell.

57. The method of claim 56, wherein the bacterial cell is an Escherichia coli cell.

58. The method of any one of claims 43-54, wherein the cell is a eukaryotic cell.

59. The method of claim 58, wherein the eukaryotic cell is a fungal cell, a yeast cell, an insect cell, a plant cell, or a mammalian cell.

60. A recombinant cell that expresses a regulatable phosphofructokinase protein (Pfk-1) and a means of regulating the phosphofructokinase protein.

61. The recombinant cell of claim 60, wherein the cell further expresses a heterologous pathway that can utilize a glycolytic intermediate.

62. The recombinant cell of claim 61, wherein the glycolytic intermediate is glucose-6-phosphate.

63. The recombinant cell of any one of claims 60-62, wherein the cell further expresses a myo-inositol-1-phosphate synthase (INO1).

64. The recombinant cell of any one of claims 60-63, wherein the cell has reduced expression of a glucose-6-phosphate dehydrogenase gene (zwf).

65. The recombinant cell of any one of claims 60-64, wherein the cell does not express glucose-6-phosphate dehydrogenase.

66. The recombinant cell of any one of claims 60-65, wherein the amount of the phosphofructokinase-1 protein is reduced in the cell.

67. The recombinant cell of claim 66, wherein the amount of phosphofructokinase-1 protein is reduced by at least 50%.

68. The recombinant cell of claim 66, wherein the amount of phosphofructokinase-1 protein is reduced by at least 75%.

69. The recombinant cell of claim 66, wherein the amount of phosphofructokinase-1 protein is reduced by at least 90%.

70. The recombinant cell of any one of claims 66-69, wherein the amount of phosphofructokinase-1 protein is reduced by degrading the phosphofructokinase-1 protein.

71. The recombinant cell of any one of claims 66-70, wherein the amount of phosphofructokinase-1 protein is reduced by targeting the phosphofructokinase-1 protein for degradation by a protease.

72. The recombinant cell of any one of claims 60-71, wherein the phosphofructokinase-1 protein is fused to a peptide tag.

73. The recombinant cell of claim 72, wherein the peptide tag is an SsrA tag.

74. The recombinant cell of any one of claims 60-73, wherein the cell expresses an adaptor protein.

75. The recombinant cell of claim 74, wherein the adaptor protein is SspB and targets the phosphofructokinase-1 protein for degradation.

76. The recombinant cell of claim 75, wherein SspB is expressed under the control of a first inducible promoter.

77. The recombinant cell of claim 76, wherein the cell is contacted with a first inducer.

78. The recombinant cell of any one of claims 63-77, wherein the myo-inositol-1-phosphate synthase (INO1) is expressed under the control of an inducible promoter.

79. The recombinant cell of claim 78, wherein the cell is contacted with a second inducer.

80. The recombinant cell of claim 77, wherein the first inducer is anhydrotetracycline (aTc).

81. The recombinant cell of claim 79, wherein the second inducer is isopropyl-β-D-1-thiogalactopyranoside (IPTG).

82. The recombinant cell of any one of claims 63-81, wherein the gene encoding the myo-inositol-1-phosphate synthase is a Saccharomyces gene.

83. The recombinant cell of any one of claims 60-82, wherein the cell is a microbial cell.

84. The recombinant cell of claim 83, wherein the microbial cell is a bacterial cell.

85. The recombinant cell of claim 84, wherein the bacterial cell is an Escherichia coli cell.

86. The recombinant cell of any one of claims 60-82, wherein the cell is a eukaryotic cell.

87. The recombinant cell of claim 86, wherein the eukaryotic cell is a fungal cell, a yeast cell, an insect cell, a plant cell, or a mammalian cell.

88. The recombinant cell of any one of claims 60-87, wherein the cell expresses a gene encoding a myo-inositol oxygenase.

89. The recombinant cell of any one of claims 60-88, wherein the cell expresses a gene encoding a uronate dehydrogenase.

90. The recombinant cell of any one of claims 60-89, wherein the cell has reduced expression of the phosphofructokinase-II protein.

91. The recombinant cell of claim 90, wherein the cell does not express phosphofructokinase-II.

92. The recombinant cell of any one of claims 60-91, wherein the cell has reduced expression of glucarate dehydratase.

93. The recombinant cell of any one of claims 60-92, wherein the cell does not express glucarate dehydratase.

94. The recombinant cell of any one of claims 60-93, wherein an endogenous gudD gene of the cell is mutated.

95. The recombinant cell of any one of claims 60-94, wherein the cell has reduced expression of uronate isomerase.

96. The recombinant cell of any one of claims 60-95, wherein the cell does not express uronate isomerase.

97. The recombinant cell of any one of claims 60-96, wherein an endogenous uxaC gene of the cell is mutated.

98. The recombinant cell of any one of claims 72-97, wherein a gene encoding the adaptor protein is integrated into the genome of the cell.

99. The recombinant cell of claim 98, wherein the gene encoding the adaptor protein is integrated at a phage attachment site.

100. The recombinant cell of claim 99, wherein the phage attachment site is HK022.

101. A method of producing myo-inositol, the method comprising culturing the cell of any one of claim 60-100, or 191 to produce myo-inositol.

102. The method of claim 101, further comprising recovering the myo-inositol from the cell culture.

103. The method of claim 101 or 102, wherein the cell is cultured in the presence of glucose.

104. The method of any one of claims 101-103, wherein the cell is cultured in the presence of arabinose.

105. The method of any one of claims 101-104, wherein the cell is cultured in the presence of xylose.

106. A method of producing glucuronic acid or glucaric acid, the method comprising culturing the cell of any one of claim 60-100, or 191 to produce glucuronic acid or glucaric acid.

107. The method of claim 106, further comprising recovering the glucuronic acid or glucaric acid from the cell culture.

108. The method of claim 106 or 107, wherein the cell is cultured in the presence of glucose.

109. The method of any one of claims 106-108, wherein the cell is cultured in the presence of arabinose.

110. The method of any one of claims 106-109, wherein the cell is cultured in the presence of xylose.

111. A cell culture produced by culturing the cell of any one of claims 60-100 or 191.

112. The cell culture of claim 111, wherein the cell culture contains at least 100 mg L⁻¹ myo-inositol.

113. The cell culture of claim 111, wherein the cell culture contains at least 500 mg L⁻¹ myo-inositol.

114. The cell culture of claim 11, wherein the cell culture contains at least 100 mg L⁻¹ glucuronic acid.

115. The cell culture of claim 11, wherein the cell culture contains at least 500 mg L⁻¹ glucuronic acid.

116. The cell culture of claim 11, wherein the cell culture contains at least 100 mg L⁻¹ glucaric acid.

117. The cell culture of claim 11, wherein the cell culture contains at least 500 mg L⁻¹ glucaric acid.

118. A supernatant of a cell culture produced by culturing the cell of any one of claim 60-100 or 191.

119. The supernatant of claim 118, wherein the supernatant contains at least 100 mg L⁻¹ myo-inositol.

120. The supernatant of claim 118, wherein the supernatant contains at least 500 mg L⁻¹ myo-inositol.

121. The supernatant of claim 118, wherein the supernatant contains at least 100 mg L⁻¹ glucuronic acid.

122. The supernatant of claim 118, wherein the supernatant contains at least 500 mg L⁻¹ glucuronic acid.

123. The supernatant of claim 118, wherein the supernatant contains at least 100 mg L⁻¹ glucaric acid.

124. The supernatant of claim 118, wherein the supernatant contains at least 500 mg L⁻¹ glucaric acid.

125. A method of autonomously redirecting flux of glucose-6-phosphate in a recombinant cell, the method comprising regulating a phosphofructokinase-1 (pfk-1) in the recombinant cell, wherein the pfk-1 is regulated based on quorum sensing or nutrient sensing.

126. The method of claim 125, wherein regulating activity of the phosphofructokinase-1 protein comprises reducing the amount of phosphofructokinase-1 protein in the cell.

127. The method of claim 126, wherein the amount of phosphofructokinase-1 protein is reduced by at least 50%.

128. The method of claim 126, wherein the amount of phosphofructokinase-1 protein is reduced by at least 75%.

129. The method of claim 126, wherein the amount of phosphofructokinase-1 protein is reduced by at least 90%.

130. The method of any one of claims 126-129, wherein reducing the amount of phosphofructokinase-1 protein comprises degrading the phosphofructokinase-1 protein.

131. The method of any one of claims 126-130, wherein reducing the amount of phosphofructokinase-1 protein comprises targeting the phosphofructokinase-1 protein for degradation by a protease.

132. The method of any one of claims 125-131, wherein the phosphofructokinase-1 protein is fused to a peptide tag.

133. The method of claim 132, wherein the peptide tag is an SsrA tag.

134. The method of any one of claims 125-133, further comprising expressing in the cell an adaptor protein.

135. The method of claim 134, wherein the adaptor protein is SspB and targets the phosphofructokinase-1 protein for degradation.

136. The method of claim 135, wherein SspB is expressed under the control of a first inducible promoter.

137. The method of claim 136, wherein the first inducible promoter is responsive to a molecule produced by the recombinant cell.

138. The method of claim 136 or 137, wherein the first inducible promoter is responsive to a quorum sensing molecule.

139. The method of any one of claims 136-138, wherein the first inducible promoter is a P_{esaS} promoter or a P_{easR} promoter.

140. The method of any one of claims 136-139, wherein the first inducible promoter is from Pantoea stewartii.

141. The method of any of claims 138-140, wherein the quorum sensing molecule is 3-oxohexanoyl-homoserine-lactone (30C6HSL).

142. The method of any one of claims 125-141, further comprising expressing in the cell a gene encoding a quorum sensing transcription factor.

143. The method of claim 142, wherein the quorum sensing transcription factor is EsaR.

144. The method of claim 142 or 143, wherein the quorum sensing transcription factor is from Pantoea stewartii.

145. The method of any one of claims 125-144, further comprising expressing in the cell a gene encoding a quorum sensing molecule synthase.

146. The method of claim 145, wherein the quorum sensing molecule synthase is a 3OC6HSL synthase.

147. The method of claim 145 or 146, wherein the quorum sensing molecule synthase is EsaI.

148. The method of any one of claims 145-147, wherein the quorum sensing molecule synthase is from Pantoea stewartii.

149. The method of any one of claims 125-136, wherein the activity of pfk-1 is regulated based on the level of a nutrient.

150. The method of claim 149, wherein the nutrient is phosphate, arabanose, glucose or tryptophan.

151. The method of claim 149 or 150, wherein the first inducible promoter is responsive to a level of phosphate.

152. The method of any one of claims 149-151, wherein the first inducible promoter is a phoA promoter.

153. The method of any one of claims 149-152, wherein the first inducible promoter is a phoA promoter variant.

154. The method of any one of claims 149-153, wherein the phoA promoter variant is apFAB114 or apFAB104.

155. The method of any one of claims 149-154, further comprising contacting the cell with phosphate.

156. The method of claim 149 or 150, wherein the nutrient is arabanose.

157. The method of claim 156, wherein the first inducible promoter is responsive to a level of arabanose.

158. The method of claim 156 or 157, wherein the first inducible promoter is a P_{BAD} promoter.

159. The method of any one of claims 156-158, further comprising contacting the cell with glucose.

160. The method of any one of claims 156-159, further comprising contacting the cell with arabinose.

161. The method of any one of claims 156-160, further comprising contacting the cell with xylose.

162. The method of any one of claims 125-161, further comprising expressing in the cell a heterologous pathway that can utilize a glycolytic intermediate.

163. The method of any one of claims 125-162, wherein the glycolytic intermediate is glucose-6-phosphate.

164. The method of any one of claims 125-163, wherein the heterologous pathway comprises expressing a myo-inositol-1-phosphate synthase.

165. The method of any one of claims 125-164, further comprising reducing expression of a glucose-6-phosphate dehydrogenase (zwf).

166. The method of any one of claims 125-165, wherein the cell does not express glucose-6-phosphate dehydrogenase.

167. The method of any one of claims 164-166, wherein the myo-inositol-1-phosphate synthase (INO1) is expressed under the control of an inducible promoter.

168. The method of any one of claims 125-167, further comprising contacting the cell with a second inducer.

169. The method of claim 168, wherein the second inducer is isopropyl-β-D-1-thiogalactopyranoside.

170. The method of any one of claims 164-169, wherein the gene encoding the myo-inositol-1-phosphate synthase is a Saccharomyces gene.

171. The method of any one of claims 125-170, wherein the cell is a microbial cell.

172. The method of claim 171, wherein the microbial cell is a bacterial cell.

173. The method of claim 172, wherein the bacterial cell is an Escherichia coli cell.

174. The method of any one of claims 125-173, wherein the cell is a eukaryotic cell.

175. The method of claim 174, wherein the eukaryotic cell is a fungal cell, a yeast cell, an insect cell, a plant cell, or a mammalian cell.

176. The method of any one of claims 125-175, wherein the method is a method of producing myo-inositol, wherein the method further comprises culturing the cell and optionally recovering myo-inositol from the cell and/or cell culture.

177. The method of any one of claims 125-176, further comprising expressing in the cell a gene encoding a myo-inositol oxygenase.

178. The method of any one of claims 125-177, further comprising expressing in the cell a gene encoding a uronate dehydrogenase.

179. The method of any one of claims 125-178, wherein the method is a method of producing glucuronic acid, and the method further comprises culturing the cell and optionally recovering glucuronic acid from the cell and/or cell culture.

180. The method of any one of claims 125-179, wherein the method is a method of producing glucaric acid, and the method further comprises culturing the cell and optionally recovering glucaric acid from the cell and/or cell culture.

181. The method of any one of claims 125-180, wherein the method further comprises reducing expression of a glucarate dehydratase protein.

182. The method of any one of claims 125-181, wherein the method further comprises mutating a gudD gene in the recombinant cell.

183. The method of any one of claims 125-182, wherein the method further comprises reducing the expression of a uronate isomerase protein.

184. The method of any one of claims 125-183, wherein the method further comprises mutating a uxaC gene in the recombinant cell.

185. The method of any one of claims 125-184, further comprising reducing expression of the phosphofructokinase-II protein.

186. The method of claim 125-185, wherein reducing expression of the phosphofructokinase-II protein comprises eliminating expression the phosphofructokinase-II protein.

187. The method of any one of claims 125-186, wherein a gene encoding the adaptor protein is integrated into the genome of the cell.

188. The method of claim 187, wherein the gene encoding the adaptor protein is integrated at a phage attachment site.

189. The method of claim 188, wherein the phage attachment site is HK022.

190. A method for producing a recombinant cell, comprising expressing in the cell an autonomously regulatable phosphofructokinase protein (Pfk-1) and a means of autonomously regulating the Pfk-1 protein, wherein the Pfk-1 protein is regulated based on quorum sensing or nutrient sensing.

191. A recombinant cell, that expresses a regulatable phosphofructokinase protein (Pfk-1) and a means of autonomously regulating the Pfk-1 protein, wherein the Pfk-1 protein is regulated based on quorum sensing or nutrient sensing.
